SEQUENCE LISTING (17 sequences)

SEQ ID NO: 1 - RNA, 10 nt, synthetic construct
  source 1..10: mol_type = other RNA
tacagcatcg

SEQ ID NO: 2 - RNA, 11 nt, synthetic construct
  source 1..11: mol_type = other RNA
tacgacattcg

SEQ ID NO: 3 - RNA, 10 nt, synthetic construct
  source 1..10: mol_type = other RNA
tacagcatcg

SEQ ID NO: 4 - DNA, 10 bp, synthetic construct
  source 1..10: mol_type = other DNA
tacagcatcg

SEQ ID NO: 5 - RNA, 20 nt, synthetic construct
  source 1..20: mol_type = other RNA
tacagcatcggcctggacat

SEQ ID NO: 6 - RNA, 20 nt, synthetic construct
  source 1..20: mol_type = other RNA
tacagcatcgcgctggacat

SEQ ID NO: 7 - DNA, 20 bp, synthetic construct
  source 1..20: mol_type = other DNA
tacagcatcggcctggacat

SEQ ID NO: 8 - RNA, 30 nt, synthetic construct
  source 1..30: mol_type = other RNA
aatgatttaaataatttaaaagttcctact

SEQ ID NO: 9 - RNA, 50 nt, synthetic construct
  source 1..50: mol_type = other RNA
aatgatttaaataatttaaaagttcctactagaactggtaaattgtctac

SEQ ID NO: 10 - RNA, 50 nt, synthetic construct
  source 1..50: mol_type = other RNA
aatgatttaaataatttaaaagttcctactgaaactggtaaattgtctac

SEQ ID NO: 11 - RNA, 98 nt, synthetic construct
  source 1..98: mol_type = other RNA
gagaggttttcgcggtgcaagacgtttaacccgtcgaaaaaactcgtgtaaaacgtgttcgtgatttatttgaaaaatatgatatttcaactgatttt

SEQ ID NO: 12 - RNA, 20 nt, synthetic construct
  source 1..20: mol_type = other RNA
ggccggaccaaacgtttaat

SEQ ID NO: 13 - RNA, 19 nt, synthetic construct
  source 1..19: mol_type = other RNA
tacagcatcggcctggaca

SEQ ID NO: 14 - RNA, 21 nt, synthetic construct
  source 1..21: mol_type = other RNA
cagaaagagtgtctcatctta

SEQ ID NO: 15 - RNA, 21 nt, synthetic construct
  source 1..21: mol_type = other RNA
gactttcatcctggaaatata

SEQ ID NO: 16 - DNA, 21 bp, synthetic construct
  misc_feature 11: note = DNA
  source 1..21: mol_type = other DNA
  misc_feature 1..10: note = RNA
  misc_feature 12..21: note = RNA
ctagacctgttttgcttttgt

SEQ ID NO: 17 - RNA, 111 nt, synthetic construct
  source 1..111: mol_type = other RNA
gagaggttttcgcggtgcaagacgtttaacccgtcgtaaacatcgtgtaaaacgtgttcgtgatttatttgaaaaatatgatatttcaactgattttagaaatttaaatcc

(19) United States
(12) Patent Application Publication
(10) Pub. No.: US 2026/0237623 A1
(43) Pub. Date: Aug. 13, 2026

(54) SYSTEMS AND METHODS FOR DETERMINATION OF OLIGONUCLEOTIDE SEQUENCE BY TANDEM MASS SPECTROMETRY

(71) Applicant: Thermo Finnigan LLC, San Jose, CA (US)

(72) Inventors: Joshua Hinkle, San Jose, CA (US); James L. Stephenson, Raleigh, NC (US); John E.P. Syka, San Jose, CA (US); Joshua P. Salem, Ann Arbor, MI (US); William McGee, Beverly, MA (US); Robert L. Ross, Hopkinton, MA (US)

(21) Appl. No.: 19/409,135

(22) Filed: Dec. 4, 2025

Related U.S. Application Data
(63) Continuation-in-part of application No. PCT/US2024/032661, filed on Jun. 5, 2024.
(60) Provisional application No. 63/471,246, filed on Jun. 5, 2023.

(51) Int. Cl. H01J 49/00; G01N 33/68; H01J 49/42

(52) U.S. Cl. CPC H01J 49/0063; G01N 33/6848; H01J 49/005; H01J 49/4225

(57) ABSTRACT

Methods and systems for ion trap type resonant collision induced dissociation (CID) on labile compounds to drive selective dissociation along desired dissociation pathways are disclosed. Specifically, the present disclosure includes, systems, methods, and computer readable media (CRM) configured to introduce ions of at least one labile compound with multiple dissociation pathways into a trapping area of a dissociation cell. The dissociation cell is configured to generate a substantially quadrupolar radio frequency electrostatic confinement potential in the ion trapping region. The kinetic energy of selected precursor ions is increased while a selective dissociation parameter is maintained to cause the precursor ions to selectively dissociate primarily to first-generation products without being ejected from the trapping area. The selective dissociation parameter is determined in part by the Mathieu q value and controls the maximum kinetic energy or velocity of the ions.

### CROSS REFERENCE TO RELATED APPLICATION

This application is a continuation-in-part of International Patent Application No. PCT/US2024/032661, filed Jun. 5, 2024, which claims the benefit of and priority to the earlier filing date of U.S. Provisional Application No. 63/471,246, filed on Jun. 5, 2023, the entirety of each of these applications being incorporated herein by reference.

### SEQUENCE LISTING

The Sequence Listing is submitted as an XML file [“Sequence.xml, created on Jun. 5, 2024, 17,133 bytes] which is incorporated by reference herein.

### FIELD

The present disclosure is directed to systems and methods for determining oligonucleotide sequences using tandem mass spectrometry.

### BACKGROUND

The field of mass spectrometry offers a variety of tools and techniques for analyzing ions based upon their mass-to-charge (m/z) ratios. For example, a mass spectrometry system can include various devices for ionizing and isolating precursor ions for analysis, fragmenting the precursor ions into product ions, and/or mass analyzing the remaining precursor ions and/or newly generated productions. Current analytical methods that use mass spectrometry to fully sequence nucleic acids from either or both of the 5′ and 3′ ends are mainly limited to sequencing smaller oligonucleotides that are no longer than 20 bases in length. Thus, current mass spectrometry based methods cannot sequence larger oligonucleotides, such as guide RNA having approximately 100 nucleotides (32,320 Da), gene therapy oligonucleotides having roughly 1,800 nucleotides (577,059 Da), and oligonucleotides produced by primer QA/QC and solid phase synthesis having on the order of 100 nucleotides (30,449 Da). Additionally, modified oligonucleotides, such as oligomers comprising a phosphorothioate backbone, 2′ methoxy, 2′ fluoro, pseudouridine (base), methylated bases, 5′ double stranded duplexes, and siRNA have further limited the ability of mass spectrometry to sequence oligonucleotides.

Furthermore, current methods that do use mass spectrometry for oligonucleotide analysis produce a wide variety of fragment ion types. For example, higher energy beam type dissociation (HCD) produces a high degree of fragmentation (mainly low charge state product ions), which can only usefully provide sequence information for up to 20 bases. For oligonucleotides larger than 20 bases, data produced using HCD is difficult to interpret and does not cover complementary 5′ to 3′ sequence coverage.

Therefore, there is significant demand for the development of improved methodologies for both confirmational and de novo sequencing approaches applied to oligonucleotides. The goals of any MS based methodology should be centered around the production of complementary fragmentation and reduction of the multistep fragmentation process resulting in simplification of the data, for any given charge state.

### BRIEF DESCRIPTION OF THE DRAWINGS

FIG. 1 illustrates an example environment that includes an example mass spectrometer system for thorough mass spectrometry experiments and measurements using ion trap type resonance induced collision induced dissociation (CID) at low q values on labile compounds to drive selective dissociation along desired dissociation pathways.

FIG. 2 is schematic showing a representative aspect of a mass spectrometry system configured to perform collision induced dissociation (CID).

FIG. 3A is a schematic illustrating one aspect of the disclosed method comprising negative ion electron transfer dissociation (NETD), wherein an electron transfer reagent is reacted with an oligomer to generate one or more fragments of the oligomer or produce electron transfer with no prompt fragmentation (ETnoD).

FIG. 3B is a schematic illustrating an aspect of the disclosed method comprising photodetachment of a precursor species at 213 nm wavelength and the use of infrared multiphoton dissociation (IRMPD for producing sequence specific product ions from oligonucleotide anions.

FIG. 4 is a schematic diagram illustrating a representative aspect of a fragmentation device and further illustrates associated control components.

FIG. 5 is a graph of normalized characteristic frequency (β) as a function of trapping parameter (q) showing that the relationship between the quantities β and q can at least partially characterize a stability with which an ion can be trapped within a fragmentation device.

FIG. 6 is a schematic comparing mRNA contained within a lipid-based vaccine delivery system (e.g., nanoparticle), a basic oligonucleotide, and a modified oligonucleotide demonstrating the compositional challenges for sequencing that arise from the differences in basic oligonucleotide and modified oligonucleotide structures.

FIG. 7 is the chemical structure of a negatively charged oligonucleotide, wherein the charge resides on the phosphodiester backbone and the nucleotides are purine or pyrimidine bases.

FIG. 8 provides the sequences for commercial RNA therapeutics Givosiran (SEQ ID NO: 14), Lumasiran (SEQ ID NO: 15), and Inclisiran (SEQ ID NO: 16), which are approximately 20mers and heavily modified.

FIG. 9 is a schematic depicting the complexity of an exemplary lipid nanoparticle illustrating the nanoparticle considerations such as size heterogeneity, lipid composition, and mRNA vaccine payload.

FIG. 10 is a schematic depicting the nomenclature of oligonucleotide cleavage points a, b, c, and d, for fragments produced from the 5′ terminus; and w, x, y, and z for fragments from the 3′ terminus.

FIG. 11 is a schematic demonstrating internal fragmentation and base loss fragmentation.

FIG. 12 is a schematic of an aspect of a disclosed method comprising at least a partial mRNA enzymatic digest, such as by using immobilized ribonucleases on beads or in a column wherein the mRNA has a 5′cap structure, a 5′UTR with Kozak translation initiation sequence, main sequence/open reading frame (ORF), 3′ UTR with globin sequence, and a 3′ poly(A) tail; where digest fragments are analyzed by LC MS/MS.

FIG. 13 is a schematic of an aspect of the method disclosed herein comprising using Electrospray Ionization (ESI) to form a negatively charged micelle containing RNA, removal of a lipid or micelle, followed by ion activation of the RNA resulting in fragmented mRNA ions sequenced with low q CID.

FIG. 14 is a schematic illustrating an aspect of the disclosed method herein comprising low q CID for the direct sequencing of an mRNA 30mer having SEQ ID NO: 8 in a N-Dodecyl-β-D-Maltoside (DDM) micelle.

FIG. 15 is a mass spectrum produced using an aspect of the disclosed method herein comprising low q CID that provides complementary 5′ and 3′ sequence information for an RNA 50mer having SEQ ID NO: 10.

FIG. 16 compares mass spectra data produced using CID (top) compared to lower energy HCD (middle), and higher energy HCD (lower) that provides complementary 5′ and 3′ sequence information for an RNA 50mer having SEQ ID NO: 10 from the −10 charge state.

FIG. 17 compares mass spectra data from 1620 m/z to 1820 m/z that was produced using CID (top) compared to lower energy HCD (middle), and higher energy HCD (lower), of an RNA 50mer having SEQ ID NO: 10 from the −10 charge state.

FIG. 18 compares mass spectra data from 1811 m/z to 1836 m/z for spectrum produced using CID (top) versus HCD at lower energy (middle) and higher energy (bottom) of a fragmented precursor ion comprising a 50mer having SEQ ID NO: 10, wherein the example comprising CID (top) demonstrated efficient, reproducible production of a y₄₀ (−7) ion and a y₄₆ (−8) ion.

FIG. 19 provides static spray (top) mass spectra data for an oligomer sequence (SEQ ID NO: 10) AAU GAU UUA AAU AAU UUA AAA GUU CCU ACU GAA ACU GGU AAA UUG UCU AC compared to LC data (bottom) for distinguishing a single base change of the sequence, wherein the sequence (SEQ ID NO: 9) is AAU GAU UUA AAU AAU UUA AAA GUU CCU ACU AGA ACU GGU AAA UUG UCU AC (swapping GA to AGA).

FIG. 20 provides mass spectrometry data identifying c-ions produced from an oligomer sequence (SEQ ID NO: 10) AAU GAU UUA AAU AAU UUA AAA GUU CCU ACU GAA ACU

GGU AAA UUG UCU AC compared to the single base swap of the oligomer having a sequence (SEQ ID NO: 9) AAU GAU UUA AAU AAU UUA AAA GUU CCU ACU AGA ACU GGU AAA UUG UCU AC (changing GAA to AGA) illustrating c31 (−7) for adenine (GAA) having a mass/charge ratio equal to 1411.58, and guanine (AGA) having a mass/charge ratio of 1413.74 thereby establishing that disclosed embodiments can distinguish and accurately determine the sequence of 50mers (two base swap) by comparing (c-ions).

FIG. 21 provides mass spectrometry data including identifying y-ions produced from an oligomer sequence (SEQ ID NO: 10) AAU GAU UUA AAU AAU UUA AAA GUU CCU ACU GAA ACU GGU AAA UUG UCU AC compared to a single base change for the oligomer having sequence (SEQ ID NO: 9) AAU GAU UUA AAU AAU UUA AAA GUU CCU ACU AGA ACU GGU AAA UUG UCU AC (changing GAA to AGA) illustrating y19 (−3) for adenine (GAA) having a mass/charge ratio equal to 2008.60 and illustrating y19 (−3) for guanine (AGA) having a mass/charge ratio equal to 2014.91 thereby establishing that disclosed embodiments can distinguish and accurately determine the sequence of 50mers having a single base change by comparing (y-ions).

FIG. 22 is the frequency data (relative absorbance versus time) (top) of a 98mer at 4.02 minutes, a 200mer at 5.48 minutes, a 300mer at 5.96 minutes, 750mer at 6.24 minutes, and a 1000mer at 6.59 minutes and the complete full scan mass spectrum (bottom) of a 98mer having SEQ ID NO: 11.

FIG. 23 is a chromatograph of a 15 minute chromatographic run of a mRNA 98mer molecule having a sequence G-A-G-A-G-G-U-U-U-U-C-G-C-G-G-U-G-C-A-A-G-A-C-G-U-U-U-A-A-C-C-C-G-U-C-G-A-A-A-A-A-A-C-U-C-G-U-G-U-A-A-A-A-C-G-U-G-U-U-C-G-U-G-A-U-U-U-A-U-U-U-G-A-A-A-A-A-U-A-U-G-A-U-A-U-U-U-C-A-A-C-U-G-A-U-U-U-U (SEQ ID NO: 11), which was designed to include a number of repeat sequences to increase sequencing difficulty.

FIG. 24 is a mass spectrum produced using an aspect of the disclosed method herein comprising low q CID showing the mass to charge ratio for y ions and c ions that provides complementary 5′ and 3′ sequence information for an mRNA 98mer having SEQ ID NO: 11.

FIG. 25 is the product ion mass spectrum of FIG. 24 showing a mass/charge ratio from 970 to 1320 from a −23 charge state produced using an aspect of the disclosed method using low q CID for sequencing an mRNA 98mer molecule having SEQ ID NO: 11.

FIG. 26 is a mass spectrum produced using an aspect of the disclosed method herein comprising low q CID showing the mass to charge ratio for y ions and c ions that provides complementary 5′ and 3′ sequence information for an mRNA 98mer having SEQ ID NO: 11.

FIG. 27 is a mass spectrum produced using an aspect of the disclosed method herein comprising low q CID showing the mass to charge ratio for y ions and c ions that provides complementary 5′ and 3′ sequence information for an mRNA 111mer having SEQ ID NO: 17.

FIG. 28 provides a mass spectrum for the negative ion electron transfer dissociation (NETD) of an mRNA 10mer having SEQ ID NO: 2 reacted with fluoranthene with a starting charge state having a mass/charge ratio of 784.85 and limited prompt fragmentation at mass/charge ratio of 971.45, at a mass/charge ratio 111.3.83, and at a mass/charge ratio of 1457.17. The major peaks in the spectrum represent consecutive electron transfer events with no prompt fragmentation or ETnoD.

FIG. 29 provides the mass spectrum of an mRNA 10mer having SEQ ID NO: 2 demonstrating that primarily d- and w-type ions were produced for ETnoD followed by low q CID.

FIG. 30 provides ESI mass spectrometry data for an mRNA 20mer having sequence 5′-U ACAGCAUCGGCCUGGACAU-3 (SEQ ID NO: 5).

FIG. 31 demonstrates the photodetachment process of mRNA 20mer having SEQ ID NO: 5 using a high pressure trap and a wavelength of 213 nm at 8 ms irradiation time with a start point having a mass/charge ratio of 761.59; −1e having a mass/charge ratio of 870.39; −2e at 1015.45; −3e at 1218.54; −4e at 1523.17; illustrating very minimal prompt fragmentation.

FIG. 32 demonstrates the photodetachment process and related prompt fragmentation of an mRNA 20mer having SEQ ID NO: 5 comprising a phosphorothioate backbone in a high pressure trap having a start point with a mass/charge ratio of 832.51; −1e at mass/charge ratio of 951.73; −2e at a mass/charge ratio of 1110.35; −3e at 1332.41; and a −4e at a mass/charge ratio of 1665.52; wherein it was demonstrated that the phosphorothioate backbone absorbs much more readily at 213 nm producing higher levels of prompt fragmentation.

FIG. 33 is a 10× magnified mass spectrometry data from 1120 m/z to 1320 m/z for an mRNA 20mer having SEQ ID NO: 5 oligonucleotide comprising a phosphorothioate backbone produced via photodetachment/prompt fragmentation in a high-pressure trap.

FIG. 34 provides a full scan mass spectrum of an mRNA 20mer having SEQ ID NO: 5.

FIG. 35 shows a product ion mass spectrum produced by photodetachment of the precursor ion having SEQ ID NO: 5 with a m/z ratio of 1059 (see FIG. 34) followed by low q CID of the odd electron m/z 1271 ion.

FIG. 36 is the mass spectrum produced by photodetachment at m/z 951 (−7) followed by low q CID of the odd electron ion at a m/z 1110 (−6) for a 20mer with a phosphorothioate backbone having SEQ ID NO: 5.

FIG. 37 is an ESI mass spectrum for RNA 20mer having SEQ ID NO: 5 using a linear ion trap as an ion detection device.

FIG. 38 is a product ion mass spectrum for an mRNA 20mer having SEQ ID NO: 5 produced using the default scan mode of the linear ion trap to detect the product ions generated from a low q CID experiment.

FIG. 39 is a product ion mass spectrum for an mRNA 20 having SEQ ID NO: 5 produced using an enhanced scan mode (slower scan rate) in order to increase the resolution of the linear ion trap to detect or characterize the isotopic envelopes of higher charge states at baseline resolution.

FIG. 40 is an ESI mass spectrum produced using ESI MS of double stranded RNA having SEQ ID NO: 12.

FIG. 41 is a product ion mass spectrum comprising MS/MS at −8 charge state of dsRNA having SEQ ID NO: 12, wherein MS2 provides for ds strand separation and MS3 yields strand sequencing information.

FIG. 42 provides mass spectra for MS/MS at −8 charge state of dsRNA having SEQ ID NO: 12 directly for CID (top) versus HCD at 10% RCE (bottom).

FIG. 43 is a product ion mass spectrum produced from the ESI of DDM micelles surrounding a 30mer having SEQ ID NO: 8 illustrating the DDM peaks having a m/z ratio from 539.27 to 1019.60 and the mRNA 30mer having SEQ ID NO: 8 at a −5 charge state at m/z 1936.06.

FIG. 44 is a product ion mass spectrum (×7) for the low q CID data o −5 charge state of an RNA 30mer having SEQ ID NO: 8 that was previously embedded in a DDM micelle.

FIG. 45 is a product ion mass spectrum of an RNA 20mer having SEQ ID NO: 6 at a q of 0.25 (top) and 0.15 respectively (bottom).

FIG. 46 is an expanded view of FIG. 45 for a q of 0.25 (top) and 0.15 (bottom) showing the difference in data quality produced as a result of the lower ion velocities at low q.

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand. In the accompanying sequence listing:

(Reference)
SEQ ID NO: 1
5′ U-A-C-A-G-C-A-U-C-G 3′.
(Scramble)
SEQ ID NO: 2
5′ U-A-C-G-A-C-A-U-C-G 3′.
(2′-O-methyl)
SEQ ID NO: 3
5′ U-A-C-A-G-C-A-U-C-G 3′.
(DNA analog)
SEQ ID NO: 4
5′ T-A-C-A-G-C-A-T-C-G 3′.
(Full PS 20 mer)
SEQ ID NO: 5
5′ U-A-C-A-G-C-A-U-C-G-G-C-C-U-G-G-A-C-A-U 3′.
(Minus 1 PS 20 mer)
SEQ ID NO: 5
5′ U-A-C-A-G-C-A-U-C-G-G-C-C-U-G-G-A-C-A-U 3′.
(Minus 2 PS 20 mer)
SEQ ID NO: 5
5′ U-A-C-A-G-C-A-U-C-G-G-C-C-U-G-G-A-C-A-U 3′.
(Minus 3 PS 20 mer)
SEQ ID NO: 5
5′ U-A-C-A-G-C-A-U-C-G-G-C-C-U-G-G-A-C-A-U 3′.
(Minus 4 PS 20 mer)
SEQ ID NO: 5
5′ U-A-C-A-G-C-A-U-C-G-G-C-C-U-G-G-A-C-A-U 3′.
(Minus 5 PS 20 mer)
SEQ ID NO: 5
5′ U-A-C-A-G-C-A-U-C-G-G-C-C-U-G-G-A-C-A-U 3′.
(Minus 6 PS 20 mer)
SEQ ID NO: 5
5′ U-A-C-A-G-C-A-U-C-G-G-C-C-U-G G-A-C-A-U 3′.
(Minus 7 PS 20 mer)
SEQ ID NO: 5
5′ U-A-C-A-G-C-A-U-C-G-G-C-C-U-G-G-A-C-A-U 3′.
(Minus 8 PS 20 mer)
SEQ ID NO: 5
5′ U-A-C-A-G-C-A-U-C-G-G-C-C-U-G-G-A-C-A-U 3′.
(Minus 9 PS 20 mer)
SEQ ID NO: 5
5′ U-A-C-A-G-C-A-U-C-G-G-C-C-U-G-G-A-C-A-U 3′.
(Minus 10 PS 20 mer)
SEQ ID NO: 5
5′ U-A-C-A-G-C-A-U-C-G-G-C-C-U-G-G-A-C-A-U 3′.
(Reference)
SEQ ID NO: 5
5′ U-A-C-A-G-C-A-U-C-G-G-C-C-U-G-G-A-C-A-U 3′.
(Scramble)
SEQ ID NO: 6
5′ U-A-C-A-G-C-A-U-C-G-C-G-C-U-G-G-A-C-A-U 3′.
(DNA analog)
SEQ ID NO: 7
5′ T-A-C-A-G-C-A-T-C-G-G-C-C-T-G-G-A-C-A-T 3′.
(RNA 30 mer)
SEQ ID NO: 8
5′ A-A-U-G-A-U-U-U-A-A-A-U-A-A-U-U-U-A-A-A-A-
G-U-U-C-C-U-A-C-U-3.
(RNA 50 mer single base swap)
SEQ ID NO: 9
5′ A-A-U-G-A-U-U-U-A-A-A-U-A-A-U-U-U-A-A-A-A-
G-U-U-C-C-U-A-C-U-A-G-A-A-C-U-G-G-U-A-A-A-U-
U-G-U-C-U-A-C 3′.
(RNA 50 mer single base swap)
SEQ ID NO: 10
5′ A-A-U-G-A-U-U-U-A-A-A-U-A-A-U-U-U-A-A-A-A-
G-U-U-C-C-U-A-C-U-G-A-A-A-C-U-G-G-U-A-A-A-U-
U-G-U-C-U-A-C 3′.
(RNA 98 mer)
SEQ ID NO: 11
5′ G-A-G-A-G-G-U-U-U-U-C-G-C-G-G-U-G-C-A-A-G-
A-C-G-U-U-U-A-A-C-C-C-G-U-C-G-A-A-A-A-A-A-C-
U-C-G-U-G-U-A-A-A-A-C-G-U-G-U-U-C-G-U-G-A-U-
U-U-A-U-U-U-G-A-A-A-A-A-U-A-U-G-A-U-A-U-U-U-
C-A-A-C-U-G-A-U-U-U-U 3′.
(DS RNA)
SEQ ID NO: 12
5′ G-G-C-C-G-G-A-C-C-A-A-A-C-G-U-U-U-A-A-U 3′.
(RNA 19 mer)
SEQ ID NO: 13
5′ U-A-C-A-G-C-A-U-C-G-G-C-C-U-G-G-A-C-A 3′.
(RNA 21 mer)
SEQ ID NO: 14
5′ C-A-G-A-A-A-G-A-G-U-G-U-C-U-C-A-U-C-U-U-A 3′.
(RNA 21 mer)
SEQ ID NO: 15
5′ G-A-C-U-U-U-C-A-U-C-C-U-G-G-A-A-A-U-A-U-A 3′.
(RNA 21 mer)
SEQ ID NO: 16
5′ C-U-A-G-A-C-C-U-G-U-T-U-U-G-C-U-U-U-U-G-U 3′.
(RNA 111 mer)
SEQ ID NO: 17
5′ G-A-G-A-G-G-U-U-U-U-C-G-C-G-G-U-G-C-A-A-G-
A-C-G-U-U-U-A-A-C-C-C-G-U-C-G-U-A-A-A-C-AU-C-
G-U-G-U-A-A-A-A-C-G-U-G-U-U-C-G-U-G-A-U-U-U-
A-U-U-U-G-A-A-A-AA-U-A-U-G-A-U-A-U-U-U-C-A-A-
C-U-G-A-U-U-U-U-A-G-A-A-A-U-U-U-A-A-A-U-C-C 3′.

### DETAILED DESCRIPTION

### I. Terms, Ranges, and Definitions

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

The method embodiments described herein should not be construed as limiting in any way. Instead, the present disclosure is directed toward all novel and non-obvious features and aspects of the present disclosure, alone and in various combinations and sub-combinations with one another. The disclosed methods are not limited to any specific aspect or feature or combinations thereof, nor do the disclosed methods require that any one or more specific advantages be present or problems be solved. Any theories of operation are to facilitate explanation, but the methods are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed device and method can be used in conjunction with other devices and methods. Additionally, the description may use terms like “produce” and “provide” to describe the disclosed methods. These terms are high-level abstractions of the actual operations that are performed. The actual operations that correspond to these terms will vary depending on the particular implementation and will be understood by a person of ordinary skill in the art. Furthermore, examples may be described with reference to directions indicated as “above,” “below,” “upper,” “lower,” and the like. These terms are used for convenient description, but do not imply any particular spatial orientation unless so indicated.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood by a person of ordinary skill in the art to which this disclosure belongs.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

### II. Brief Overview

Methods and systems for ion trap type resonant collision induced dissociation (CID) on labile compounds to drive selective dissociation along desired dissociation pathways are disclosed. Specifically, the present disclosure teaches systems, methods, and computer readable media (CRM) configured to inject, create or otherwise introduce ions of at least one ionic labile compound with multiple dissociation pathways into a trapping area of a dissociation cell configured to generate a substantially quadrupolar radio frequency electrostatic confinement potential in the ion trapping region, and then increasing the kinetic energy of the ions to a selective dissociation parameter to cause the labile compound ions selectively dissociate without being ejected from the trapping area.

Embodiments of the present disclosure perform ion trap type resonant collisional activation (CID) using a reduced precursor ion activation Mathieu q value (approximately proportional to ion resonant frequency) reduces the upper limit of the kinetic energy (KE) of activating collisions not available through adjustment of the auxiliary field amplitude (NCE) and suppresses the yield of unwanted internal and base loss fragment ion species. In conventional ion trap type resonant CID, non-labile species are kinetically excited by the resonant auxiliary AC field, which is converted to internal vibrational energy via collisions, and tend to undergo a single bond cleavage to produce two product ions. The product ions are typically no longer resonant with the applied auxiliary AC field and will rapidly cool before further dissociation or bond cleavage occurs. However, certain labile species such as deprotonated oligonucleotides (i.e., anions), RNA, negative peptide, or negative protein ion species operate differently and do not immediately undergo single-bond cleavage upon application of resonant energy. Rather, these labile species accumulate additional internal energy through further collisions (e.g., storing as vibrational energy) beyond that which is needed to cause a single-bond cleavage. When these “overheated” labile species finally begin to dissociate, they often undergo a cascade of simultaneous or sequential fragmentation processes at multiple locations in the molecule (e.g., producing internal or base loss fragment ion species). The resulting fragments create a far more complicated spectrum and are more difficult to unambiguously assign to particular product ions, reducing sensitivity and specificity of precursor identification and quantitation. The problem is particularly amplified in the case of oligonucleotides where the composition of different molecules is drawn from a limited set of amino acids, creating many possible interferences when these molecules are multiply fragmented.

Systems, methods, and computer-readable media taught herein can overcome these issues by gently dissociating labile species to predominantly produce a single generation of product ions, for example, including a single bond cleavage. The resulting product ion spectra are dominantly composed of first-generation products (sequence ions and base losses from the precursor). The reduction in product ion spectral complexity in favor of sequence informative species simplifies and increases the confidence of m/z peak annotation and therefore confirmation or elucidation of the primary RNA sequence. The low q CID methodology (lqCID) described here builds on previous studies involving optimization of the auxiliary field amplitude¹ to limit activation energy and effect such spectral simplification. The lqCID approach is applicable to both even electron RNA precursor ions obtained from electrospray ionization or odd electron intact anionic species such as those derived from non-dissociative negative ion electron transfer reactions (so called NETnoD) reactions or activated electron photo-detachment of said electrospray generated precursor ions.

For example, a key application of the methods, systems, and computer readable media (CRM) of the present disclosure is for the analysis and sequencing of oligonucleotides using a mass spectrometer capable of generating sequence specific information on all nucleic acid types via one or more stages of tandem mass spectrometry. In various embodiments, the present disclosure allows for sequencing of oligonucleotide ions, or generated anions, having at least 20mer, 30mer, 50mer, 100mer, 200mer, 500mer, 1000mer, 1800mer, or greater.

As used herein, a “labile” ionic compound or species is an oligonucleotide (particularly anions and, more particularly, deprotonated oligonucleotide anions), a peptide anion, a protein anion, or complexes of these components. These labile compounds have multiple dissociation pathways that produce different types of bond cleavages and different types of product ions when subjected to ion trap type resonant CID. In other words, the labile compound has multiple bonds that are equally susceptible to cleavage when overheated or have a tendency to predominantly undergo multiple fragmentation under conventional ion trap type resonant CID conditions. Examples of labile species include RNA, modified RNA, DNA, Single stranded DNA, synthetic RNA analogs, or a combination thereof. RNA can include mRNA, tRNA rRNA, siRNA, circular RNA, double stranded RNA, RNA-DNA complexes, RNA aptamers, RNA modified with carbohydrates, peptides, proteins, and RNA-protein complexes. The labile species can include electron deficient deprotonated oligonucleotide, peptide, or protein anions such as those produced by photo-electron detachment or electron transfer (abstraction) ion-ion reactions of deprotonated oligonucleotide anions with suitable negative electron transfer reagent cations.

Also disclosed herein is a mass spectrometer, comprising an ion source for generating precursor ions comprising oligonucleotides ions; a dissociation cell positioned downstream of the ion source and configured to receive the precursor ions and to trap the precursor ions therein for a trapping period as disclosed herein, wherein the dissociation cell is configured to collide the precursor ions with a buffer gas to fragment the precursor ions into fragment ions; and a mass analyzer positioned downstream of the dissociation cell and configured to mass analyze the fragment ions. As used herein, “downstream” indicates that the ions can leave a first area to travel from the first area to a second area that is downstream of the first area. In other words, the ions can travel from the dissociation cell to the mass analyzer. In other aspects, the dissociation cell is a linear ion trap. In other aspects the dissociation cell is a multipole field-based ion trap such as a quadrupole-based ion trap that operates by forming a quadrupole field. The mass spectrometer may further comprise an RF (Radio Frequency) power supply configured to apply a trapping voltage waveform to a first subset of the plurality of electrodes to generate an RF quadrupole electric field; and an alternating current (AC) supply to apply an auxiliary AC voltage waveform to a second subset of the plurality of electrodes to generate an applied auxiliary electric field. The frequency of the AC supply can be less than the frequency range that would traditionally be considered “radio frequency” in some examples. The first subset of the plurality of electrodes may comprise a first pair of opposed electrodes spaced apart along a first axis and a second pair of opposed electrodes spaced apart along a second axis that is at least substantially perpendicular to the first axis. The dissociation cell can be configured to generate the RF quadrupole electric field by applying the trapping voltage waveform to each of the first pair of opposed electrodes with a first phase; and applying the trapping voltage waveform to each of the second pair of opposed electrodes with a second phase that is 180 degrees out of phase with the first phase. The dissociation cell can be configured to generate the applied auxiliary electric field by applying the auxiliary AC voltage waveform to the second subset of the plurality of electrodes. The applied auxiliary voltage may be applied between said first pair of opposed electrodes, between said second pair of opposed electrodes or between opposed electrodes of both said first pair and said second pair of opposed electrodes. The mass spectrometer may further comprise an RF power supply configured to apply the trapping voltage waveform and an AC power supply configured to apply the auxiliary voltage waveform to the electrodes of the dissociation cell.

The foregoing and other objects, features, and advantages of the invention will become more apparent from the following detailed description, which proceeds with reference to the accompanying figures.

While non-limiting, the following simple representative calculations can be used to illustrate the performance of some embodiments of the present invention. In the exemplary discussion and equations disclosed below the equations of motion are in MKS units. However, a person having skill in the art would understand that these units may be converted to Daltons, AMU, etc. Because of this, some of the representations below include a conversion factor that allow for conversion between with ion mass and mass-to-charge ratios in practical units. Specifically, we introduce the scale factor η and the ion mass in Daltons, mᵢₒₙ such that we have:

$${𝓂}_{ion} = η {m}_{ion}$$

$$where :$$

$$η ≅ 1.660539066 \times {10}^{- 27} kg / Da$$

and the quantity mᵢₒₙ/zᵢₒₙ has units of Da/elementary charge or equivalently, Thompson, which is an often used proposed unit for ion mass-to-charge ratio in the field of mass spectrometry.

In dissociation cells according to the present disclosure, an ion of mass, mᵢₒₙ and charge zᵢₒₙe, where e is the elementary charge and zᵢₒₙ is the number of elementary charges of the ion (the ion's charge state) in a generic radio frequency (RF) trapping (or confinement) potential, Φ(\vec{R}, t) that satisfies the LaPlace condition (no/negligible space charge potential) where \vec{R} then is the ion's displacement in the time variant potential, the trapping potential can be represented as:

$$\vec{E} ( \vec{R} , t ) = - ∇ Φ ( \vec{R} , t )$$

For simplicity we will assume this trapping potential is the product of an exclusively time variant function, V(t), and an exclusively spatially dependent function g(\vec{R}). V(t) is the scaling magnitude of the RF voltages applied to the electrodes that establish the potential and g(\vec{R}) is a normalized potential that is scaled by these voltages. The assumption is the ratio of the magnitudes of the voltages applied to the various trapping-potential-establishing electrodes are fixed just as the shapes of the electrodes are fixed. So g(\vec{R}) defines the “spatial shape” of the potential and V(t) defines how its overall magnitude varies with time. In practice this is a pretty good model for how the RF trapping (confinement) potentials in practical RF ion trapping devices and RF quadrupole ion trap devices of the sort that those of ordinary skill in the art build and incorporate into modern mass spectrometer instruments. So, the trapping potential is expressed as:

$$Φ ( \vec{R} , t ) = V ( t ) g ( \vec{R} )$$

and its associated electric field is:

$$\vec{E} ( \vec{R} , t ) = - ∇ Φ ( \vec{R} , t ) = V ( t ) \vec{G} ( \vec{R} )$$

$$Where :$$

$$\vec{G} ( \vec{R} ) = - ∇ g ( \vec{R} ) .$$

A core application of the present disclosure involves m/z selective kinetic activation of oligonucleotide ions. This means the confining RF potential must be a “substantially quadrupolar” or perhaps more accurately “dominantly quadrupolar” at least in the dimension(s) and spatial region where the ions are being kinetically activated.
An arbitrary 2D electrostatic potential in a region without space charge, Φ_{arb}, can be represented in polar coordinates as

$${Φ}_{arb} ( r , θ ) = {{\sum }_{n = 0}}^{∞} {a}_{n} ( {r}^{n} Cos n θ ) + {{\sum }_{n = 1}}^{∞} {b}_{n} ( {r}^{n} Sin n θ )$$

Where the aₙ and bₙ scaling coefficients weight the individual multipole potentials. The sine and cosine angular terms just represent rotations of any particular order, n, multipole potential. The zeroth order (n=0) order multipole potential is simply 1−a spatially uniform potential which therefore is gradient free. The reason they are called multipole potentials is that for a fixed radial displacement, r, the potential will go through 2n total maxima and minima (poles) as θ is varied from 0 to 2π. The first order multipoles (n=1) represent rotations of parallel plate capacitor potentials, one normal to the x-axis and one normal to the y-axis, and are referred to as dipole potentials. These potentials vary linearly with x and y displacements respectively and their respective gradients are uniform. The second order multipole potentials (n=2) are referred to as quadrupole potentials as 2n=4. The quadrupole potential varies quadratically with radial displacement, and, with a proper choice of coordinates, the potential gradient in each cartesian dimension is proportional to displacement in that dimension alone. This is not true for the higher order multipoles.

In some embodiments, the multipole potentials correspond to RF 2D quadrupole potentials that are pure (ideal quadrupole potential) or have small amounts of higher order multipole components and superposed auxiliary alternating current (AC) dipole potentials. For embodiments where the dissociation cell is an RF 2D (linear) quadrupole ion trap, a person having skill in the art would understand that we can choose our coordinates so that only the rⁿ cos nθ terms are relevant to the discussion.

According to the present disclosure, Newton's second law can be used to show that the motion of ions in an RF trapping potential as described above Φ(\vec{R}, t) can be represented as:

$${𝓂}_{ion} = \frac{{d}^{2} \vec{R}}{{dt}^{2}} = \vec{F} ( \vec{R} , t )$$

where \vec{F}(\vec{R}, t) is the force on the ion in the RF trapping potential field,

$$\frac{{d}^{2} \vec{R}}{{dt}^{2}} ,$$

is the ion's acceleration and the mᵢₒₙ is the mass of the ion. According to the present disclosure, the force of the ion is simply the product of the electric field associated with the trapping potential and the charge of the ion and is given as:

$$\vec{F} ( \vec{R} , t ) = ( {𝓏}_{ion} e ) \vec{E} ( \vec{R} , t )$$

where e is the elementary charge and zᵢₒₙ is the number of elementary charges on the ion. In some examples of the present disclosure, the confining RF potential will be sinusoidally varying with time such that

$$g ( t ) = {V}_{RF} Cos ( {ω}_{RF} t ) .$$

Substituting this into our expression for the electric field we get:

$$\vec{F} ( \vec{R} , t ) = ( {𝓏}_{ion} e ) {V}_{RF} Cos ( {ω}_{RF} t ) \vec{G} ( R ) .$$

This in turn gives us the general equation of motion for the ion in our generic sinusoidally time variant trapping potential as:

$${𝓂}_{ion} \frac{{d}^{2} \vec{R}}{{dt}^{2}} = ( {𝓏}_{ion} e ) {V}_{RF} Cos ( {ω}_{RF} t ) \vec{G} ( R )$$

This gives us a general equation of motion upon substitution and rearrangement as follows:

$$\frac{{d}^{2} \vec{R}}{{dt}^{2}} - \frac{e / η}{( {𝓂}_{ion} / {𝓏}_{ion} )} {V}_{RF} Cos ( {ω}_{RF} t ) \vec{G} ( \vec{R} ) = 0$$

which gives a general equation of motion for the ions of any type of RF potential including the RF potential within RF 2D (linear) quadrupole ion traps in the absence of any superposed auxiliary AC potentials.

A person having skill in the art, particularly with the benefit of the present disclosure, would understand that the preceding representations have ignored ion collisions with neutrals. For example, the ion trapping region of a dissociation cell may include a buffer gas (also referred to as a damping gas or even a collision gas) at a particular pressure such as approx. 6×10⁻³ Torr (6 mTorr) Helium, and/or approximately 1-to-2×10⁻⁵ Torr of Nitrogen. Such a buffer gas is used in concert with a superposed auxiliary potential on the RF trapping potential to resonantly kinetically excite the ions and, through collisions with neutrals, convert some of that kinetic energy into internal (vibrational) energy which cause the ions to dissociate. Further it is well understood in the art that collisions with the buffer gas molecules may also be used to kinetically cool ions that are injected into the dissociation cell to effect their trapping in the device. Collisions with a buffer gas can also cool newly created fragments that are no longer resonantly excited by the field that produced the fragmentation of the parent ion. Buffer gases suitable for use with examples of systems and methods taught herein include, without limitation, helium, diatomic hydrogen, diatomic deuterium, diatomic nitrogen and argon.

We can then represent the ion trajectory within the example RF 2D (linear) quadrupole ion trap by using the more general approach of Gerlich which involves treating the action of the ion as if is made of two components. This allows an ion's trajectory, \vec{R}(t) to be treated as having two components such that:

$$\vec{R} ( t ) = \vec{S} ( t ) + \vec{W} ( τ )$$

where \vec{S}(t) represents a slow component of motion of the ion such that \vec{S}(t) only changes a relatively small amount through a cycle of the trapping potential field. In the literature it is variously referred to as drift, bulk, secular motion, etc. [symbol] (t) represents the motion of the ion in direct response to the sinusoidal cycling of the RF potential. It is herein assumed in this example embodiment that the mass-to-charge ratio of the ion is such that the ion just undergoes a sinusoidal wiggle in response to each cycle of the trapping potential such that the ion returns almost to the same position. That net change in position that occurs in a single period of the trapping potential is “small,” and such change in position is accounted for by \vec{S}(t). This micromotion shall be discussed as wiggle motion within the present disclosure. Where the ion is sufficiently massive relative to the strength of the electrical forces such that the amplitude of the wiggle is “small” (e.g., such that the electric field doesn't change significantly throughout the range of the ion's secular motion), the electric field can be treated as being locally uniform. This allows us to estimate the equation of motion for the wiggle component of the ion's motion over an RF cycle as:

$$η {m}_{i o n} \frac{{d}^{2} \vec{W}}{d {t}^{2}} = ( {z}_{i o n} e ) {V}_{R F} Cos ( {ω}_{R F} t ) \vec{G} ( \vec{S} )$$

at any point along its path described by its secular motion. Since we consider \vec{G}(\vec{S}) to be constant on the scale of a single RF cycle, the solution of this differential equation is straightforward and may be given as:

$$\vec{W} ( t ; \vec{S} ) = - \frac{e / η}{( {m}_{i o n} / {z}_{i o n} ) {ω}_{R {F}^{2}}} {V}_{R F} \vec{G} ( \vec{S} ) Cos ( {ω}_{R F} t )$$

Thus, the amplitude of the sinusoidal “wiggle” motion, | [symbol] (t)|ₘₐₓ, is a function of the ion's secular displacement (proportional to magnitude of the electric field and aligned with the electric field):

$$\vert \vec{W} ( t ; \vec{S} ) {\vert}_{max} = - \frac{e / η}{( {m}_{i o n} / {z}_{i o n} ) {ω}_{R {F}^{2}}} {V}_{R F} \vert \vec{G} ( \vec{S} ) \vert$$

The velocity of the wiggle around the secular displacement, \vec{S}, may be given as:

$$\frac{d \vec{W} ( t ; \vec{S} )}{d t} = - \frac{e / η}{( {m}_{i o n} / {z}_{i o n} ) {ω}_{R F}} {V}_{R F} \vec{G} ( \vec{S} ) Sin ( {ω}_{R F} t )$$

where maximum ion speed of wiggle motion may be represented as:

$${\vert \frac{d \vec{W} ( t ; \vec{S} )}{dt} \vert}_{max} = \frac{e / η}{( {m}_{i o n} / {z}_{i o n} ) {ω}_{R F}} {V}_{R F} \vert \vec{G} ( \vec{S} ) \vert$$

Using these relationships, we can then derive an expression for the kinetic energy associated with this “wiggle” motion along the ion's secular trajectory as:

$$\begin{matrix}K {E}_{W} ( t ; \vec{S} ) = \frac{{( e / η )}^{2}}{{( {m}_{i o n} / {z}_{i o n} )}^{2} {ω}_{R {F}^{2}}} {V}_{R F}^{2} {\vert \vec{G} ( \vec{S} ) \vert}^{2} {Sin}^{2} ( {ω}_{R F} t ) \\ = \frac{1}{2} η {m}_{ion} \frac{{( e / η )}^{2}}{{( {m}_{i o n} / {z}_{i o n} )}^{2} {ω}_{R {F}^{2}}} {V}_{R F}^{2} {\vert \vec{G} ( \vec{S} ) \vert}^{2} ( \frac{1}{2} - \frac{1}{2} Cos ( 2 {ω}_{R F} t ) )\end{matrix} .$$

Therefore, the wiggle kinetic energy averaged over an RF cycle at any point along the ion's secular motion trajectory can be expressed as

$$〈 K {E}_{W} ( \vec{S} ) 〉 = \frac{1}{4} η {m}_{i o n} \frac{{( e / η )}^{2}}{{( {m}_{i o n} / {z}_{i o n} )}^{2} {ω}_{R {F}^{2}}} {V}_{R F}^{2} {\vert \vec{G} ( \vec{S} ) \vert}^{2} ,$$

and the maximum kinetic energy an ion may have within an RF cycle is twice this average value. In the Gerlich approach, this quantity behaves as a form of potential energy for the ion's secular motion, which Gerlich refers to as the effective potential. The sum of the kinetic energy associated with an ion's secular motion and the average kinetic energy of the ion over an RF cycle of the combined secular and wiggle motion, [symbol] KE [symbol] _{W}, is a constant, [symbol] KE [symbol] ₜₒₜₐₗ.

$$K {E}_{S} ( \vec{S} ) + 〈 K {E}_{W} ( \vec{S} ) 〉 = {〈 KE 〉}_{total}$$

$$and ;$$

$$\frac{1}{2} η {m}_{ion} {\vert \frac{d \vec{S}}{d t} \vert}^{2} + 〈 K {E}_{W} ( \vec{S} ) 〉 = {〈 KE 〉}_{total}$$

In the present representative calculations, we are treating the maximum kinetic energy an ion can attain at any point in time is 2 [symbol] KE [symbol] ₜₒₜₐₗ. Further, the equation of motion for the secular component of motion is given as:

$$η {m}_{i o n} \frac{{d}^{2} \vec{S}}{d {t}^{2}} = - ∇ 〈 K {E}_{W} ( \vec{S} ) 〉 .$$

Multiplying the quantity on the left-hand side by zᵢₒₙe/zᵢₒₙe and rearranging you get an equation of (secular) motion that looks exactly like one describing the motion of ion in an electrostatic potential:

$$η {m}_{i o n} \frac{{d}^{2} \vec{S}}{d {t}^{2}} = {z}_{i o n} e [ - ∇ \frac{〈 K {E}_{w} ( \vec{S} ) 〉}{{z}_{i o n} e} ] ,$$

where this “pseudo” electrostatic potential, Ψ(\vec{S}), given as:

$$\begin{matrix}Ψ ( \vec{S} ) = \frac{1}{4} \frac{η {m}_{i o n}}{{z}_{i o n} e} \frac{{( e / η )}^{2}}{{( {m}_{i o n} / {z}_{i o n} )}^{2} {ω}_{R {F}^{2}}} {V}_{R F}^{2} {\vert \vec{G} ( \vec{S} ) \vert}^{2} \\ = \frac{1}{4} \frac{( e / η )}{( {m}_{i o n} / {z}_{i o n} ) {ω}_{R {F}^{2}}} {V}_{R F}^{2} {\vert \vec{G} ( \vec{S} ) \vert}^{2}\end{matrix} .$$

The ions within the dissociation cell are driven by a time averaged force in the direction where they will experience the least wiggle motion—in the direction where they will experience the lowest electric field magnitude.

In our example RF 2D (linear) quadrupole ion trap, because of the symmetry of both the electrode structures and the trapping field imposing electrical fields applied to the device, the electric field from the RF trapping potential is zero along the central axis of device. If an ion has some oscillatory trajectory that brings it to the central axis:

$$\vec{S} = 0 \hat{ι} + 0 \hat{J} ,$$

the entirety of the kinetic energy is in its secular motion, as there is no electric field at the device's axis to induce wiggle motion. When the ion reaches a maximum in its secular motion (a turnaround point in its secular oscillation), all of its average kinetic energy will be in its wiggle motion.

Because of this, kinetic activation of the ions in the ion trap induces collisions with the neutral buffer gas molecules (e.g., helium atoms or other gases such as diatomic hydrogen, H, or diatomic nitrogen), which cause vibrational activation and lead to dissociation (i.e., ion trap type resonant CID), motion is imparted that is primarily on the x-axis of the device. Because the pressure (number density) of the atoms of the buffer (collision) gas is sufficiently high that over the time period of a few milliseconds after the ions that have been delivered to the device (e.g., injected axially into the device after their creation in an ion source), the collisions kinetically “cool” them such that their kinetic energies approach thermal energies and the ion's near-thermal-energy oscillations around the device axis are quite small compared to the device's internal dimensions. Maximum displacement for large molecules can be small fractions of a millimeter (mm). Thus, when the auxiliary potential is applied between the rod electrodes centered on the x-axis (by convention) at some auxiliary frequency, ω_{α}, that matches to one of the ion's characteristic frequencies for motion in the x-dimension, the ion's oscillations grow along the x-axis. While collisions with the background gas will couple some of this motion into the y-dimension (the other transverse dimension where the other pair of rod electrodes are centered) and the z-dimension (axially), this is a minor effect and the trajectories of the resonantly kinetically excited ions can be reasonably treated as one dimensional motion in the x-dimension. The spacing of the rod electrodes from the device's axis put a hard limit how far the ion trajectories can grow. For our example RF 2D (linear) quadrupole ion trap, the spacing of the rod electrodes from the device axis are uniform, and by convention are denoted as r₀. When an ion's x-dimension excursions reach either x=r₀ or x=−r₀ the ion will just graze the rod electrodes and be lost. Since such traps are symmetric about the y axis both physically in the imposed RF trapping potential essentially no odd order multipole components-only those introduced by small imprecisions in the mechanical construction and small), the magnitude of the maximum allowable magnitude of secular displacement in x, |s_{x,max}|, would therefore be:

$${s}_{x , max} = {r}_{0} - \vert {𝓌}_{x} ( {s}_{x , max} ) {\vert}_{max}$$

where |wₓ(s_{x,max})|ₘₐₓ is the amplitude of the wiggle motion at x=s_{x,max}.

Again because of the symmetry of the RF trapping potential, the spatially varying component of the trapping potential in the example RF 2D (linear) quadrupole ion trap can be expressed in polar coordinates as a two-dimensional (2D) multipole expansion:

$$g ( \vec{R} ) = g ( r , θ ) = {\sum }_{n = 0}^{∞} {{a}^{′}}_{n} \frac{{r}^{n}}{{r}_{0}^{n}} Cos n θ .$$

For convenience and by convention, each multipole term is normalized by

$${r}_{0}^{n}$$

and the newscaling constants, áₙ, relate to the scaling constants, aₙ in the expression of the generic 2D multipole expansion as:

$${{a}^{′}}_{n} = {a}_{n} {r}_{0}^{n}$$

With this normalization at displacements of ±r₀ on the x-axis, the surfaces of the x-rod electrodes, we have:

$$g ( {r}_{0} , 0 ) = g ( {r}_{0} , π ) = {\sum }_{n = 0}^{∞} {{a}^{′}}_{n} = 1$$

And correspondingly at displacements of ±r₀ on the y-axis the surfaces of the y-rod electrodes:

$$g ( {r}_{0} , \frac{3 π}{2} ) = g ( {r}_{0} , \frac{3 π}{2} ) = - {\sum }_{n = 0}^{∞} {{a}^{′}}_{n} = - 1$$

Because of the very specific symmetrically stretched geometry and application of RF voltage to the rod electrodes within the example RF 2D (linear) quadrupole ion trap, all of the scaling coefficients for the odd order coefficients (aₙ where n is odd) are all zero. Further the even order coefficients where n is a multiple of 4 are zero. So only the áₙ where n=2+4m for m=1, 2, 3 . . . are non-zero. Examples of multipole field determination compatible with the present teachings may be found in the scientific journal article by Philip M. Remes, John E. P. Syka, Viatcheslav V. Kovtoun, Jae C. Schwartz entitled “Insight into the Resonance Ejection Process during Mass Analysis through Simulations for Improved Linear Quadrupole Ion Trap Mass Spectrometer Performance” International Journal of Mass Spectrometry, Volume 377, 1 Feb. 2015, Pages 368-384, the entire contents of which is incorporated herein by reference. This allows the gradient of this multipole potential expansion along x-axis to be expressed as:

$$\vec{G} ( x \hat{ι} + 0 \hat{J} ) = {G}_{x} ( x , 0 ) \hat{ι} + 0 \hat{J}$$

$$where :$$

$${G}_{x} ( x , 0 ) = - {\sum }_{n = 1}^{∞} \frac{n {{a}^{′}}_{n}}{2 {{a}^{′}}_{2}} \frac{{x}^{n - 1}}{{r}_{0}^{n}} .$$

The first non-zero coefficient term in this power series expansion is the n=2 term which is the gradient of the quadrupolar portion of the expansion. We can rewrite this expression for the gradient along the x-axis as follows:

$${G}_{x} ( x , 0 ) = - ( 2 {{a}^{′}}_{2} \frac{x}{{r}_{0}^{2}} ) ( 1 + {\sum }_{n = 3}^{∞} \frac{n {{a}^{′}}_{n}}{2 {{a}^{′}}_{2}} \frac{{x}^{n - 2}}{{r}_{0}^{n - 2}} )$$

$$or as :$$

$${G}_{x} ( x , 0 ) = - ( 2 {{a}^{′}}_{2} \frac{x}{{r}_{0}^{2}} ) ( 1 + {ϵ}_{x} ( x ) )$$

$$Where :$$

$${ϵ}_{x} ( x ) = {\sum }_{n = 3}^{∞} n {{a}^{′}}_{n} \frac{{x}^{n - 2}}{{r}_{0}^{n - 2}} .$$

Expressed this way, the contributions of the higher order multipole potential terms to the gradient can be written as errors or deviations to the main quadrupole component of the field. It should be noted when if the trapping potential were purely quadrupolar then á₂=1 and all of the other áₙ are zero so ϵₓ(x)=0 and the expression simplifies (as it should) to that for the gradient along the x-axis of a purely quadrupolar potential.

Thus, methods and systems for ion trap type resonant collision induced dissociation (CID) on labile compounds to drive selective dissociation along desired dissociation pathways are disclosed. Specifically, the present disclosure includes, systems, methods, and computer readable media (CRM) configured to introduce ions of at least one labile compound with multiple dissociation pathways into a trapping area of a dissociation cell. The dissociation cell is configured to generate a substantially quadrupolar radio frequency electrostatic confinement potential in the ion trapping region. The kinetic energy of selected precursor ions is increased while a selective dissociation parameter is maintained to cause the precursor ions to selectively (i.e., preferentially) dissociate primarily to first-generation products without being ejected from the trapping area. The selective dissociation parameter is determined in part by the Mathieu q value at which the precursor ions are resonantly kinetically activated and controls the magnitude of the maximum velocity of the precursor ions. Over a fairly wide range of precursors m/z values (encompassing both a wide range of m/z and a wide range of elementary charges, z), the dissociation pathways for these precursors can be effectively controlled and the production of secondary dissociation products can be effectively suppressed by operating the ion trap such that the maximum magnitude of the precursor ion velocity, |v|ₘₐₓ, is maintained within empirically determined (or determinable) ranges for various buffer gases.

In some examples, the selective dissociation parameter is an estimate of the maximum magnitude of velocity, indicated herein as |v|ₘₐₓ, at any point along the trajectory of a by precursor ion within the confinement potential (e.g., quadrupolar or substantially quadrupolar RF confinement potential) of the dissociation cell during kinetic activation by application of the auxiliary AC potential such that the precursor ion trajectory does not exceed the confines of the electrode structure. In some particular architectures of ion traps, where the RF confinement field is wholly quadrupolar the maximum magnitude of velocity can be estimated as

$${\vert 𝓋 \vert}_{max} = \frac{\sqrt{3} {q}_{act} {ω}_{R F} {x}_{max}}{\sqrt{2 ( 1 + 0.90804633 {q}_{act} )}}$$

While in practical trapping devices the electrode shapes and their displacements from the device axis do introduce higher order multipole components to the trapping potentials, and these higher order field terms do influence the resonant kinetic process, the expression above still provides a reasonably accurate estimate of the maximum velocity magnitude. Similar determinations of maximum magnitude of velocity for excited labile ion species in 3D RF quadrupole ion traps (e.g., Paul traps) that are compatible with systems, methods, and computer-readable media of the present disclosure can be found at page 104 and also throughout the doctoral dissertation by Thomas Sebastian Neugebauer entitled “Dynamics of the Collision-Induced Dissociation Process in Quadrupole Ion Traps and its Application”, submitted Dec. 6, 2019 to the Faculty of Natural Sciences of the Friedrich-Alexander University, Erlangen-Nuremberg, the entire contents of which is incorporated herein by reference.

The most extreme excursions of the ion following a trajectory having this maximum magnitude of velocity would bring the precursor ions arbitrarily close to the electrodes establishing the confinement potential without actually impinging on the electrodes. Precursor ions will be confined at a specific Mathieu q for a given dissociation cell electrode structure having characteristic dimension, r₀, quadrupole potential scaling constant, á₂, the electrode structure and the associated applied RF potentials applied to the electrodes as defined by V_{RF} with angular frequency, ω_{RF}. In some example, the maximum magnitude of velocity for ions in the dissociation cell can depend upon a number of factors including V_{RF}, a voltage proportional to magnitudes of sinusoidal voltages applied to electrodes of said dissociation cell to establish said RF confinement potential, ORF, an angular frequency of the said RF confinement potential, q, the Mathieu activation value, and at least one characteristic dimension parameter, r₀, that defines how a physical electrode structure and geometry of said dissociation cell scales a magnitude of said RF confinement potential for a given magnitude of the applied RF confinement potential establishing voltages applied to electrodes of the dissociation cell.

During resonant kinetic activation, the maximum magnitude of velocity for precursor ions in the dissociation cell is defined as q_{act}, the Mathieu q value at which the precursors ions would be resonant with the auxiliary AC potential activation value, and the maximum allowed displacement the precursor ion may have and be retained in the ion trap (i.e., not impinge on an electrode or be ejected from the device) is xₘₐₓ. Here, we define the x dimension as the dimension in which the precursor ions are driven and resonantly kinetically activated by the auxiliary AC potential.

Operation of the dissociation cell such that the precursor ions upon kinetic activation cannot exceed the maximum magnitude of velocity taught herein for the conditions defined in order to induce labile precursor ion dissociation by preferred pathways and suppress generation of product ions from undesirable pathways (for Oligonucleotide deprotonated anions these would be ions resulting from two bond cleavages) can also depend upon characteristics of the buffer gas in the cell including a mass of the buffer gas and, to a lesser extent, on the pressure of the buffer gas in the collision cell.

The maximum magnitude of velocity can also depend upon the type of ionized molecules being analyzed. In some examples, an m/z-selected population of labile ions including deprotonated unmodified RNA ions, when using Helium (He) as the buffer gas, can be resonantly kinetically activated when confined in the RF potential of the dissociation cell such that the maximum magnitude of velocity for the precursor ion is within a range of 2870 to 3640 m/sec, or more preferably about 3070 m/sec. In another example, an m/z-selected population of labile ions including deprotonated unmodified RNA ions, when using diatomic Hydrogen (H₂) as the buffer gas, is resonantly kinetically activated when confined in the RF potential of the dissociation cell such that the maximum magnitude of velocity for the precursor ion is within a range of 2870 to 3640 m/sec, or more preferably about 3760 m/sec. In another example, an m/z-selected population of labile ions including deprotonated unmodified RNA ions, when using diatomic Nitrogen (N₂) as the buffer gas, is resonantly kinetically activated when confined in the RF potential of the dissociation cell such that the maximum magnitude of velocity for the precursor ions are within a range of 1480 to 1890 m/sec, or more preferably about 1680 m/sec. In another example, an m/z-selected population of labile ions including deprotonated unmodified RNA ions, when using Argon (Ar) as the buffer gas, is resonantly kinetically activated when confined in the RF potential of the dissociation cell such that the maximum magnitude of velocity for the precursor ions are within a range of 1270 to 1680 m/sec, or more preferably about 1460 m/sec.

In some examples, precursor ions are kinetically excited in both the x and y dimensions of the dissociation cell. The ranges given above for |v|ₘₐₓ will still apply. In some examples, the calculation of |v|ₘₐₓ for a given device with a particular set of trapping parameters and precursor m/z can be done by numerical simulation.

The above examples do not specify the buffer gas pressure or anything about the magnitudes of the AC auxiliary voltages applied to resonantly kinetically excite the precursor ions. Most commonly, a practical dissociation cell would be operated at pressures where the collisions only have a weak effect on the magnitude of the oscillations of the ions. The extrema of the ion trajectories of under-resonant kinetic excitation are primarily determined by the higher order components of the RF confinement field where the magnitude of the auxiliary field influences how frequently those extrema in displacements are reached and have a modest effect on the size of these extrema. The ranges given for |v|ₘₐₓ are applicable for such case. In many examples, an instrument operator can easily adjust the pressures of the buffer gas to the range of applicability. Further, the operator can adjust the magnitudes of the auxiliary AC voltage to effect sufficient production of precursor ions without ejection of the precursor ions. Such adjustments are well within the normal calibration/turning built into modern instruments.

By accounting for dissociation cell dimensions (such as r₀) and controlling the q-parameter at least by adjusting voltages on the electrodes of the dissociation cell, the velocity (and, therefore, kinetic energy) of the precursor ions selected for dissociation can be controlled to avoid both unwanted multiple-fragmentation pathways and to avoid colliding the precursor ions with the electrodes of the cell.

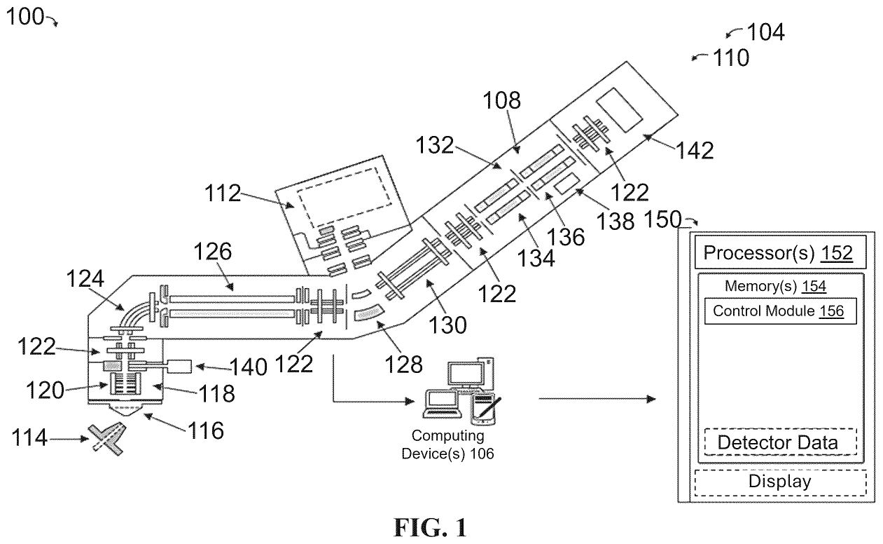

FIG. 1 illustrates example environments 100 for performing ion trap type resonance induced collision induced dissociation (CID) on labile compounds to drive selective dissociation along desired dissociation pathways. Specifically, FIG. 1 shows an example environment that includes an example mass spectrometer system 104 for complex mass spectrometry experiments and measurements using low Mathieu q dissociation of precursor ions, and computing devices 106 configured to control the operation of the mass spectrometer system 104 and/or perform post processing on detector data generated therefrom. It is noted that the present disclosure is not limited to environments that include mass spectrometers, and that in some embodiments the environments 100 may include a different type of system that is configured to manipulate and/or otherwise examine ions within an ion trap or may only include the computing devices 106. The environment 100 of FIG. 1 can be used in various combinations to generate both even and odd electron ions for low q dissociation. In various examples, product ions can be detected using a linear ion trap or an electrostatic trap at high resolution and mass accuracy.

The example mass spectrometer system 104 may be or include one or more different types of mass spectrometers known in the art that comprise dissociation cell 108 configured to allow for the dissociation of precursor ions (e.g., RF quadrupole ion trap devices, etc.). In various examples, dissociation cells compatible with the systems and methods taught herein include linear quadrupole traps, rf Paul traps, two-dimensional (2D) ion traps, three-dimensional (3D) ion traps, toroidal ion traps, and cells with curved axes.

FIG. 1 shows the example mass spectrometer system(s) 104 as being a hybrid mass spectrometer 110, comprising more than one type of mass analyzer. Specifically, the mass spectrometer system 110 includes a dissociation cell 108 as well as an optional electrostatic trap mass analyzer 112 (e.g., ORBITRAP™ analyzer). However, it is understood that different combinations of mass analyzers are desirous for different applications, and thus according to the present disclosure example mass spectrometer systems 104 may include a fewer or greater number of mass analyzers and/or comprise different combinations of mass analyzers.

In operation of the example mass spectrometer system 110, an ion source 114 (such as an electrospray ion source) provides ions of a sample to be analyzed to an aperture of a heated ion transfer tube 116, at which point the ions enter into a first vacuum chamber 118. After entry, the ions are captured and focused into a tight beam by an ion collimating device 120 (e.g., a stacked-ring ion guide, an ion lens, an ion funnel, etc.). The example mass spectrometer 110 is further shows as including a plurality of ion optical transfer components 122 that are configured to allow ions to pass between intermediate-vacuum regions of the mass spectrometer during travel. Example spectrometer 110 is illustrated as including a curved beam guide 124 that separates most remaining neutral molecules and undesirable ion clusters (e.g., solvated ions, environmental contaminants, etc.) from the ion beam. For example, neutral molecules and ion clusters follow a straight-line path whereas the paths of ions of interest are bent around the ninety-degree turn of the curved beam guide 124, thereby producing the separation.

A quadrupole mass filter 126 of the mass spectrometer system 110 is used in its conventional sense as a tunable mass filter so as to pass ions only within a selected m/z range. A subsequent ion optical transfer component 122 delivers the filtered ions to a curved ion trap (“C-trap”) component 128. The C-trap 128 is able to transfer ions along a pathway between the quadrupole mass filter 126 and the ion trap mass analyzer 108. The C-trap 128 also has the capability to temporarily collect and store a population of ions and then deliver the ions, as a pulse or packet, into the mass analyzer 112.

FIG. 1 further shows a multipole ion guide 130 and an optical transfer component 122 as serving to guide ions between the C-trap 128 and the ion trap mass analyzer 108. The multipole ion guide 130 may provide temporary ion storage capability such that ions produced in a first processing step of an analysis method can be later retrieved for processing in a subsequent step. The multipole ion guide 130 may also serve as a fragmentation cell and ion trap (i.e., an ion routing multipole). Various ion optics along the pathway between the C-trap 128 and the ion trap mass analyzer 108 may be controllable such that ions may be transferred in either direction, depending upon the sequence of ion processing steps required in a particular analysis method.

The ion trap mass analyzer 108 is illustrated in FIG. 1 as being a dual-pressure linear ion trap 132 (i.e., a two-dimensional trap) comprising a high-pressure linear trap cell 134 and a low-pressure linear trap cell 136, the two cells being positioned adjacent to one another and separated by a plate lens having a small aperture that permits ion transfer between the two cells and that also acts as a pumping restriction that allows different pressures to be maintained in the two traps. However, a person having skill in the art would understand that other types of ion traps 108 are capable of performing the induced collision induced dissociation (CID) on labile compounds to drive selective dissociation along desired dissociation pathways as described in the present disclosure, including those described in the Enumerated Paragraphs, and using any of the parameters described herein or which are widely understood by persons having skill in the art as being part of performing such methods.

The environment of the high-pressure cell 134 favors ion trapping, ion cooling, ion fragmentation by either collision-induced dissociation or pulsed-q dissociation, ion/ion reactions by either electron transfer dissociation or proton-transfer reactions, and some types of photon activation, such as ultraviolet photo dissociation (UVPD). The environment of the low-pressure cell 136 favors analytical scanning with high resolving power and mass accuracy. The ion trap 108 further is shown as including an ion detector 138 (e.g., a dual-dynode ion detector).

The use of either electron transfer dissociation or a proton transfer reaction, within a mass analysis method, requires the capability of performing controlled ion-ion reactions within a mass spectrometer. Ion-ion reactions, in turn, require the capabilities of generating reagent ions, and of causing the reagent ions to mix with sample ions. The example mass spectrometer system 110 is depicted as including a reagent-ion source 140 disposed between the stacked-ring ion guide and the curved beam guide 124. However, within the present disclosure one or more additional reagent-ion sources may be included in an example mass spectrometer system 104. FIG. 1 further illustrates the example mass spectrometer 110 as including one or more additional components 142. Such additional components may include various combinations of one or more ion guides, ion traps, lenses, detectors, reagent ion sources, etc. A person having skill in the art would appreciate that example mass spectrometer 110 is merely an example configuration of a system capable of enabling/performing the system and methods for low Mathieu q dissociation of precursor ions disclosed herein.

The environment 100 is also shown as including one or more computing device(s) 106. Those skilled in the art will appreciate that the computing devices 106 depicted in FIG. 1 are merely illustrative and are not intended to limit the scope of the present disclosure. The computing system and devices may include any combination of hardware or software that can perform the indicated functions, including computers, network devices, internet appliances, PDAs, wireless phones, controllers, oscilloscopes, amplifiers, etc. The computing devices 106 may also be connected to other devices that are not illustrated, or instead may operate as a stand-alone system,

It is also noted that one or more of the computing device(s) 106 may be a component of the example mass spectrometers 104, may be a separate device from the example mass spectrometers 104 which is in communication with the example mass spectrometers 104 via a network communication interface, or a combination thereof. For example, mass spectrometers 104 may include a first computing device 106 that is a component portion of the example mass spectrometers 104, and which acts as a controller that drives the operation of the example mass spectrometers 104 (e.g., adjust the scanning location on the sample by operating the scan coils, etc.). In such an embodiment the example mass spectrometers 104 may also include a second computing device 106 that is a desktop computer separate from the example system(s) 104, and which is executable to process data received from the detector system 138 to generate representations of the spectra based on the detector data (e.g., chromatograms, extracted ion current (EIC) profiles, etc.) and/or perform other types of analysis or post-processing of the detector data. The computing devices 106 may further be configured to receive user selections via a keyboard, mouse, touchpad, touchscreen, wireless devices, other user interface, etc.

Additionally, the computing device(s) 106 are configured to control the example mass spectrometers 104 to allow for the performance a mass spectrometry analysis on a sample. For example, one or more user selections, an automation program, or a combination thereof may allow the computing devices 106 to cause mass spectrometers 104 and/or components thereof to perform any of the methods described in the present disclosure, including those described in the Enumerated Paragraphs, and using any of the parameters described herein or which are widely understood by persons having skill in the art as being part of performing such methods. User selections, an automation program, or a combination thereof may then cause the computing devices 106 to generate analyze detector data from the mass spectrometers 104 relating to a sample, and/or create one or more chromatograms associated with the performed mass spectroscopy analysis of the samples.

The computing devices 106 may then determine characteristic information about the product ions created by the process of performing resonance induced collision induced dissociation (CID) on labile compounds to drive selective dissociation along desired dissociation pathways according to the present disclosure. For example, the computing devices 106 may determine the identity and/or sequences of oligonucleotides in the sample having at least 20mer, 30mer, 50mer, 100mer, or greater than 200mer. In various embodiments, the computing devices 106 may use scripted algorithms, AI/ML generated algorithms, or a combination thereof to process the detector data derived using the methods and system of the present disclosure to identify, characterize, study, and/or sequence labile macromolecules that were previously unable to be reliably sequences in the state of the art.

FIG. 1 further includes a schematic diagram illustrating an example computing architecture 150 of the computing devices 105. Example computing architecture 150 illustrates additional details of hardware and software components that can be used to implement the techniques described in the present disclosure. Persons having skill in the art would understand that the computing architecture 150 may be implemented in a single computing device 106 or may be implemented across multiple computing devices. For example, individual modules and/or data constructs depicted in computing architecture 150 may be executed by and/or stored on different computing devices 106. In this way, different process steps of the inventive methods disclosed herein may be executed and/or performed by separate computing devices 106 and in various orders within the scope of the present disclosure. In other words, the functionality provided by the illustrated components may in some implementations be combined in fewer components or distributed in additional components. Similarly, in some implementations, the functionality of some of the illustrated components may not be provided and/or other additional functionality may be available.

In the example computing architecture 150, the computing device includes one or more processors 152 and memory 154 communicatively coupled to the one or more processors 152. While not intended to be limiting, example computing architecture 150 is shown as including a control module 166 stored in the memory 154. As used herein, the term “module” is intended to represent example divisions of executable instructions for purposes of discussion and is not intended to represent any type of requirement or required method, manner, or organization. Accordingly, while various “modules” are described, their functionality and/or similar functionality could be arranged differently (e.g., combined into a fewer number of modules, broken into a larger number of modules, etc.). Further, while certain functions and modules are described herein as being implemented by software and/or firmware executable on a processor, in other instances, any or all of modules can be implemented in whole or in part by hardware (e.g., a specialized processing unit, etc.) to execute the described functions. As discussed above in various implementations, the modules described herein in association with the example computing architecture 150 can be executed across multiple computing devices 106.

The control module 168 can be executable by the processors 152 to cause a computing device 106 and/or example mass spectrometers 104 to take one or more actions and/or perform functions or maintenance of the systems. In some embodiments, the control module 168 may cause the example mass spectrometers 104 to perform a mass spectrometry analysis on a sample. More specifically, according to the present disclosure, the example control module 168 can be executable to cause mass spectrometers 104 and/or components thereof to perform any of the methods described in the present disclosure, including those described in the Enumerated Paragraphs, and using any of the parameters described herein or which are widely understood by persons having skill in the art as being part of performing such methods.

As discussed above, the computing devices 106 include one or more processors 152 configured to execute instructions, applications, or programs stored in a memory(s) 154 accessible to the one or more processors. In some examples, the one or more processors 152 may include hardware processors that include, without limitation, a hardware central processing unit (CPU), a graphics processing unit (GPU), and so on. While in many instances the techniques are described herein as being performed by the one or more processors 152, in some instances the techniques may be implemented by one or more hardware logic components, such as a field programmable gate array (FPGA), a complex programmable logic device (CPLD), an application specific integrated circuit (ASIC), a system-on-chip (SoC), or a combination thereof.

The memories 154 accessible to the one or more processors 152 are examples of computer-readable media. Computer-readable media may include two types of computer-readable media, namely computer storage media and communication media. Computer storage media may include volatile and non-volatile, removable, and non-removable media implemented in any method or technology for storage of information, such as computer readable instructions, data structures, program modules, or other data. Computer storage media includes, but is not limited to, random access memory (RAM), read-only memory (ROM), erasable programmable read only memory (EEPROM), flash memory or other memory technology, compact disc read-only memory (CD-ROM), digital versatile disk (DVD), or other optical storage, magnetic cassettes, magnetic tape, magnetic disk storage or other magnetic storage devices, or any other non-transmission medium that may be used to store the desired information and which may be accessed by a computing device. In general, computer storage media may include computer executable instructions that, when executed by one or more processing units, cause various functions and/or operations described herein to be performed. In contrast, communication media embodies computer-readable instructions, data structures, program modules, or other data in a modulated data signal, such as a carrier wave, or other transmission mechanism. As defined herein, computer storage media does not include communication media.

Those skilled in the art will also appreciate that items or portions thereof may be transferred between memory 154 and other storage devices for purposes of memory management and data integrity. Alternatively, in other implementations, some or all the software components may execute in memory on another device and communicate with the computing devices 106. Some or all of the system components or data structures may also be stored (e.g., as instructions or structured data) on a non-transitory, computer accessible medium or a portable article to be read by an appropriate drive, various examples of which are described above. In some implementations, instructions stored on a computer-accessible medium separate from the computing devices 106 may be transmitted to the computing devices 106 via transmission media or signals such as electrical, electromagnetic, or digital signals, conveyed via a communication medium such as a wireless link. Various implementations may further include receiving, sending, or storing instructions and/or data implemented in accordance with the foregoing description upon a computer-accessible medium.

### III. Method

### A. Instrumentation and Method of Using

The field of mass spectrometry offers a variety of tools and techniques for analyzing ions based upon the mass-to-charge (m/z) ratios of such ions. For example, a mass spectrometry system can include various devices for producing precursor ions for analysis, isolating the precursor ions of interest, fragmenting the precursor ions into product ions, and/or mass analyzing the precursor ions and/or product ions. In particular, in various examples, a mass spectrometry system can include a fragmentation device and a mass analyzer downstream of the fragmentation device. Such a configuration may be described as a tandem mass spectrometry system. In such examples, the fragmentation device can receive precursor ions derived from an ion source that have been subsequently isolated using applied waveforms to the ion trap or passed through a quadrupole mass filter or similar device. The precursor ions are fragmented into product ions, followed by mass analysis either directly in the linear ion trap or by moving the ions to the electrostatic ion trap (such as an Orbitrap™ mass analyzer). The product ions are analyzed based upon their respective m/z ratios. Measuring the relative abundance of the product ions based upon the m/z ratios thereof can enable identification of various properties of the precursor ions, such as a molecular composition and/or sequence of a polymeric material, such as a nucleic acid.

In this manner, tandem mass spectrometry can serve as a valuable tool for analyzing macromolecular structures and complexes, such as those comprising oligonucleotides, including performing both de novo sequencing of confirmational sequencing of such oligonucleotides. In the absence of a reference sequence to which the product ions can be compared, de novo sequencing relies upon generating mass spectrometry spectra from which the identity and sequence of the product ions can be accurately determined. In the case of large oligonucleotides, high-energy fragmentation of the precursor ions typically produces a wide variety of product ions representing overlapping subsets of the isotopic distribution, thereby producing a mass spectrum that is difficult if not impossible to interpret. This complexity increases as the nucleotide length increases. Accordingly, one aspect of the present invention concerns de novo sequencing of oligonucleotides by fragmenting the precursor ions in a controlled and/or predictable manner, such as by preferentially cleaving the precursor ions at well-defined labile bonds. Embodiments of the system and method disclosed herein fragmenting oligonucleotide precursor ions into product ions that provide oligonucleotide sequence information in a controlled and/or predictable manner.

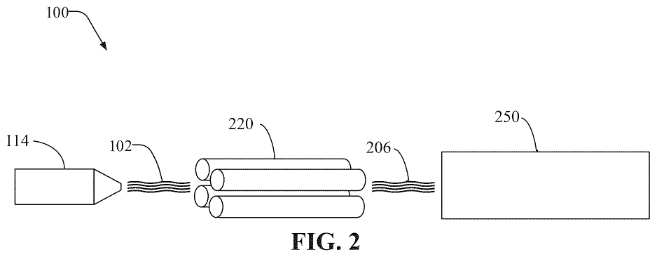

One embodiment of an exemplary mass spectrometry system 100 according to the present disclosure is illustrated schematically by FIG. 2. As shown in FIG. 2, the mass spectrometry system 100 can include an ion source 114 for generating precursor ions 202 comprising oligonucleotide ions, a fragmentation device 220 positioned downstream of the ion source 114 (similar to the ion guiding device 130 acting as a fragmentation device in FIG. 1), and a mass analyzer 250 positioned downstream of the fragmentation device 220. The fragmentation device 220 can be configured to receive the precursor ions 102 and to collide the precursor ions 102 with a buffer gas disposed therein, resulting in fragmentation of the precursor ions 102 into product ions 206. The mass analyzer 250 can be configured to mass analyze the product ions 206, such as to produce a mass spectrum of the product ions 206. In this manner, the mass spectrometry system 100 is configured to perform collision-induced dissociation (CID) of the precursor ions 102 to produce the product ions 206.

The ion source 114 can be configured to generate the precursor ions 102 in any suitable manner. In particular, the ion source 114 can employ a soft ionization technique that limits and/or minimizes the extent to which the precursor ions 102 are fragmented prior to entering the fragmentation device 220. As more specific examples, the ion source 114 can include and/or be an electrospray (ESI) ion source (e.g., a nano ESI ion source), a matrix-assisted laser desorption/ionization (MALDI) ion source, a laser spray ion source, and/or an inlet ionization source.

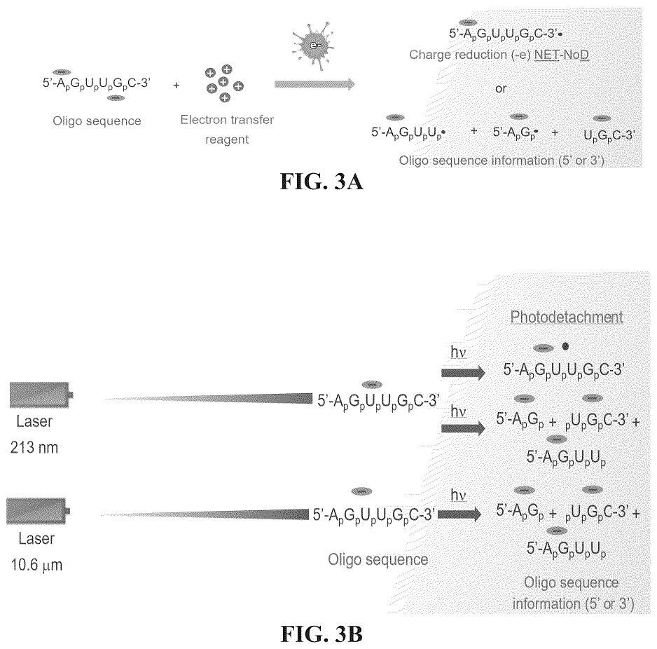

FIG. 3A is a schematic drawing illustrating electron transfer dissociation, whereby an oligomer is reacted with an electron transfer reagent to produce either a charge reduction event (e.g., without fragmentation) or a fragmented precursor oligomer ion. Moreover, oligonucleotides fragment to produce product ions that can be mass analyzed to provide sequence information in a 5′ and/or a 3′ direction. Fluoranthene is one example of an electron transfer reagent that can be used for oligonucleotide sequencing.

FIG. 3B is a schematic demonstrating using ultraviolet photodetachment (PD-UV) to produce ions suitable for mass analysis. The product ions produced via this process increase in charge state by 1 due to the loss of an electron. A person of ordinary skill in the art will appreciate that various different UV wavelengths can be used to perform PD-UV, although certain disclosed embodiments contemplate using a wavelength of about 213 nm fragmented ions suitable for mass analysis. In another exemplary aspect, the method comprises using infrared multiphoton dissociation (IRMPD), wherein the wavelength in an exemplary embodiment was 10.6 μm, to produce fragment ions for mass analysis and ultimately determining sequence information in the 5′ and/or 3′ direction. In some examples, the precursor ions can be generated and isolated using the instrument described in FIG. 1. The precursor ions 102 can be generated with a selected charge state in some examples.

Mass analyzer 250 can be configured to analyze product ions 206 in any suitable manner. For example, the mass analyzer 250 can be configured to preferentially trap and/or expel the product ions 206 based upon a mass-to-charge ratio thereof. Additionally or alternatively, the mass analyzer 250 can be configured to generate a mass spectrum that indicates the relative prevalence of the product ions 206 as a function of the mass-to-charge ratios thereof. In various examples, the mass analyzer 250 can include and/or be an electrostatic trap (e.g., an orbital trap mass analyzer), a time of flight (TOF) quadrupole mass analyzer, and/or an FT-ICR mass analyzer.

The fragmentation device 220 can have any of a variety of configurations for fragmenting the precursor ions 102 as described herein. In particular, the present disclosure generally relates to examples in which the fragmentation device 220 includes and/or is a quadrupole-based linear ion trap; however, it is to be understood that other geometries and/or configurations of fragmentation devices can be used.

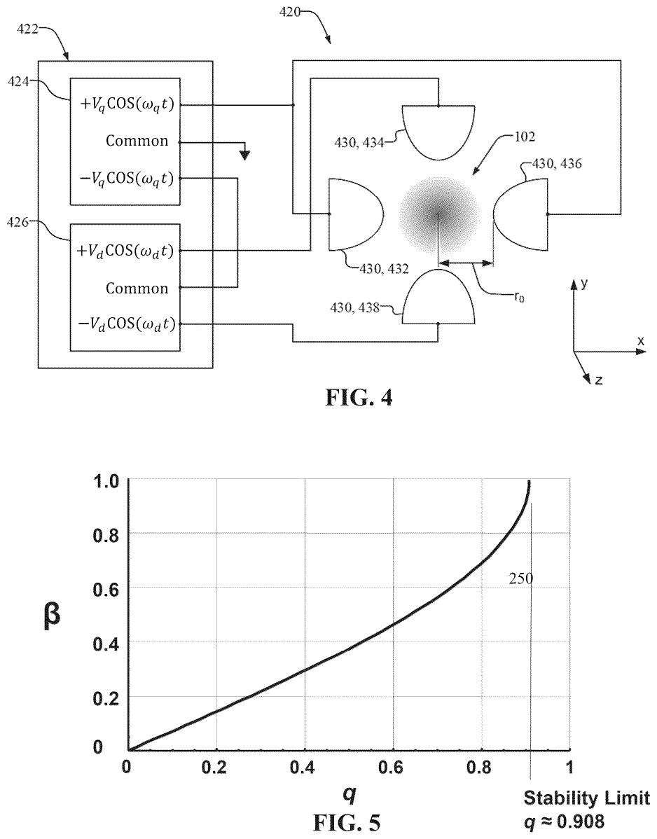

FIG. 4 provides a schematic view of one exemplary fragmentation device 420 of FIGS. 1 and 2 and further illustrates associated control components. With reference to FIG. 4, the fragmentation device 420 can include four electrodes 430 and an RF power supply 422 configured to apply voltages to the electrodes. Specifically, the RF power supply 422 is configured to apply a trapping voltage waveform to a first subset of the electrodes 430 to generate an RF quadrupole electric field and to apply an auxiliary voltage waveform to a second subset of the electrodes 430 to generate an applied auxiliary electric field. The RF power supply 422 can include a trapping voltage waveform generator 424 configured to generate the trapping voltage waveform and an auxiliary voltage waveform generator 426 configured to generate the auxiliary voltage waveform. This is not required of all aspects, however, and it is within the scope of the present disclosure that the RF power supply 422 can generate the trapping voltage waveform and the auxiliary voltage waveform in any suitable manner. The plurality of electrodes 430 includes a first pair of opposed electrodes 432, 436 spaced apart along a first axis (e.g., the x-axis shown in FIG. 4) and a second pair of opposed electrodes 434, 438 spaced apart along a second axis perpendicular to the first axis (e.g., the y-axis shown in FIG. 4). In this aspect, the fragmentation device 420 can be configured to generate an RF quadrupole electric field by applying the trapping voltage waveform to each electrode 432, 436 of the first pair of opposed electrodes with a first phase and applying the trapping voltage waveform to each electrode 434, 438 of the second pair of opposed electrodes with a second phase that is 180 degrees out of phase with the first phase.

The trapping voltage waveform can be characterized by a trapping voltage amplitude V_{RF} and a trapping field frequency ω_{RF}. Accordingly, the voltage applied to each electrode 430 of the first pair of opposed electrodes can be expressed as V_{1,3}(t)=V_{RF} cos(ω_{RF}t), while the voltage applied to each electrode 430 of the second pair of opposed electrodes can be expressed as V_{2,4}(t)=−V_{RF} COS (ω_{RF}t). In such expressions, the numerical subscripts on each voltage denote the electrodes 430 to which the voltage is applied, with the indices 1, 2, 3, and 4 representing the electrodes 432, 434, 436, and 438, respectively. The resulting RF quadrupole electric field exerts a force upon an ion positioned between the electrodes 430 that accelerates the ion toward a central axis of the fragmentation device 420 (e.g., the z-axis in FIG. 4), thus trapping the ion within the fragmentation device 420 in two dimensions (e.g., within a region of the x-y plane in FIG. 4).

The RF quadrupole electric field accelerates the ions within the fragmentation device 420 in such a manner that the ions oscillate relative to the central axis of the fragmentation device 420 with a “secular motion” that can be characterized by a secular amplitude and a secular frequency. In particular, in the absence of the applied auxiliary AC electric field (auxiliary AC waveform), and in the absence of a DC component to the trapping voltage waveform, the equations of motion of an ion positioned in an ideal purely quadrupole field take the form of the Mathieu Equation where there is no static part of the time variant coefficient of u:

$$\frac{{d}^{2} u}{d {ξ}^{2}} - 2 {q}_{u} cos ( 2 ξ ) u = 0 ,$$

$$where :$$

$${q}_{u} = \frac{4 ( e / η ) {{a}^{′}}_{2} {V}_{R F}}{( m / z ) {r}_{0}^{2} {ω}_{R F}^{2}} ,$$

In this equation u denotes a selected dimension (e.g., the x or y dimension), and ξ is a normalized time unit defined such that the angular frequency of the RF quadrupole trapping field is equal to 2. The quantity (m/z) denotes the mass-to-charge ratio of the trapped ion (as discussed above), while r₀ denotes a radius (characteristic dimension) of the fragmentation device 420. For example, and as shown in FIG. 4, r₀ can represent a minimum distance from the central axis of the fragmentation device 420 to any of the electrodes 430 thereof. Since the fragmentation device 420 is a RF-only linear quadrupole ion trap, the equations of motion in the x and y dimensions in Mathieu form are related in that

$${q}_{u} = - {q}_{y}$$

Reflecting the antiphase relationship between the relationship between action of the RF quadrupole potential in the x and y dimensions.
Solutions to such Mathieu type equations of motion in the regime of interest are of the form

$${M}_{sin} ( ζ ; q ) = {\sum }_{n = - ∞}^{∞} {C}_{2 n} sin ( ( β + 2 n ) ξ ) ;$$

$${M}_{cos} ( ξ ; q ) = {\sum }_{n = - ∞}^{∞} {C}_{2 n} cos ( ( β + 2 n ) ξ ) ,$$

where β corresponds to the primary resonant frequency of the ion's motion in the dimension of interest. Both β and the weighting coefficients, C₂ₙ, are wholly functions of qᵤ. The unforced motion of any ion in the dimension of interest will be some superposition of these two solutions. In particular, β is related to the secular ion frequency ωᵢₒₙ in the real frequency domain as ωᵢₒₙ=βω_{q}/2. The quantity qᵤ may be described as a trapping parameter representing the normalized effective intensity of the confining field of the trapped ion, while the quantity β may be described as a normalized characteristic frequency of motion the ion. The relationship between the quantities β and q can at least partially characterize a stability with which an ion can be trapped within the fragmentation device 420. For example, FIG. 5 illustrates a diagram depicting the manner in which β depends upon q. As shown in FIG. 5, for values of q less than about 0.5, β depends upon q roughly linearly, with β≈q/\sqrt{2}. At higher values of q, the dependence of β upon q becomes increasingly nonlinear, with β approaching 1 as q values approach a stability limit of qₗᵢₘ≈0.908. Above that stability limit, one of the solutions becomes an exponentially growing oscillation—the motion is unstable.

For a given experimental configuration (e.g., for given values of V_{RF}, ω_{RF}, and r₀), ions can be stably trapped only if the m/z ratios of such ions can yield a trapping parameter q value that is below the stability limit. Such ions are then constrained to an orbit around the central axis of the fragmentation device 420, where the trapped ions form a “cloud” of ions in constant secular motion. Conversely, ions that are characterized by a trapping parameter q above the stability limit will have an unstable trajectory that is not confined within the fragmentation device 420.

With the precursor ions 102 trapped within the fragmentation device 420 by the RF quadrupole electric field, the precursor ions 102 further can be accelerated by the applied auxiliary electric field to collide with buffer gas molecules to fragment the precursor ions 102 and form productions 206. The applied auxiliary electric field can produce growth in the amplitude of motion of the precursor ions 102 that result in inelastic collisions between the precursor ions 102 and the buffer gas. Such collisions convert the kinetic energy of the precursor ions 102 into vibrational energy of the precursor ions 102. Increased vibrational energy induces bond cleavage in the precursor ions 102 to produce product ions 206 in a controlled manner.

Without being bound by a theory of operation, the auxiliary voltage waveform can be characterized by an auxiliary voltage amplitude V_{d} and an auxiliary field frequency ω_{d}, and can take the form Vₐᵤₓ(t)=V_{d} cos(ω_{d}t). As discussed above with reference to FIG. 4, the applied auxiliary electric field can be produced by applying the auxiliary voltage waveform to each electrode 130 of the second subset of electrodes with opposite respective signs. Again with reference to FIG. 4, the second subset of electrodes 130 includes the electrodes 134, 138 of the second pair of opposed electrodes. Thus, for example, the total voltage applied to the electrode 134 can be expressed as V₂(t)=V_{2,4}(t)+Vₐᵤₓ(t), while the total voltage applied to the electrode 138 can be expressed as V₄(t)=V_{2,4}(t)−Vₐᵤₓ(t), where V_{2,4}(t) is as defined above with respect to the trapping voltage waveform.

The applied auxiliary electric field produced by the auxiliary voltage waveform can include and/or be a dipole electric field that further accelerates ions within the fragmentation device 420 to induce ion micromotion. In particular, with the electric field within the fragmentation device 420 representing a superposition of the RF quadruple electric field and the applied auxiliary electric field, the equations of motion of the trapped ions take the form of the solutions to the modified Mathieu Equation:

$$\frac{{d}^{2} u}{d {ξ}^{2}} - 2 q cos ( 2 ξ ) u = P cos ( αξ ) ,$$

$$where$$

$$P ∼ \frac{( e / η ) {V}_{d}}{( m / z ) {r}_{0} {ω}_{q}^{2}}$$

$$and$$

$$α = \frac{{ω}_{d}}{{ω}_{q} / 2} .$$

The term P cos(αξ) may be understood as a driving force for inducing growth in the oscillatory motion of the trapped precursor ions 102 that collides the precursor ions 102 with the buffer gas. In particular, with the trapped precursor ions 102 characterized by a given value of the trapping parameter q, the auxiliary electric field can be applied with an auxiliary field frequency ω_{d} corresponding to this value of q to resonantly excite the ions to exhibit the micromotion. Therefore, the applied auxiliary electric field can cause the precursor ions 102 to collide with the precursor gas sufficient to result in fragmentation and to produce ions 206 only when a relationship between the auxiliary field frequency ω_{d} and the normalized characteristic frequency β satisfies a resonance condition.

Owing to the relationship between the normalized characteristic frequency β and the trapping parameter q, this resonance condition also can be described as a correspondence between the auxiliary field frequency ω_{d} and the parameters upon which the trapping parameter q is based. Thus, in practice, this resonance condition may be achieved by configuring and/or varying any of a variety of instrument and/or experimental parameters.

In particular aspects disclosed herein, the resonance condition can be achieved via “q-scanning,” in which the auxiliary field frequency ω_{d} is held constant and one, or more, other parameters is varied to sweep the trapping parameter q through a range of such values. In particular, the selected auxiliary field frequency ω_{d} may be described as corresponding to a target trapping parameter qt at which the resonance condition is met, and one or more experimental parameters may be varied to sweep the trapping parameter q through a q sweep range that includes qt. Stated differently, the q sweep range can be defined by a maximum value qₘₐₓ and a minimum value qₘᵢₙ such that qₘᵢₙ<qₜ<qₘₐₓ.

In practice, q-scanning may be most readily accomplished by varying the trapping voltage amplitude V_{q} to vary the trapping parameter q. This is not required, however, and it also is within the scope of the present disclosure that the trapping field frequency ω_{q} may be varied to sweep the trapping parameter through the q sweep range.

In some aspects, q-scanning is performed by repeatedly sweeping the trapping parameter q through the q sweep range over a scan period that is approximately 5-10 milliseconds (ms) per scan. Additionally, or alternatively, q-scanning can be performed by sweeping the trapping parameter q through the q sweep range from the maximum value qₘₐₓ to the minimum value qₘᵢₙ, or vice-versa.

As another example, a desired resonance condition can be achieved via “secular scanning,” in which the trapping parameter is fixed at a value qₜ and the auxiliary field frequency ω_{d} is swept through an auxiliary field frequency range that includes the secular frequency ωᵢₒₙ. Stated differently, the auxiliary field frequency range can be defined by a maximum value ω_{d,max} and a minimum value ω_{d,min} such that ω_{d,min}<ωᵢₒₙ<ω_{d,max}.

In some aspects, secular scanning is performed by repeatedly sweeping the auxiliary field frequency through the auxiliary field frequency range over a scan period that is approximately 5-10 ms per scan. Additionally or alternatively, secular scanning can be performed by sweeping the auxiliary field frequency ω_{d} through the auxiliary field frequency range from the maximum value ω_{d,max} to the minimum value ω_{d,min}, or vice-versa.

The value qₜ of the trapping parameter at resonance can be selected based on any of a variety of considerations. For example, selection of qₜ can place constraints on a range of product ions 206 that will remain stably trapped by the fragmentation device 420 after fragmentation. Because the product ions 206 generally are characterized by a different mass-to-charge ratio m/z than the precursor ions 102, the product ions 206 produced while the precursor ions 102 are characterized by the trapping parameter qₜ are in turn characterized by a trapping parameter that is shifted away from qₜ. In particular, product ions 206 with smaller m/z ratios than the precursor ions 102 will be characterized by larger trapping parameters q relative to that of the precursor ions 102. Accordingly, it is desirable that qₜ be sufficiently low that the product ions 206 can exhibit a range of m/z ratios corresponding to trapping parameters q that remain below the stability limit qₗᵢₘ discussed above with reference to FIG. 5.

Another consideration for optimizing qₜ arises from the direct proportionality between q and the trapping voltage amplitude V_{q}, which in turn can be related to an energy with which the trapped precursor ions 102 collide with the buffer gas. In this manner, increasing values of qₜ can be associated with increasing collision energy, resulting in fragmentation of the precursor ions 102 with greater efficiency.

In some aspects, selection of the value of qₜ represents a compromise between qₜ being sufficiently high to yield sufficient collision energy for effective fragmentation of the precursor ions 102 and qₜ being sufficiently low to allow for a wide range of fragment ion masses to remain stably trapped within the fragmentation device 420.

In particular aspects disclosed herein, performing precursor ion fragmentation at a trapping parameter value of qₜ=0.25 offers an effective compromise between these considerations. In other examples, such as when fragmenting oligonucleotide ions, using a trapping parameter of qₜ=0.25 generates product ions 206 comprising an undesirably high abundance of second (or higher) generation dissociation ion products. By contrast, fragmenting the precursor ions 102 at a lower trapping parameter value of qₜ≈0.15 can suppress the generation of such undesirable dissociation ion products.

As discussed above, realization of a specific qₜ value characterizing the precursor ions 102 undergoing fragmentation can be achieved by appropriate selection ng of various experimental parameters. As an example, fragmenting oligonucleotide ions characterized by a trapping parameter of qₜ=0.25 may correspond to a (linear) auxiliary field frequency f_{d}=ω_{d}/2π≈144 kHz and r₀≈4.75 millimeters (mm). As another example, fragmenting oligonucleotide precursor ions 102 characterized by a trapping parameter of qₜ=0.15 may correspond to a (linear) auxiliary field frequency f_{d}=ω_{d}/2π≈86 kHz and r₀≈4.75 millimeters (mm).

Such examples are not required, however, and it also is within the scope of the present disclosure that any suitable combination of experimental parameters may be used to yield any suitable trapping parameter qₜ. As examples, precursor ion fragmentation may be performed with the precursor ions characterized by a trapping parameter qₜ as disclosed herein.

In examples in which the resonance condition that fragments precursor ions are achieved via q-scanning, the q-scanning can be performed in any suitable manner. For example, the q-scanning can include sweeping the trapping parameter q through a q sweep range that is characterized by a maximum value qₘₐₓ and a minimum value qₘᵢₙ that each differ from the target value qₜ as disclosed herein. Additionally or alternatively, q-scanning can include sweeping the trapping parameter q over a scan period as disclosed herein.

In examples in which the resonance condition that fragments precursor ions are achieved via secular scanning, the secular scanning can be performed in any suitable manner. For example, the secular scanning can include sweeping the auxiliary field frequency ω_{d} through an auxiliary field frequency range that is characterized by a maximum value ω_{d,max} and a minimum value ω_{d,min} that each differ from the target secular frequency ωᵢₒₙ as disclosed herein.

In various examples, fragmenting precursor ions 102 also can be characterized with reference to the collision kinematics of the precursor ions 102 with the buffer gas. For example, such collisions may be characterized by an average collision energy, which in turn can represent a degree to which the kinetic energy of each precursor ion 102 is increased as a result of such collisions. As examples, the fragmentation of the precursor ions 102 can be performed such that an average collision energy between the precursor ions 102 and the buffer gas as disclosed herein.

In some examples, the collision energy also can be expressed as a normalized collision energy as disclosed herein. As examples, the fragmentation of the precursor ions 102 can be performed such that an average normalized collision energy between the precursor ions 102 and the buffer gas as disclosed herein.

The collision energy (or normalized collision energy) characterizing the interaction between the precursor ions 102 and the buffer gas can be at least partially determined and/or affected by any of a variety of parameters. As an example, such collision energy may be related to the trapping parameter q, which in turn can be determined and/or varied through variation of any of the parameters discussed above.

Additionally or alternatively, such collision energy may be related to one or more properties of the applied auxiliary electric field, such as the auxiliary voltage amplitude V_{d} and/or the auxiliary field frequency ω_{d}.

Additionally or alternatively, such collision energy can be related to one or more properties of the buffer gas. In particular, in some examples, the buffer gas includes and/or is a light noble gas such as helium gas, which may impart relatively little kinetic energy to the precursor ions 102 in each collision. Alternatively, in other examples, the buffer gas can include and/or be a heavier gas such as nitrogen, argon, and/or xenon, which may impart a relatively larger amount of kinetic energy to the precursor ions in each collision.

Moreover, a pressure of the buffer gas also can affect the prevalence of undesirable fragmentation products in the product ions 206. For example, the collision energy may be related to the amplitude of the micromotion oscillation exhibited by the precursor ions 102 as a result of the applied auxiliary electric field. Increasing the pressure of the buffer gas can inhibit the precursor ions 102 from oscillating with such an amplitude without undergoing buffering collisions with the buffer gas. Accordingly, increasing the pressure of the buffer gas can reduce the likelihood that each precursor ion 102 reaches the micromotion amplitude associated with the applied auxiliary electric field, thus reducing the center-of-mass kinetic energy associated with each collision and reducing the degree to which such collisions heat the precursor ions 102.

Additionally, after each precursor ion 102 undergoes a heating collision with the buffer gas that increases the kinetic energy of the precursor ion, the precursor ion generally must maintain an elevated kinetic energy subsequent to collision for a finite period of time (e.g., on the order of characteristic time) before bond cleavage occurs. In some cases, however, collisions between the precursor ions 102 and the buffer gas can be cooling collisions that remove kinetic energy from the precursor ions 102. The prevalence of such cooling collisions may be directly related to the pressure of the buffer gas. Accordingly, an increase in the pressure of the buffer gas can increase the likelihood that such cooling collisions occur prior to dissociation, reducing a time interval between collisions in which dissociation can occur and resulting in “softer” fragmentation characterized by fewer species of the produced ions 206.

As a result, effective and controlled fragmentation of the precursor ions 102 also may be facilitated by balancing additional parameters, such as to produce collisions between the precursor ions 102 and the buffer gas with a desired collision energy while enabling the precursor ions 102 to remain at the elevated kinetic energy level for sufficient time to allow for dissociation. For example, whereas decreasing the target trapping parameter (e.g., from qₜ=0.25 to qₜ=0.15) can beneficially inhibit the generation of higher-generation fragmentation products when using a given buffer gas (e.g., helium) at a given pressure (e.g., 6 mTorr), a similar mass spectrum of the product ions 206 also may be obtained with a trapping parameter of qₜ=0.25 by raising the pressure of the buffer gas by a suitable amount (e.g., to 12 mTorr).

In some examples, the pressure of the buffer gas also may be related to the amplitude of the micromotion of the precursor ions 102, which in turn can be related to the auxiliary voltage amplitude V_{d}. For example, because lower pressures of the buffer gas can be related to fewer cooling collisions between the precursor ions 102 and the buffer gas, such lower pressures also can enable the use of correspondingly lowered values of the auxiliary voltage amplitude V_{d}.

In some aspects, the selection of the buffer gas can be related to the optimization of the target trapping parameter qₜ characterizing the precursor ions 102. For example, because the kinetic energy imparted to the precursor ions 102 upon collision is related to the mass of the buffer gas, the use of a buffer gas with a relatively high mass can enable moving the precursor ions 102 with relatively small amplitudes (e.g., of secular motion and/or micromotion) to achieve fragmentation. Therefore, the use of a relatively massive buffer gas (e.g., nitrogen) can enable effective fragmentation to occur at relatively low values of qₜ. For example, using a buffer gas in the form of nitrogen with a target trapping parameter of qₜ=0.1 can result in a similar mass spectrum of produced ions 206 as using a buffer gas in the form of helium with a target trapping parameter of qₜ=0.15.

### B. Oligonucleotide Chemistry and Sequencing Considerations

In some aspects of the present disclosure, the method may comprise performing de novo sequencing of oligonucleotides via tandem mass spectrometry. In the absence of a reference sequence to which the product ions can be compared, de novo sequencing relies upon generating mass spectrometry spectra from which the identity and sequence of the product ions can be accurately determined. In the case of large macromolecular complexes such as oligonucleotides, high-energy fragmentation of the precursor ions can produce a wide variety of product ions representing overlapping subsets of the macromolecular structure. This produces mass spectra that are difficult to interpret. Accordingly, de novo sequencing of oligonucleotides can be facilitated by fragmenting the precursor ions in a controlled and/or predictable manner, such as by preferentially cleaving the precursor ions at well-defined labile bonds.

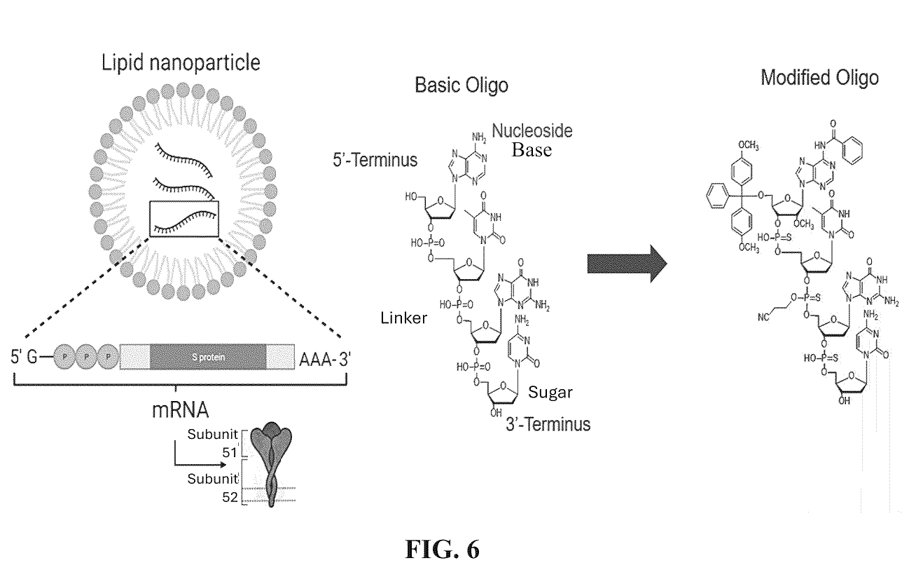

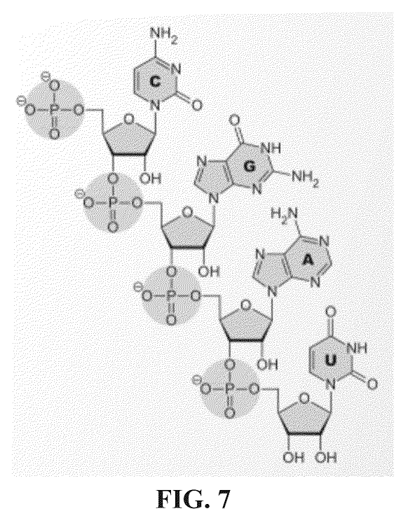

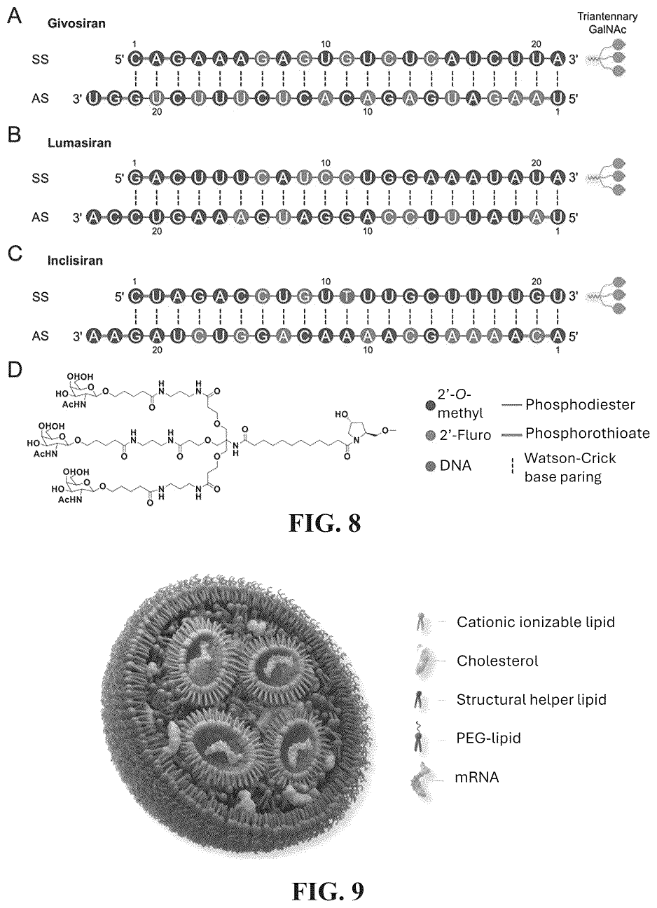

Oligonucleotide fragmentation is highly sensitive to fragmentation conditions and minor changes in how the ions are activated can dramatically impact the quality of the spectra. Over-activation, for example, may generate a plethora of uninformative ions, which complicates interpreting the mass spectra. The difficulty in fragmenting oligonucleotides can in part be attributed to the physiochemical properties of biomolecules, which defines how they behave in a mass spectrometer. Biomolecules comprising oligonucleotides can come in a variety of different compositions. For example, as depicted by FIG. 6, lipid nanoparticles may comprise an oligonucleotide such as mRNA molecule. Such oligonucleotides are first separated from the lipid outer layer before oligonucleotides suitable for mass analysis can be prepared. Oligonucleotide sequencing also must be suitable for sequencing naturally occurring nucleic acids, but also must be suitable for use with modified oligonucleotides. As yet another example, it would be advantageous to sequence double stranded nucleic acids, such as double stranded RNA, e.g. as siRNA, which shuts down genes by degrading mRNA. Certain commercially available RNA therapeutics are illustrated in FIG. 8, namely Givosiran, Lumasiran, and Inclisiran. These nucleic acid therapeutics are approximately 20mers that are substantially modified. For example, as shown by FIG. 8, siRNA can be modified with a 2′-O-methyl, a 2′-Flouro, or may have a phosphodiester or phosphorothioate backbone.

The anatomy of a lipid nanoparticle as illustrated by FIG. 9 comprising an oligonucleotide also presents unique challenges for de novo sequencing of the oligonucleotide. Specifically, the size, heterogeneity, ionization efficiency, and cationic lipid content of the lipid nanoparticle, as well as the resolution/mass accuracy, ion activation method, ion-ion reactions, and component separations are all important mass spectrometry considerations.

In some aspects, the oligonucleotide may comprise ribonucleotide monomers, deoxyribonucleotide monomers, or a combination thereof. A person of ordinary skill in the art will appreciate that oligonucleotides may be of different sizes/lengths, as determined by the number of nucleotides used to form a nucleic acid polymer, which may vary from at least 2 nucleotides to hundreds, if not thousands, of nucleotides. But for purposes of efficient and accurate sequencing, the present disclosure is primarily concerned with oligonucleotides comprising from 2 nucleotides to at least 200 nucleotides, more typically from at least 20 nucleotides to 150 nucleotides, such as 20 nucleotides to 100 nucleotides.

Oligonucleotides include a 5′ end and a 3′ end. The 5′ end of an oligonucleotide may be a hydroxyl, a hydrophobic moiety, a 5′ cap, a phosphate, a diphosphate, a triphosphate, a phosphorothioate, a diphosphorothioate, a triphosphorothioate, a phosphorodithioate, a diphosphrodithioate, a triphosphorodithioate, a phosphonate, a phosphoramidate, or a neutral organic polymer. The 3′ end of an oligonucleotide can be a hydroxyl, a hydrophobic moiety, a phosphate, a diphosphate, a triphosphate, a phosphorothioate, a diphosphorothioate, atriphosphorothioate, a phosphorodithioate, a disphorodithioate, a triphosphorodithioate, a phosphonate, a phosphoramidate, or a neutral organic polymer. An oligonucleotide having a 5′-hydroxyl or 5′-phosphate can have an unmodified 5′ terminus. An oligonucleotide having a 5′ terminus other than 5′-hydroxyl or 5′-phosphate can have a modified 5′ terminus. An oligonucleotide having a 3′-hydroxyl or 3′-phosphate can have an unmodified 3′ terminus. An oligonucleotide having a 3′ terminus other than 3′-hydroxyl or 3′-phosphate can have a modified 3′ terminus. Oligonucleotides can be single or double stranded. Double-stranded oligonucleotide molecules may include one or more single-stranded segments, such as overhangs.

An internucleoside linkage may be an unmodified internucleoside linkage or a modified internucleoside linkage. An unmodified internucleoside linkage is a phosphate (—O—P(O)(OH)—O—) internucleoside linkage (phosphate phosphodiester). A modified internucleoside linkage is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino, thiodiester, thionocarbamate, siloxane, and N,N′-dimethylhydrazine. Phosphorothioate linkages are phosphodiester linkages and phosphotriester linkages in which one of the non-bridging oxygen atoms is replaced with a sulfur atom.

In some aspects, an internucleoside linkage is a group represented by Formula I

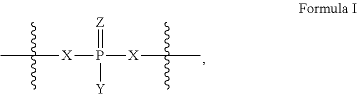

where Z is O, S, or Se; Y is-X-L-R¹; each X is independently —O—, —S—, —N(-L-R¹)—, or L; each L is independently a covalent bond or a linker such as a linker consisting of 1, 2, 3, 4, 5, 6, 7, 8, 9, or 10 monomers independently selected from the group consisting of a substituted C₁₋₁₂ alkylene, a substituted C₂₋₁₂ heteroalkylene, a substituted C₆₋₁₀ arylene, a substituted C₃₋₈ cycloalkylene, a substituted C₁₋₉ heteroarylene, a substituted C₁₋₉ heterocyclylene, —O—, —S—S—, and —NR^{N}—, where each R^{N} is independently H or substituted C₁₋₁₂ alkyl); each R¹ is independently hydrogen, —S—S—R², —O—CO—R², —S—CO—R², substituted C₁₋₉ heterocyclyl, or a hydrophobic moiety; and each R² is independently substituted C₁₋₁₀ alkyl, substituted C₂₋₁₀ heteroalkyl, substituted C₆₋₁₀ aryl, substituted C₆₋₁₀ aryl C₁₋₆ alkyl, substituted C₁₋₉ heterocyclyl, or substituted C₁₋₉ heterocyclyl C₁₋₆ alkyl. When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either L is a linker or R¹ is not a hydrogen, the internucleoside group is known as a phosphotriester. When Z is sulfur, all X groups are —O—, and either L is a linker or R¹ is not a hydrogen, the internucleoside group is known as a phosphorothioate triester.

The nucleobase is a nitrogen-containing heterocyclic ring found at the 1′ position of the ribofuranose/2′-deoxyribofuranose of a nucleoside. Nucleobases may be unmodified or modified. As used herein, “unmodified” or “natural” nucleobases include the purine bases adenine and guanine, and the pyrimidine bases thymine, cytosine, and uracil. Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, for example, 5-methylcytosine, 5-hydroxymethyl cytosine, xanthine, hypoxanthine, 2-aminoadenine, 6-alkyl (for example 6-methyl) adenine and guanine, 2-alkyl (for example, 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e.g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone, and the like.

The nucleoside may comprise a sugar-nucleobase compound or modified or unmodified 2′-deoxyribofuranose-nucleobase compounds. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. A nucleobase modification replaces an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy) ethoxy moiety. Alternatively, a 2′-substitution may be a 2′-(ara) substitution corresponding to Formula II

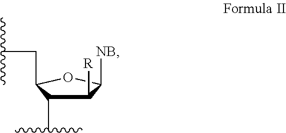

where NB is a nucleobase, and R is a 2′-(ara) substituent (e.g., fluoro). A locking modification is an incorporation of a bridge between 4′-carbon atom and 2′-carbon atom of ribofuranose. Nucleosides having a locking modification include bridged nucleic acids, e.g., locked nucleic acids (LNA), ethylene-bridged nucleic acids (ENA), and cEt nucleic acids. The bridged nucleic acids are typically used as affinity enhancing nucleosides.

Nucleotides comprise a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently alkyl, or both R² groups, together with the nitrogen atom to which they are attached, combine to form a substituted heterocyclyl.

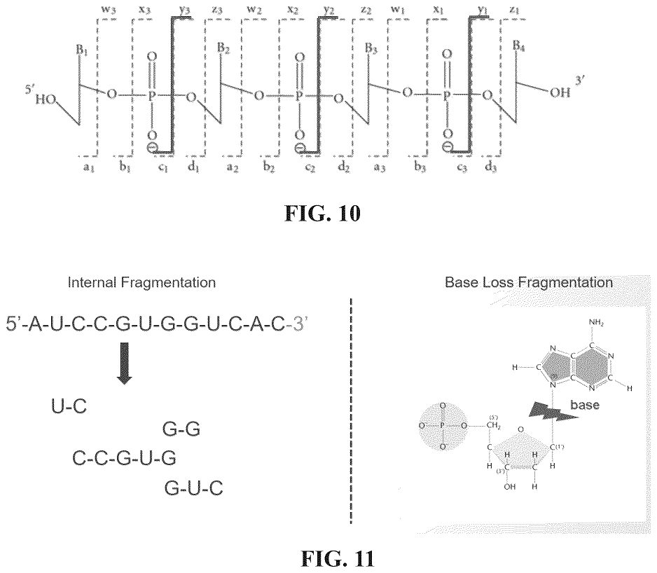

In some aspects, the product ions disclosed herein follow the nomenclature scheme depicted in FIG. 10. FIG. 10 illustrates an oligonucleotide precursor ion comprising nucleobases B₁-B₄. Potential bond cleavage points along phosphodiester bond are indicated by lower case a, b, c, and d for fragments from the 5′-OH group; w, x, y, and z are designated for fragments from the 3′-OH group; and the numerical transcripts refer to the number of nucleobases from the respective termini. High quality spectra are generated from a high abundance of c ion and y ion fragments because they are sequence informative fragments. One aspect of the present invention is that conditions suitable for providing sequence useful information result in “soft fragmentation.” Base loss fragmentation from precursor ions may not be sequence informative but can indicate the quality of spectra (i.e., soft fragmentation). On the other hand, complicated mass spectra resulting from harsh fragmentation conditions are more likely is generated from internal fragment ions, fragment base losses, and fragment ion water losses. Moreover, internal fragment ions can give a signal identical to sequence informative c and y ions of incorrect sequences, which can result in incorrect sequence analysis.

In particular aspects of the present disclosure, precursor ions are dissociated using collision induced dissociation to form product ions comprising fragments of the precursor ions, wherein the precursor ions can be oligonucleotides. Oligonucleotide fragments may be formed by a single cleavage along the phosphodiester backbone in the 5′ and 3′ directions. To provide direct sequence information, chemical bonds can be fragmented as illustrated by Scheme I.

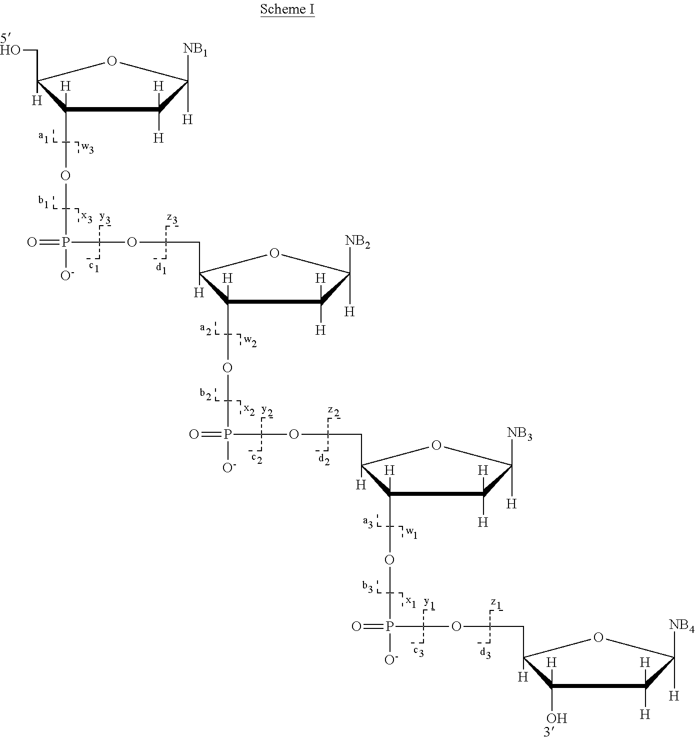

As illustrated by Scheme I, a DNA oligonucleotide comprising four nucleobases NB₁-NB₄ is fragmented along the phosphodiester bond. The cleavages are indicated by ‘a,’ ‘b,’ ‘c’, and ‘d’ for fragments comprising the 5′-OH group, and ‘w’, ‘x’, ‘y’, and ‘z,’ for fragments comprising the 3′-OH group. Again, the numerical subscripts indicate the number of bases from the respective termini. Current methods typically use harsh fragmentation conditions, cleaving the oligonucleotide at multiple locations, result in internal fragmentation and base loss fragment, which affect the sensitivity and specificity resulting in a complex and uninformative mass spectra data, particularly as the length of the oligonucleotide increases.

In contrast, embodiments of the disclosed method provide direct sequence information. For example, an oligonucleotide is preferably fragmented along the phosphodiester backbone to form c-ions and y-ions, such as a c₁ ion fragment

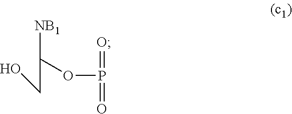

and a y₃ ion fragment

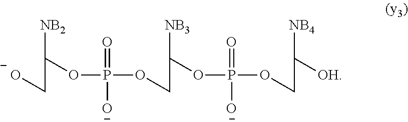

In some aspects, precursor ions comprising RNA oligonucleotides are dissociated using collision induced dissociation to form product ions comprising fragments of the RNA oligonucleotides as illustrated by Scheme II.

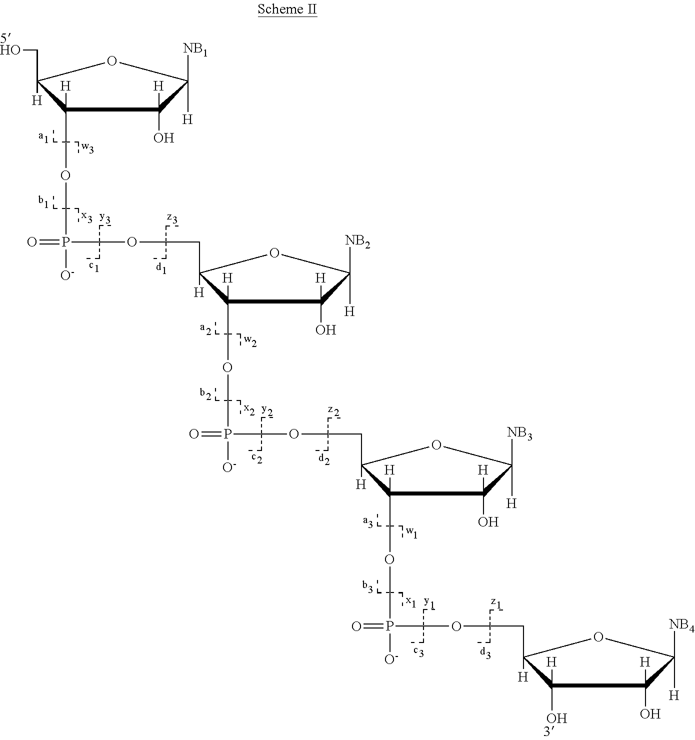

The RNA oligonucleotide is cleaved along the phosphodiester bond, which are indicated by ‘a,’ ‘b,’ ‘c’, and ‘d’ for fragments from the 5′-OH group, and ‘w’, ‘x’, ‘y’, and ‘z,’ for fragments from the 3′-OH group. Again, the numerical subscripts indicate the number of bases from the respective termini. Embodiments of the disclosed method produce ion fragments that provide direct sequence information. For example, an oligonucleotide is preferably fragmented along the phosphodiester backbone to form c-ions and y ions, such as a c₁ on fragment

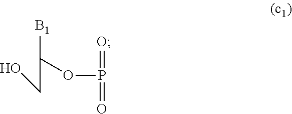

and a y₃ ion fragment

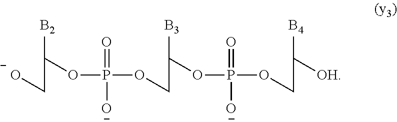

The disclosed method can also be used to sequence modified oligonucleotides, such as an antisense oligonucleotide comprising a phosphorothioate backbone. For example, Scheme III depicts an oligonucleotide having a phosphorothioate backbone comprising four bases NB₁-NB₄.

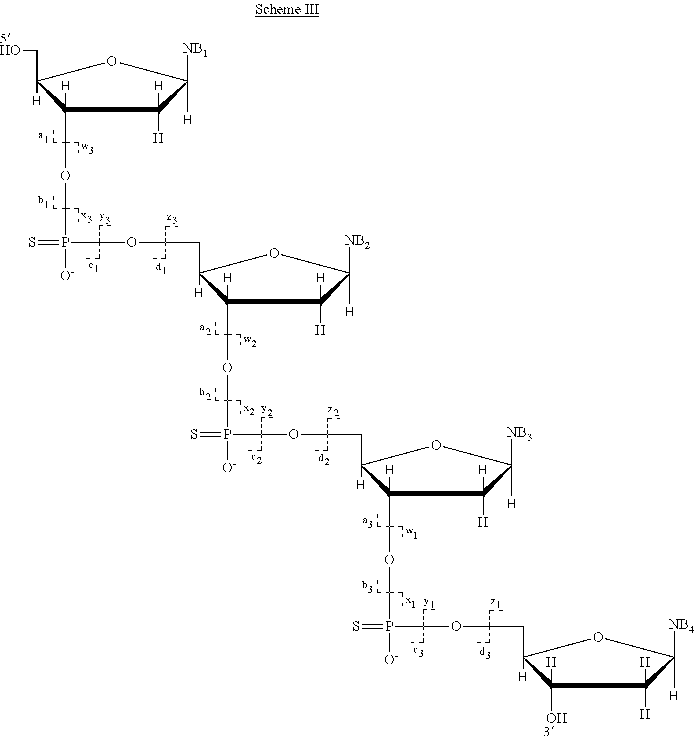

The oligonucleotide comprising a phosphorothioate is preferably fragmented along the phosphorothioate backbone to form c-ions and y ions, such as a c₁ on fragment

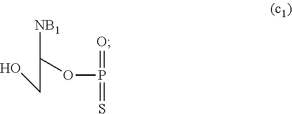

and a y₃ ion fragment

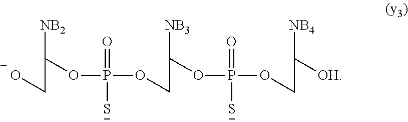

As yet another example, the oligonucleotide can be a modified RNA oligonucleotide, such as an oligonucleotide comprising a 2′O-methyl modification. Scheme IV illustrates the modified RNA oligonucleotide comprising four bases.

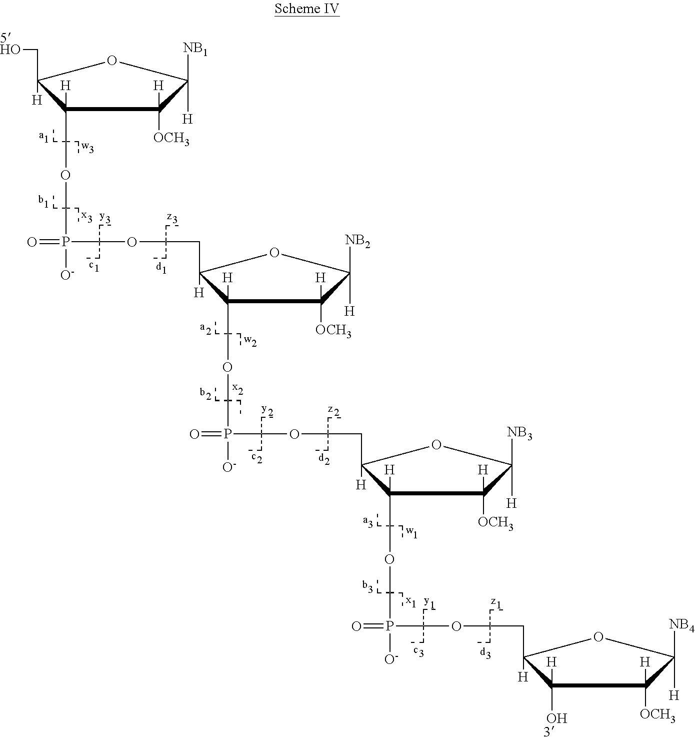

The modified RNA oligonucleotide comprising a 2′O-methyl modification is preferably fragmented along the phosphodiester backbone to form c-ions and y ions, such as a c₁ on fragment

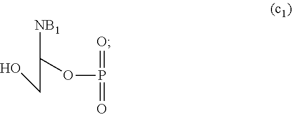

and a y₃ ion fragment

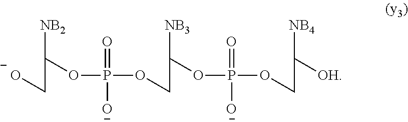

In particular aspects disclosed herein, the modified oligonucleotide may comprise a modified nucleobase. For example, the RNA oligonucleotide may comprise a modified nucleobase. Scheme V depicts an RNA oligonucleotide comprising four nucleobases.

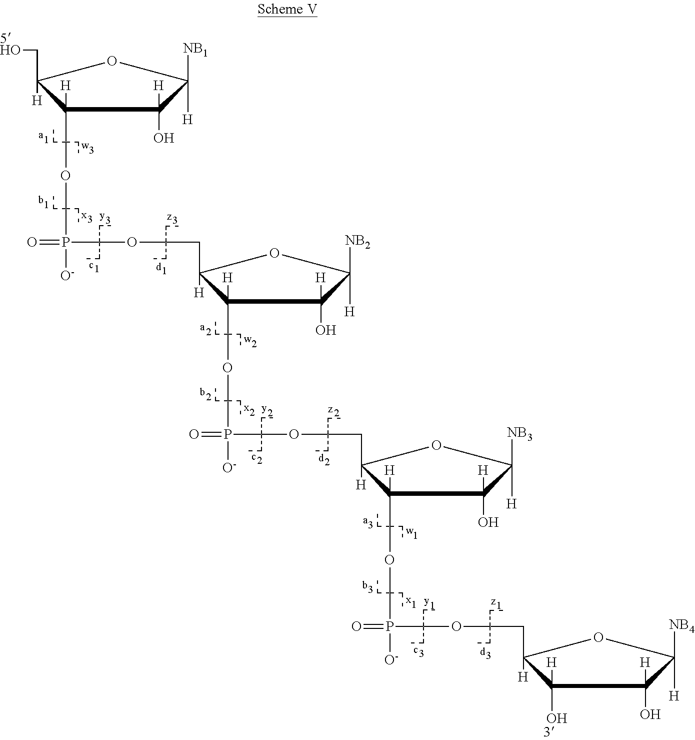

B₁-B₄ may comprise a modified nucleobase, such as a 5-ribosyluracil (pseudouracil) and the modified RNA oligonucleotide is preferably fragmented along the phosphodiester backbone to form c-ions and y ions, such as a c₁ on fragment

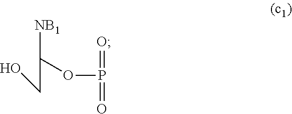

and a y₃ ion fragment. In another exemplary aspect, the modified nucleobase is 5-methylcytosine.

In particular aspects disclosed herein, the nuclease cane be RNase T1 (Thermo Fisher), which cleaves ssRNA at 3′ end of the G residue; MasF (Takara), which cleaves ssRNA at 5′ end of ACA and does not cleave dsRNA, dsDNA, and ssDNA; and the like. In some aspects, beads comprising an enzyme can be used to avoid nuclease interference with subsequent LC-MS analysis and eliminate enzyme contamination in MS-based RNA mapping.

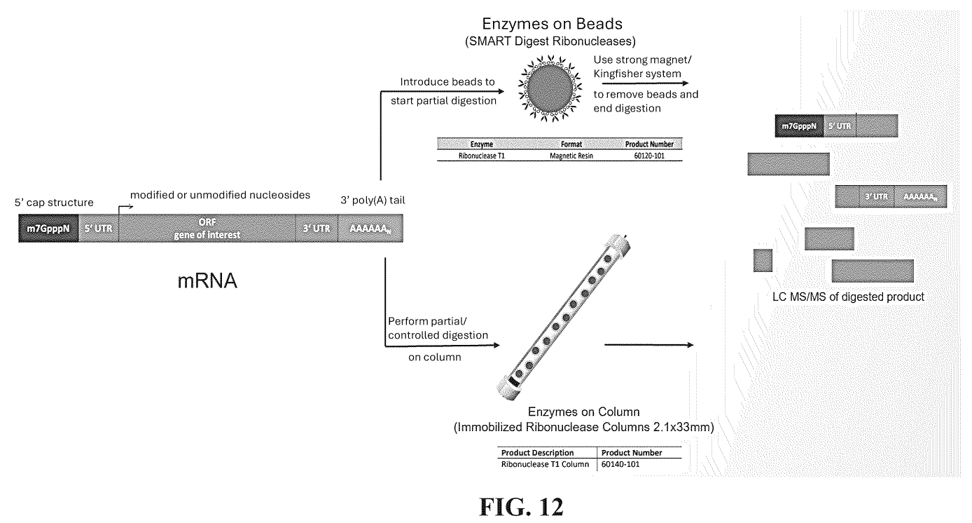

FIG. 12 is a schematic illustrating one aspect of disclosed method embodiments comprising partial enzymatic digest, followed by LC MS/MS of digested products. A person of ordinary skill in the art will understand that any of a number of enzymatic digests might be utilized, such as by using SMART digest ribonucleases. The digest enzymes may be immobilized on beads or in a column. FIG. 12 illustrates using this process for digesting and sequencing mRNA having a 5′cap structure, a 5′UTR with Kozak translation initiation sequence, main sequence/open reading frame (ORF), 3′ UTR with globin sequence, and a 3′ poly(A) tail.

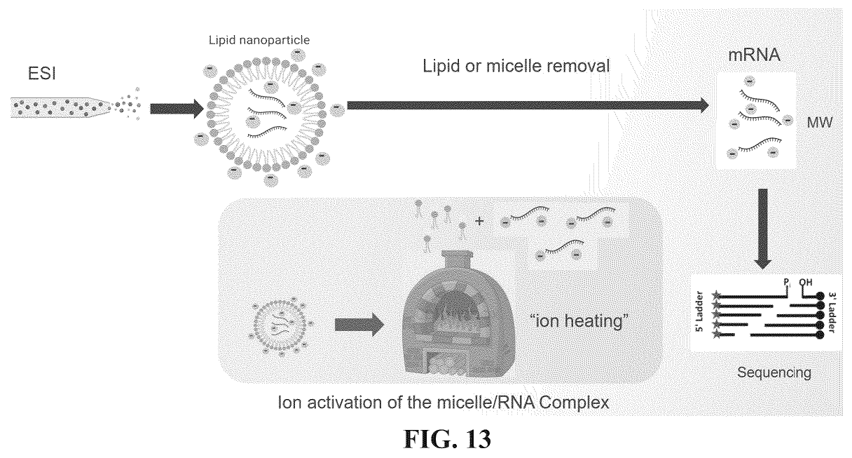

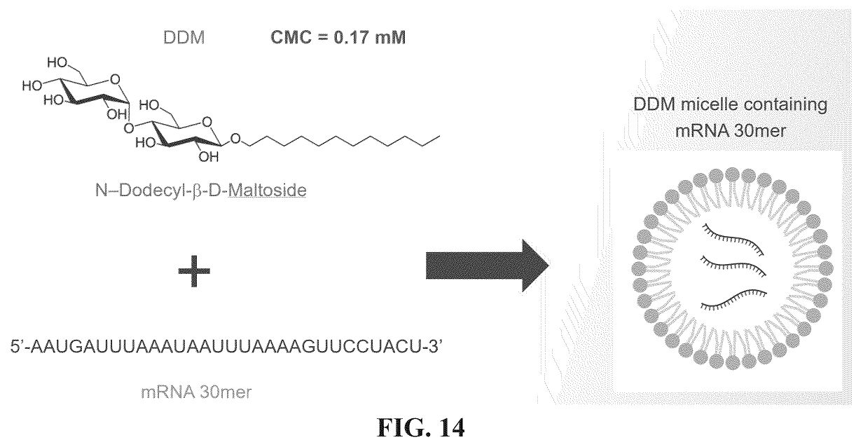

In some aspects, the method may comprise direct sequencing of an oligonucleotide in a lipid nanoparticle. For example, as shown in FIG. 13, a lipid nanoparticle comprising mRNA is negatively charged via ESI. This is followed by ion activation of the micelle/RNA complex by ion heating, which produces fragmented mRNA ions, which are sequenced with low q CID.

### IV. Examples

The following examples are provided to illustrate certain specific features of disclosed embodiments. A person of ordinary skill in the art will appreciate that the scope of the invention is not limited to the particular features of these examples.

Materials: Oligonucleotide samples used in the following examples were purchased and used without further purification from IDT (Integrated DNA Technologies, https://www.idtdna.com). Samples received directly from IDT were desalted using standard procedures. Sample synthesis for smaller oligonucleotides (60mers and below) was conducted via solid phase synthesis methods. Larger oligonucleotides were synthesized using proprietary methodologies. This includes single stranded DNA, single stranded RNA, double stranded RNA, and any type of modified RNA or DNA. The mRNA from eGFP used in the initial digestion experiments was obtained from TriLink Biotechnologies at a concentration of 1 mg/ml. For oligonucleotides up to 60mers, 100 nmol per oligonucleotide was used. For larger oligonucleotides, 20 nmol of material was obtained for experimental use. Stock solutions of oligonucleotides (single stranded) were prepared in water at a concentration of 10 mM.

Dilutions for direct infusion experiments (static spray) were in the 5 mM range with 50 mM ammonium acetate. Samples run via liquid chromatography tandem mass spectrometry were analyzed with 10 μmol injected on column from a purely aqueous solution at a concentration of 10 mM.

Hybridization conditions for double stranded mRNA experiments started with a 25 mM stock solution of each complementary mRNA strand in 150 mM ammonium acetate. Two 100 ml aliquots of each stock solution were combined in an Eppendorf tube and were incubated at 95° C. in a heating block and were then cooled overnight. The cooling process was performed at room temperature to limit the kinetics to those favorable for strand hybridization. Direct infusion of the hybridized product was performed by loading 3 to 5 ml into a glass nano-emitter using a 10 ml Hamilton gas-tight syringe.

For mRNA based digestion of eGFP, 20 mg of material was used as a starting point. The enzyme used for digestion was T1 RNAse bound to magnetic beads obtained from Thermo Fisher Scientific. The enzyme cleaves mRNA on the 3′ side of guanosine. Since the amount of enzyme bound to a bead is variable, it was difficult to get an exact enzyme to substrate ratio. For the initial experiment, 2.5 mL beads were added into 20 mL of the 1 ug/mL eGFP substrate. Thermo Fisher brand SMART buffer was used for the digestion which contains about 0.5 M of various salts including at least 50 mM Tris buffer. The reaction was allowed to run for 1 hour at 37° C. while shaking during the incubation period. Beads with the bound RNAse T1 were removed with a magnet and the sample was filtered through a 0.2 micron filter before injection into the LC-MS/MS system (Lumos).

### Example 1

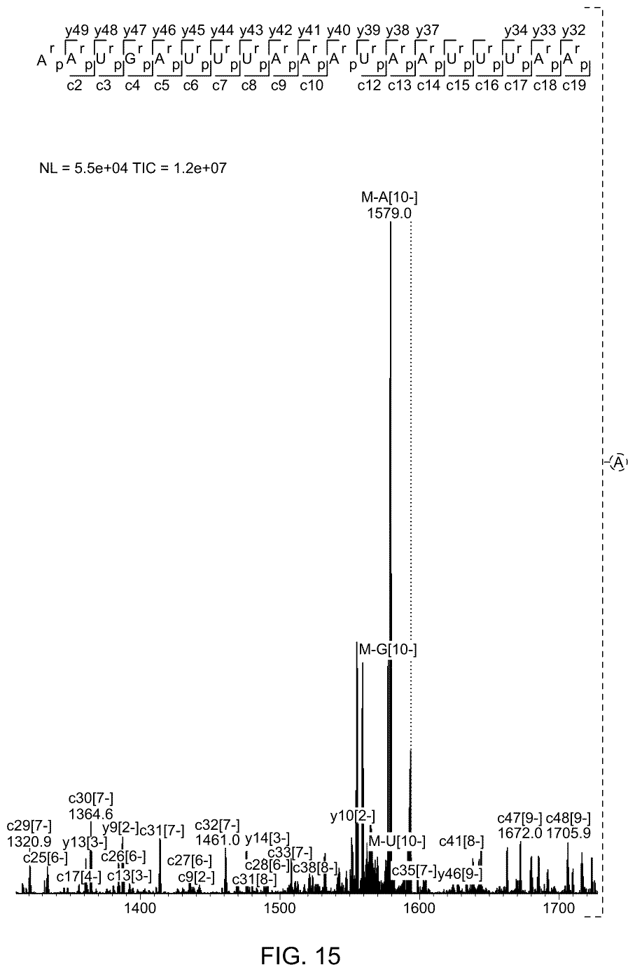

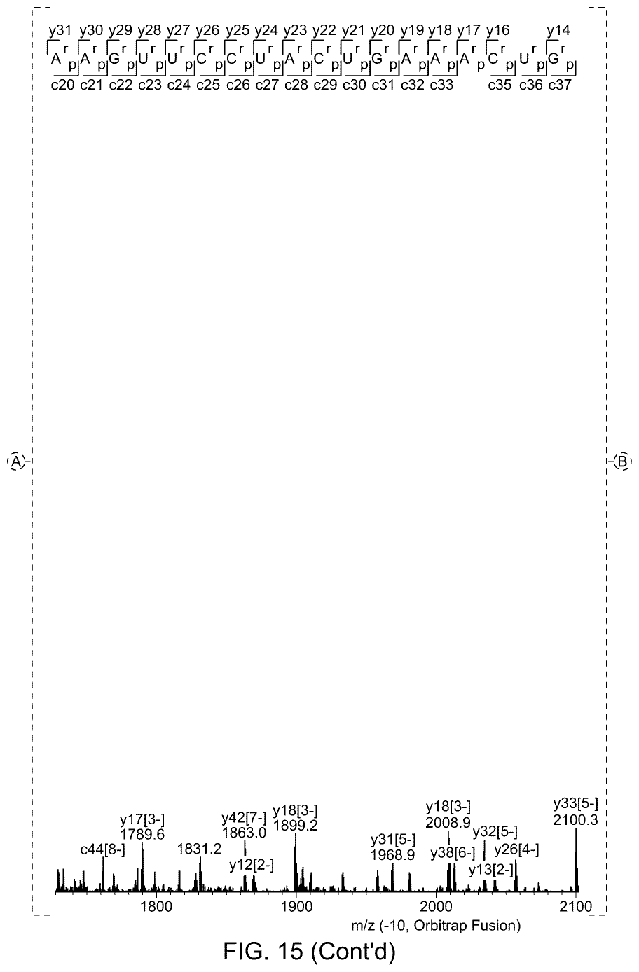

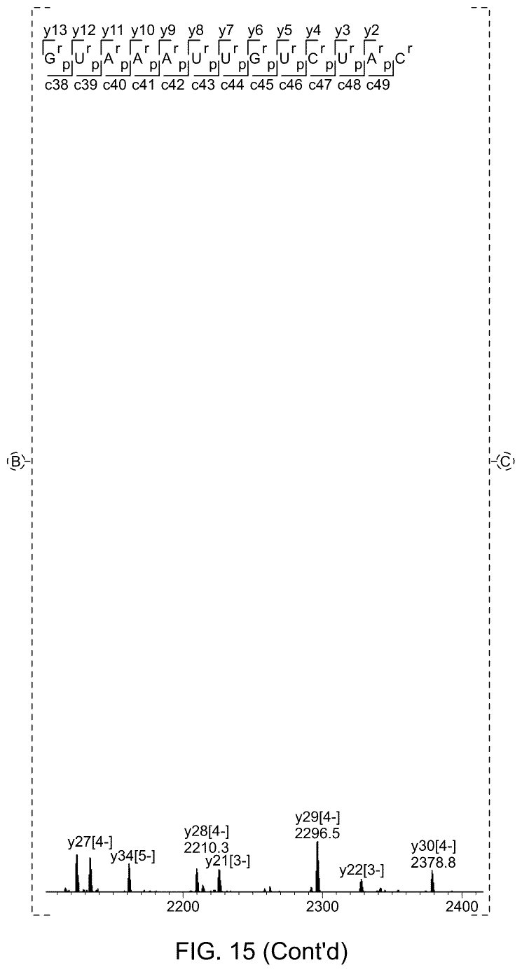

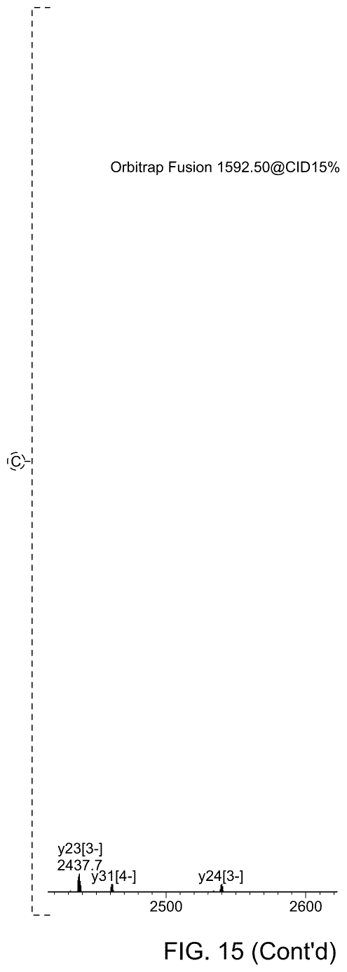

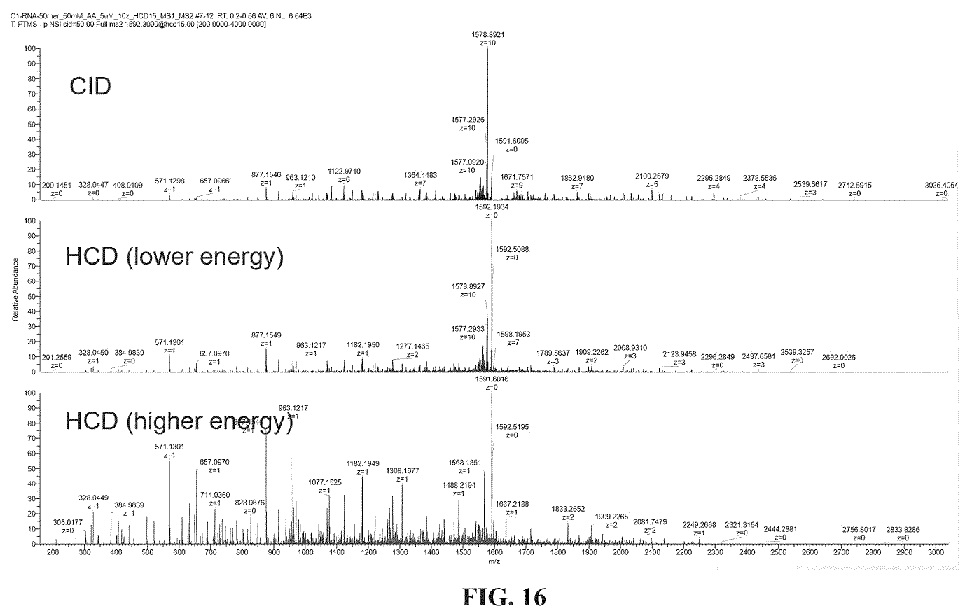

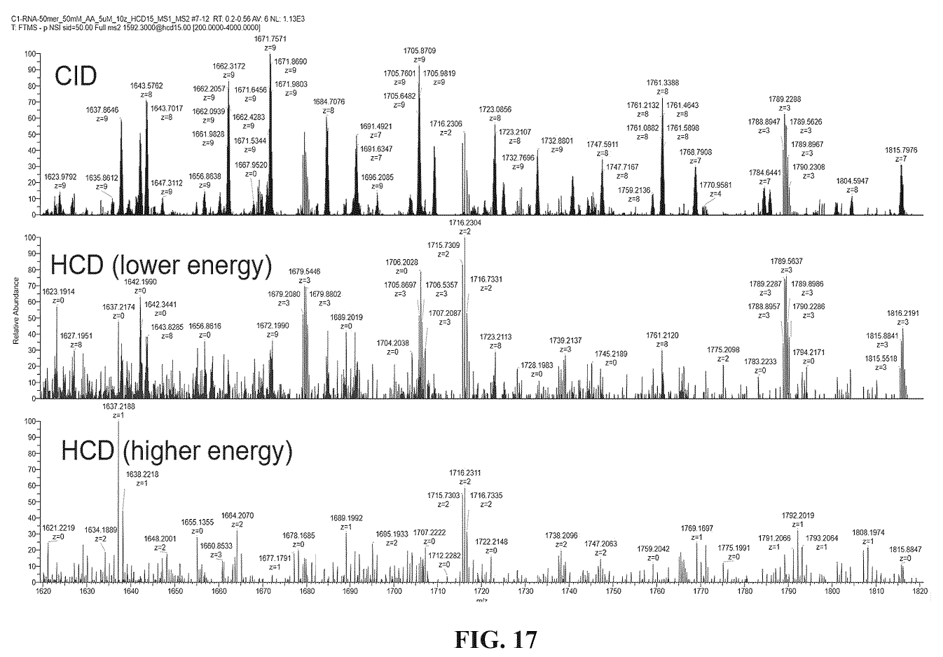

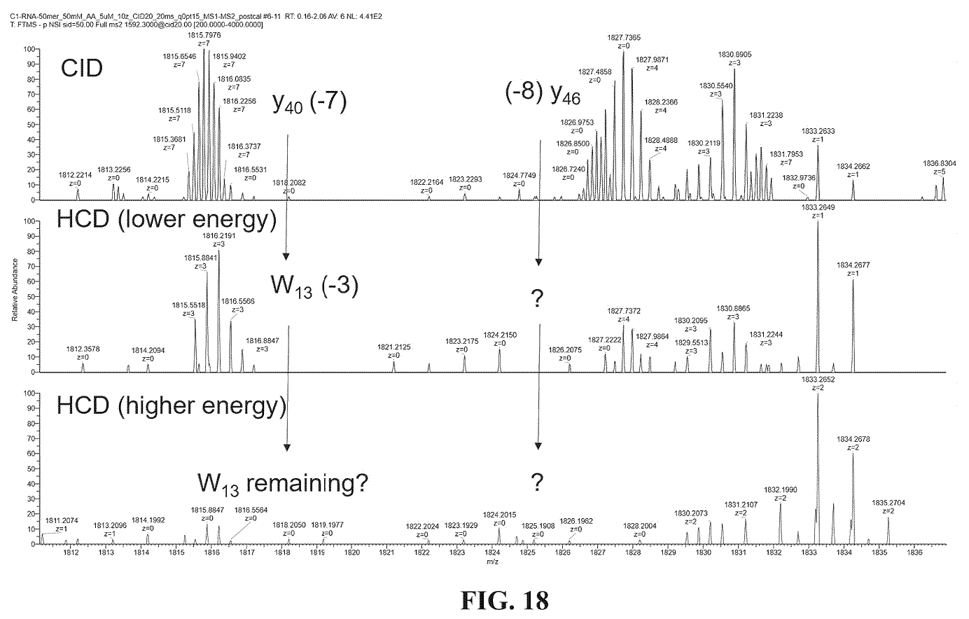

This example concerns obtaining mass spectra of an RNA 50mer having SEQ ID NO: 10, i.e. 5′ [A-A-U-G-A-U-U-U-A-A-A-U-A-A-U-U-U-A-A-A-A-G-U-U-C-C-U-A-C-U-G-A-A-A-C-U-G-G-U-A-A-A-U-U-G-U-C-U-A-C 3′] that compares using low-q CID versus HCD at low energy and high energy. FIG. 15 provides the product ion mass spectrum at a q of 0.15 for the −10 charge state precursor ion of the intact 50mer of RNA. The highest relative abundance peak observed are a few base loss ions from the intact 50mer (peaks labeled in green). The majority of the product ions are comprised of c- and y-type ions indicated in red and blue respectively. The relative intensities of these product ions are approximately equal due to the charge density of the precursor and therefore equal distribution of internal vibrational modes of energy as a result of the CID process. The fine control of the ion velocities described in this application as a result of the q value and other relevant parameters produces primarily complementary c/y complementary ion pairs in the procut ion mass spectrum. FIG. 16 shows the product ion mass spectrum obtained using low q CID compared to the product ion data obtained using lower energy (threshold) HCD, and higher energy HCD, of the RNA 50mer derived from the precursor ion from the −10 charge state. The low q CID mass spectrum produces primarily base loss fragments from the precursor and c/y complementary fragment ions. Shifting to HCD at a threshold (lower) value for inducing a small percentage of dissociation of the −10 charge state precursor ion, a significant portion of the intact precursor is observed. The observed sequence fragments even at this threshold value are significantly greater in number than those observed with low q CID. This is due to the excess energy imparted to the precursor ion as a result of traditional beam type CID experiments. The multiple fragmentation pathways produced as a result of this process complicate the mass spectral interpretation of the product ion spectrum and can even lead to erroneous mass peak assignment. For traditional values of HCD energy (higher) the resulting fragments produced are primarily singly-charged production ions with resulting in very incomplete and sporadic sequence coverage of the intact RNA 50mer. This is indicated by the high intensity singly-charged peaks below the m/z value of the −10 charge state precursor ion. FIG. 17 provides a zoom view from 1620 m/z to 1816 m/z from FIG. 16 comparing the mass spectral results for low q CID (top), lower energy HCD (middle), and the higher energy HCD (lower). The low q CID data provides high signal-to-noise ratio data with high charge states up to −9 for the product ions. This results in large fragment ions and a nearly complete series of complementary c/y ion pairs. For the lower energy CID data, some a few sporadic higher charge states are observed but the majority of the production ions are found at charge states between −3 and −1 in the product ion mass spectrum. A higher density of fragment ions is observed when compared to the low q CID data with majority the result of overfragmentation and the loss of sequence confirmation. Finally at typically observed HCD energies, the majority of fragment ion species observed are between the −2 and −1 charge states with an even higher number of products produced as the result of higher energy collisions. FIG. 18 is a zoom view of FIG. 17 ranging from m/z 1811 to 1837. where low q CID data is shown at the top, lower energy HCD data is shown in the middle, and higher energy HCD data is shown at the bottom of the figure. The low q CID data show high intensity, high signal-to-noise and high charge state product ions for two 5′ fragments of the RNA 50mer the y40 (−7) and the y46 (−8) over this narrow m/z window. Even with lower HCD energies for beam type dissociation (middle), there is a significant loss of sequence information due to excess energy deposited into the precursor ion charge state. This is evident by the disappearance of the y40 (−7) and y46 (−8) charge states. At lower HCD energies there is a w13 (−3) product ion observed, however this is still a substantial loss of sequence information as the result of overfragmentation.

### Example 2

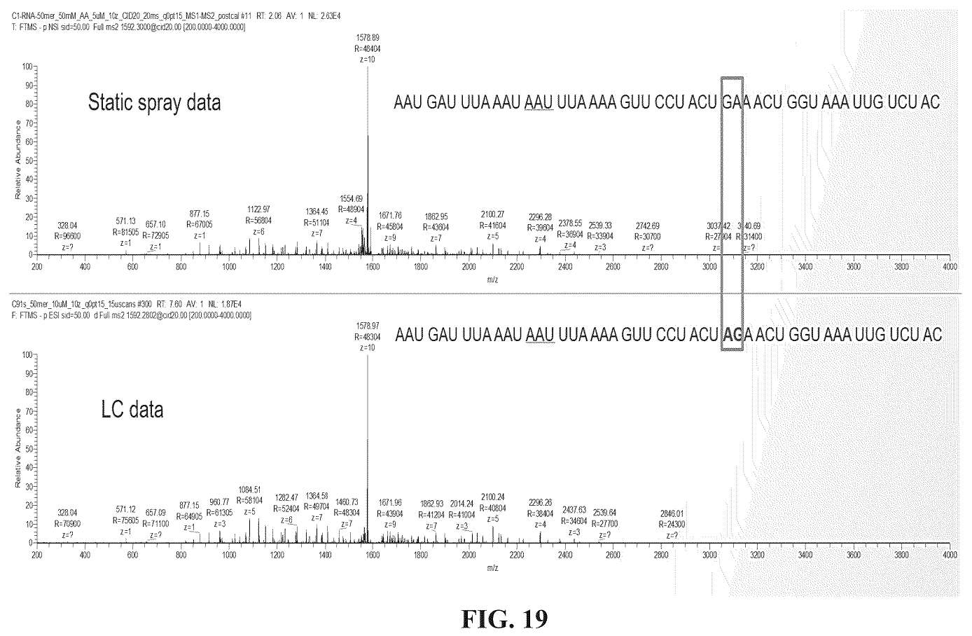

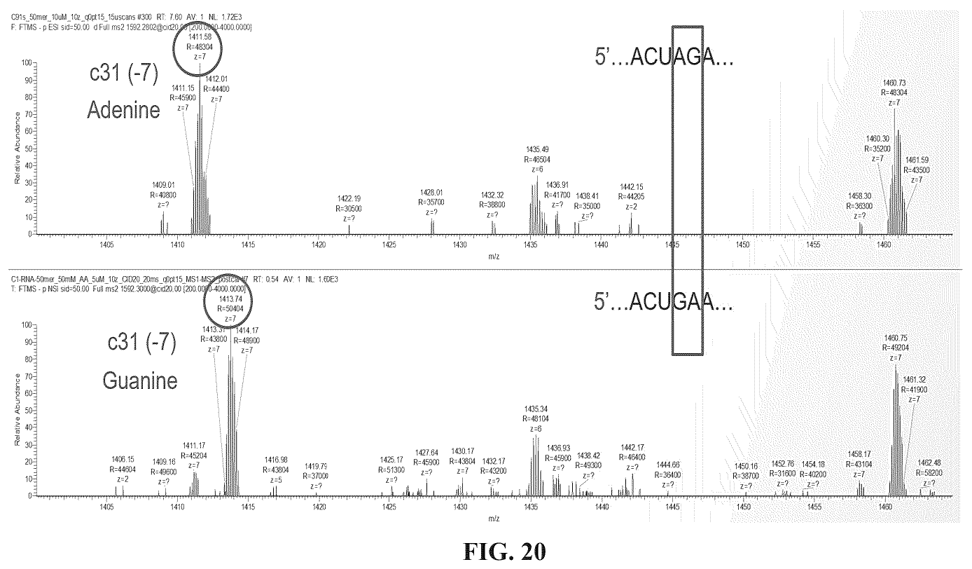

In this example, 50mers with a single base change (A/G swap) were distinguished from one another using the low q CID approach. FIG. 19 provides mass spectrometry static spray data for a 50mer (SEQ ID NO: 9), namely 5′ A-A-U-G-A-U-U-U-A-A-A-U-A-A-U-U-U-A-A-A-A-G-U-U-C-C-U-A-C-U-A-G-A-A-C-U-G-G-U-A-A-A-U-U-G-U-C-U-A-C 3′ and the LC data of 50mer (SEQ ID NO: 10) with a single base swap with the sequence 5′ A-A-U-G-A-U-U-U-A-A-A-U-A-A-U-U-U-A-A-A-A-G-U-U-C-C-U-A-C-U-G-A-A-A-C-U-G-G-U-A-A-A-U-U-G-U-C-U-A-C 3′. The swap of A/G occurs in position 31-32 counting from the 5′ end of the RNA 50mer. The data were acquired one month apart on the same instrument using low q CID using two different sample introduction techniques. Upon initial inspection the observed production spectra appear to be the same. FIG. 20 is a zoom view from FIG. 19 showing the 5′ fragment c31 (−7) product ion region for both the original and A/G swap RNA 50mers. The c31 (−7) product ion at m/z 1413.74 indicates the change in position of these two nucleosides in the 50mer (lower graph).

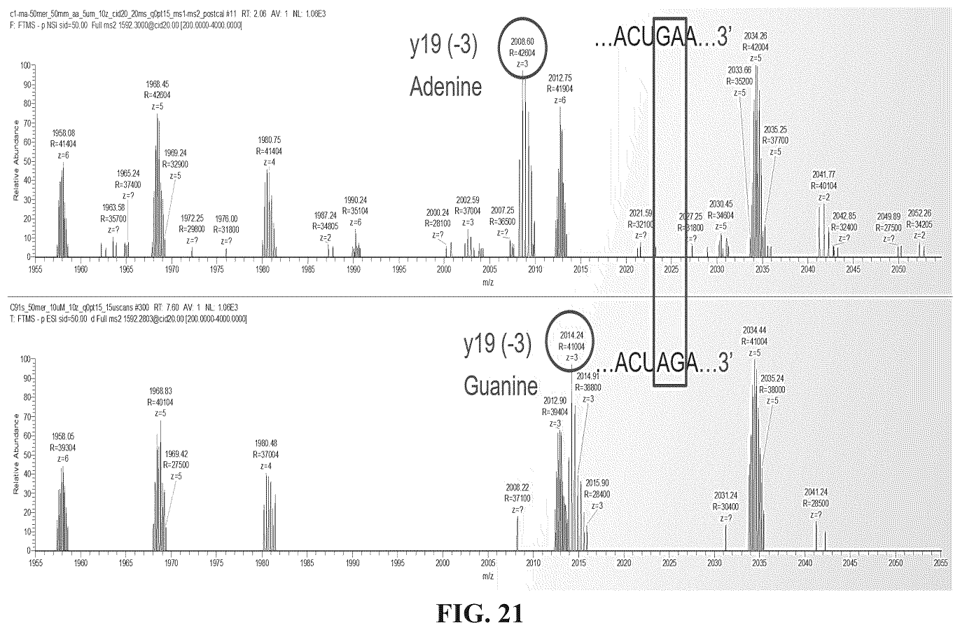

FIG. 21 is a zoom view from FIG. 19 showing the 3′ fragment y19 (−3) product ion region for both the original and A/G swap RNA 50mers. The y19 (−3) production at m/z 2014.24 indicates the change in position of these two nucleosides in the 50mer (lower graph).

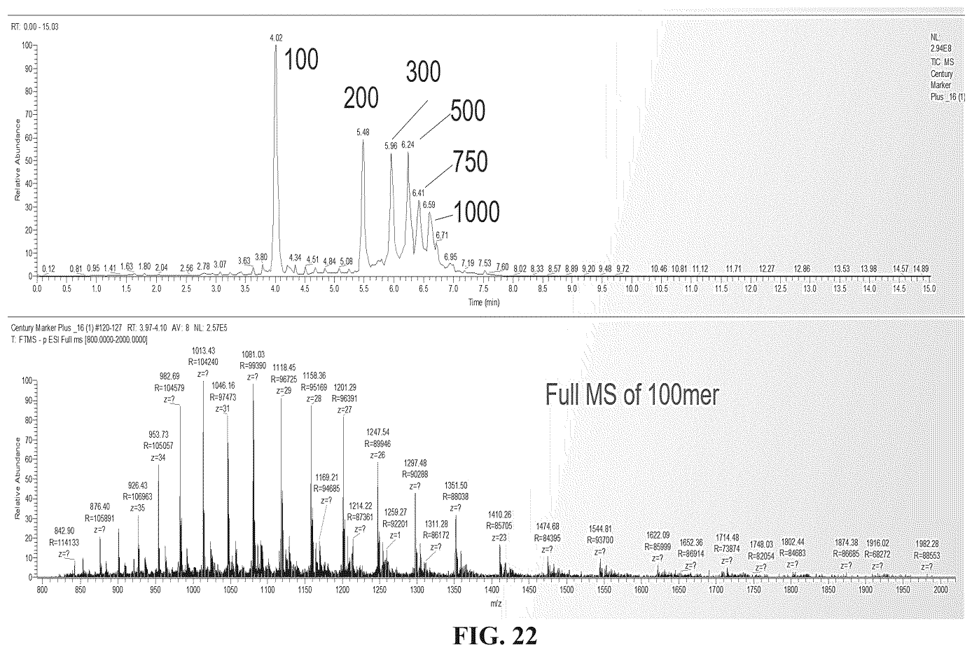

FIG. 22 is an LC-MS chromatogram of a ladder mixture of RNA sequences including a 100, 200, 300, 500, 750 and 1000 mer. Mobile phase conditions employed a hydrogen bond donor/acceptor model utilizing a secondary amine. This resulted in elution times for a 98mer at 4.02 minutes, a 200mer at 5.48 minutes, a 300mer at 5.96 minutes, 750mer at 6.24 minutes, and a 1000mer at 6.59 minutes. FIG. 22 also shows a full scan mass spectrum of a 98mer to demonstrate that intact molecular weights can easily be calculated as well.

### Example 3

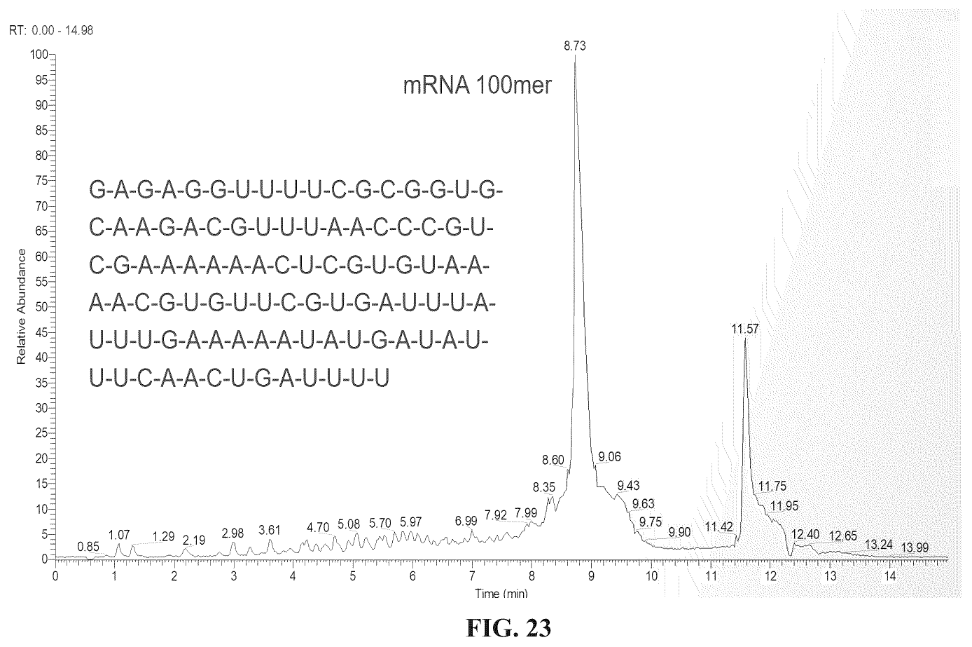

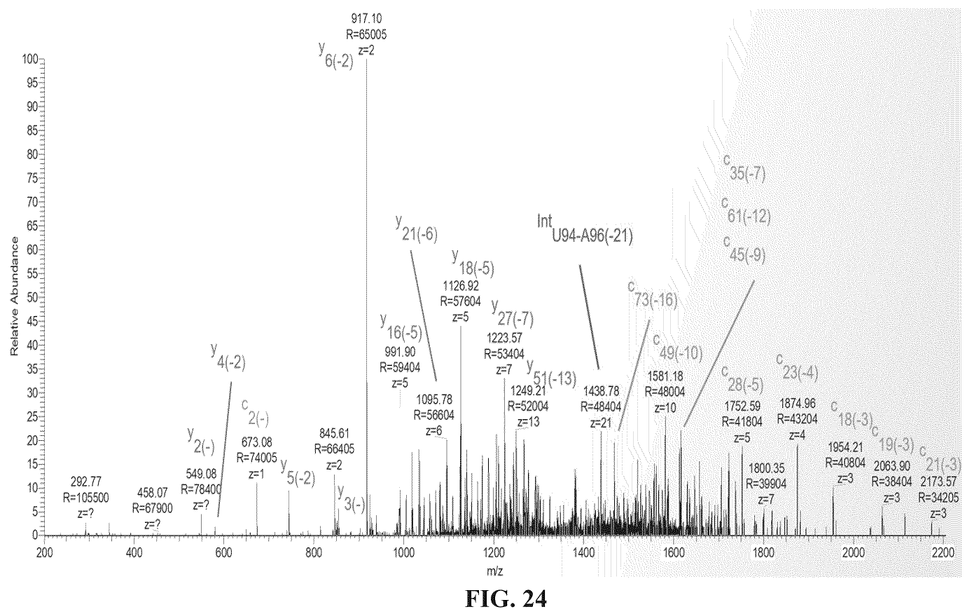

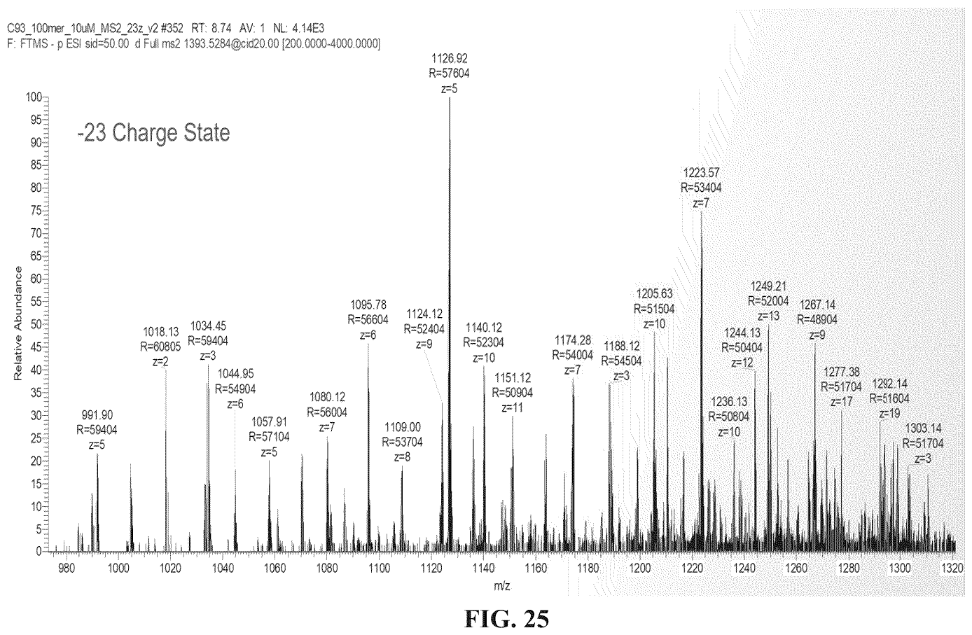

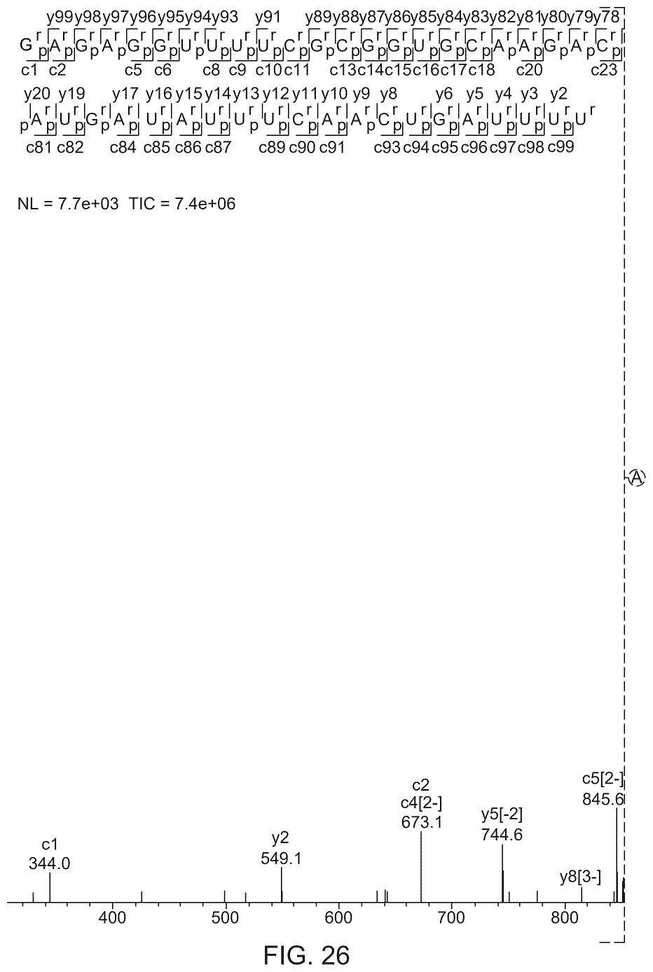

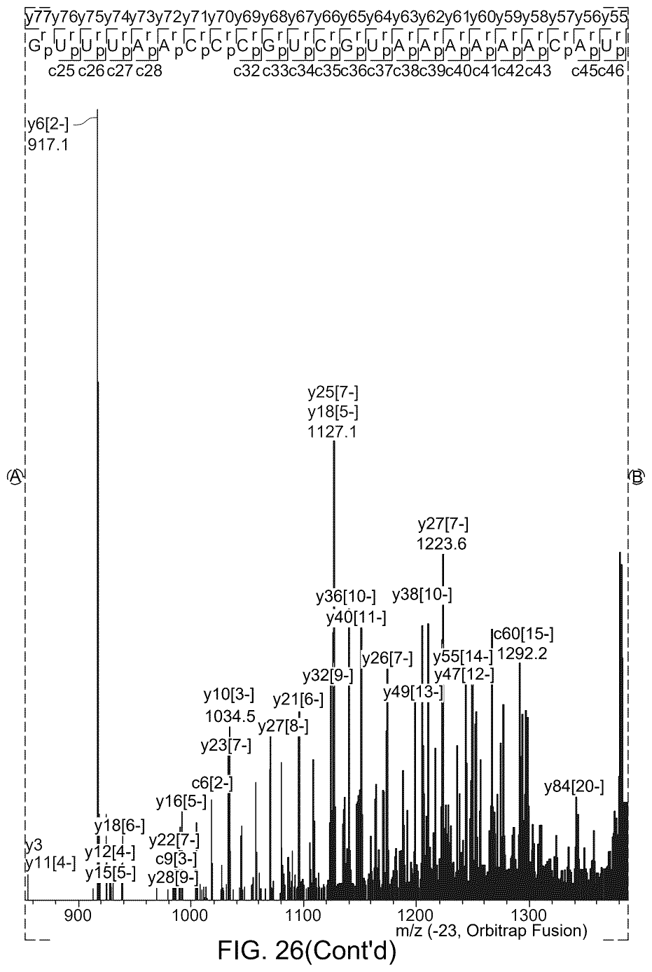

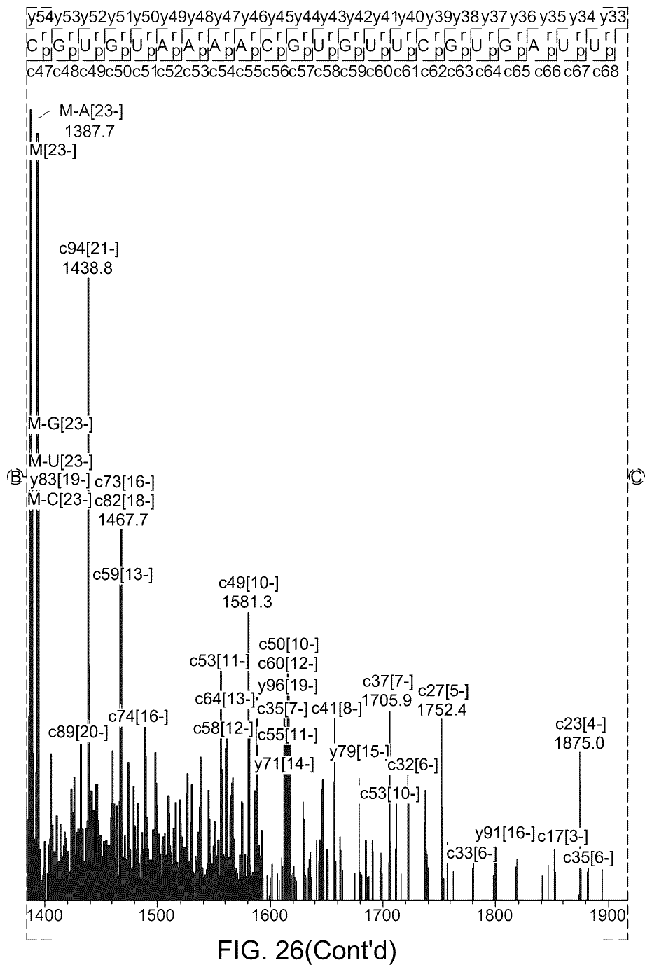

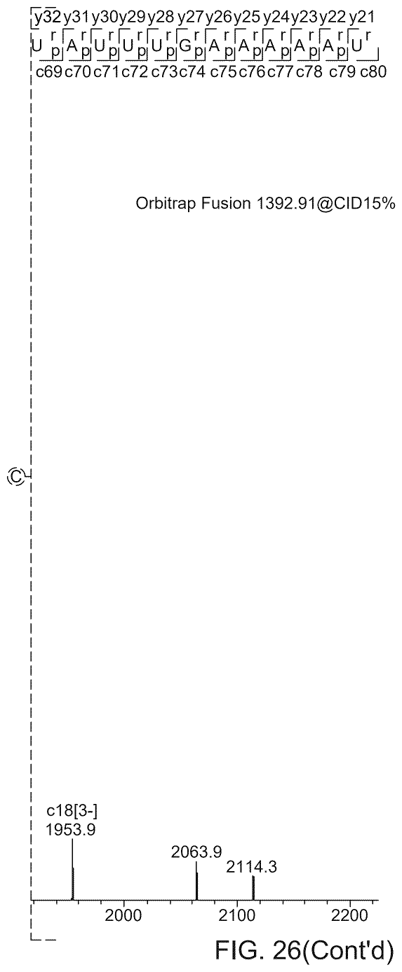

In this example, a 98mer sequence included repeat areas to demonstrate the utility of the low q CID approach in sequencing RNA that contain repeat regions of at least 3 to 6 of the same nucleoside. The 98mer (SEQ ID NO: 11) having sequence 5′ G-A-G-A-G-G-U-U-U-U-C-G-C-G-G-U-G-C-A-A-G-A-C-G-U-U-U-A-A-C-C-C-G-U-C-G-A-A-A-A-A-A-C-U-C-G-U-G-U-A-A-A-A-C-G-U-G-U-U-C-G-U-G-A-U-U-U-A-U-U-U-G-A-A-A-A-A-U-A-U- G-A-U-A-U-U-U-C-A-A-C-U-G-A-U-U-U-U 3′. FIG. 23 shows the chromatographic separation of the 98mer mRNA from contaminants and failed synthesis products at a retention time of 8.73 minutes. FIG. 24 is the product ion mass spectrum from the MS/MS of the −23 charge state of the 98mer at a q of 0.15. Some of the more intense product ions are labeled for convenience. As with previous results obtained using low q CID, the vast majority of the product ions produced are complementary c/y ion series directly related to the sequence of the RNA molecule. FIG. 25 is a zoom view from m/z 970 to 1320 of FIG. 24. This demonstrates the quality of the data obtained for larger RNA sequences using the low q approach. As discussed previously, this is characterized by high charge state larger fragment ions (for example the −19 charge state at m/z 1292). Hence the product ion spectrum is primarily first generation production ions at higher charge states and high s/n ratio. This limited fragmentation maximizes the useful sequence information while at the same time simplifying the overall data interpretation process. FIG. 26 demonstrates some of the spectral complexity related to the low q CID data of larger RNA molecules (98mer in this example, low q CID of −23 charge state). The advantage here is that complementary 5′ and 3′ fragment data is very useful for data interpretation, while on the other hand the chances for spectral overlap can increase with increasing RNA size sometimes making data interpretation more difficult.

### Example 4

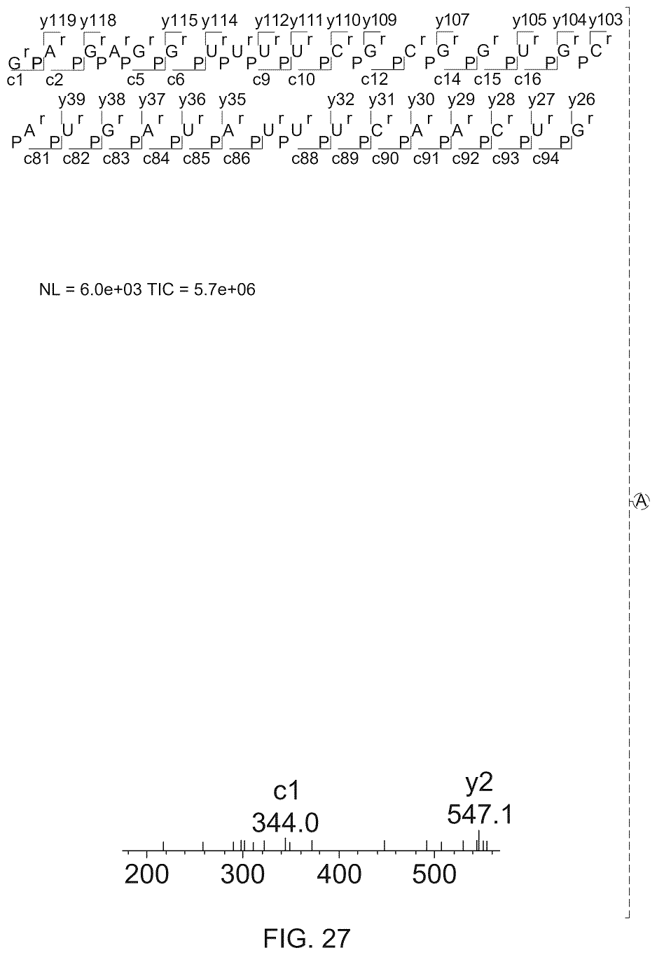

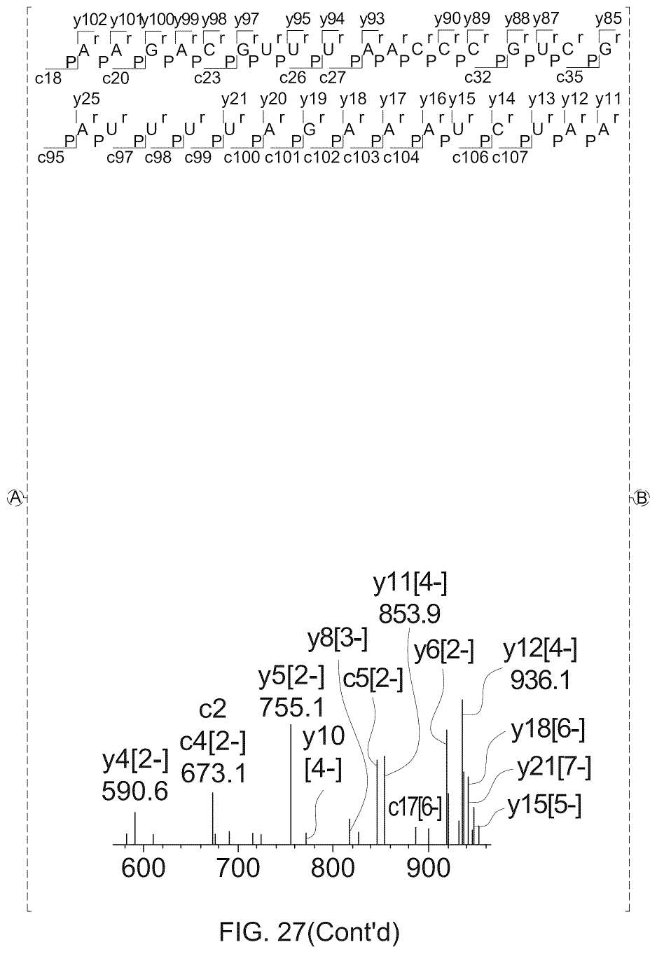

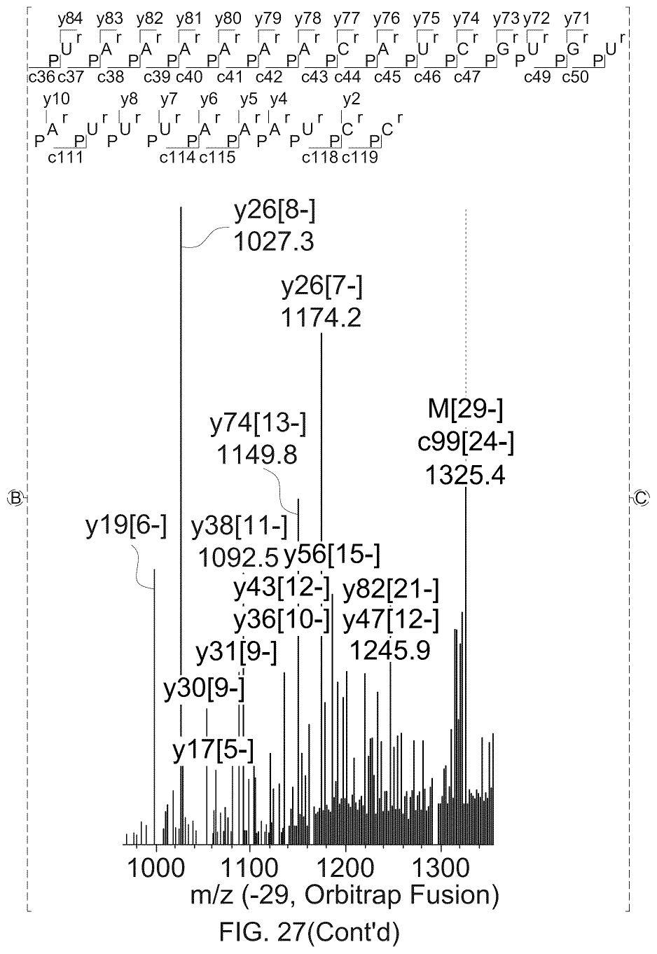

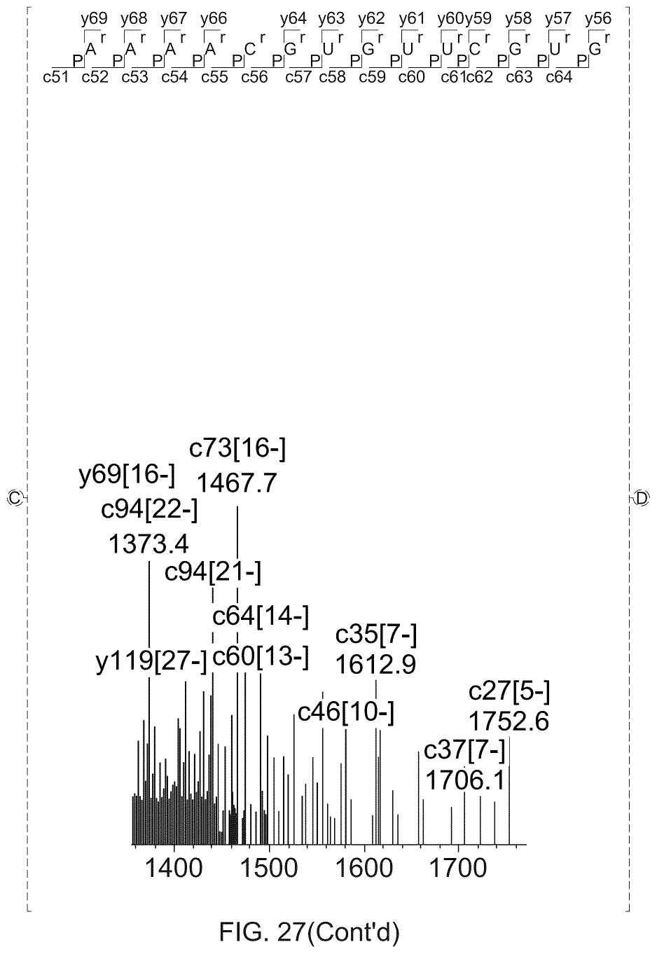

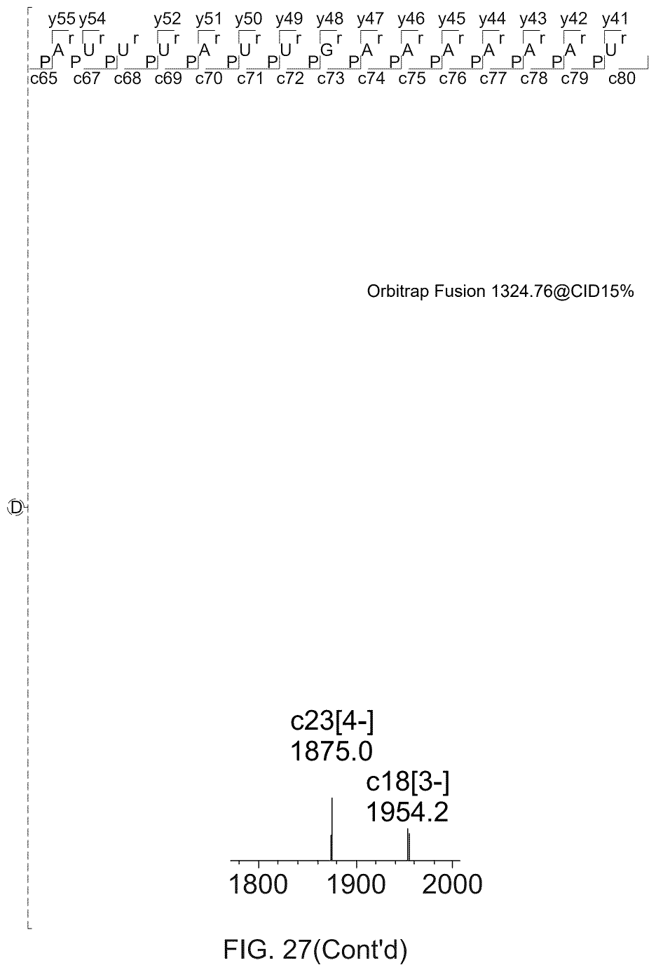

FIG. 27 is the product ion mass spectrum of an RNA 111mer having SEQ ID NO: 17 produced by an aspect of the method disclosed herein comprising low q CID (q=0.15 of the −29 charge state). Similar to FIG. 26, FIG. 27 demonstrates both the advantages and disadvantages of the low q CID approach in terms of sequence coverage originating from both the 5′ and 3′ ends of larger intact RNA sequences and the potential for spectral overlap associated with this type of analysis.

### Example 5

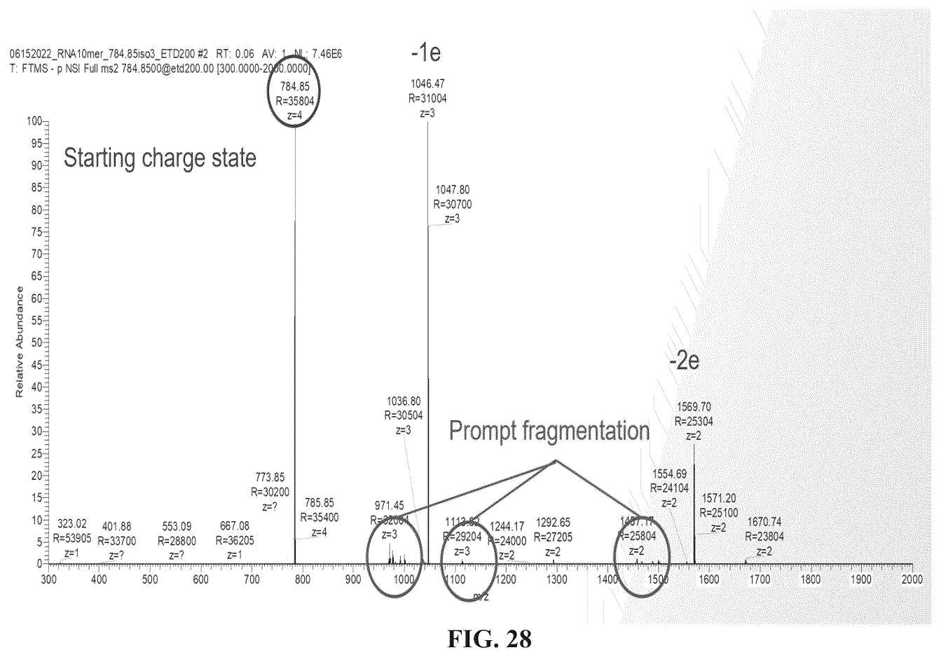

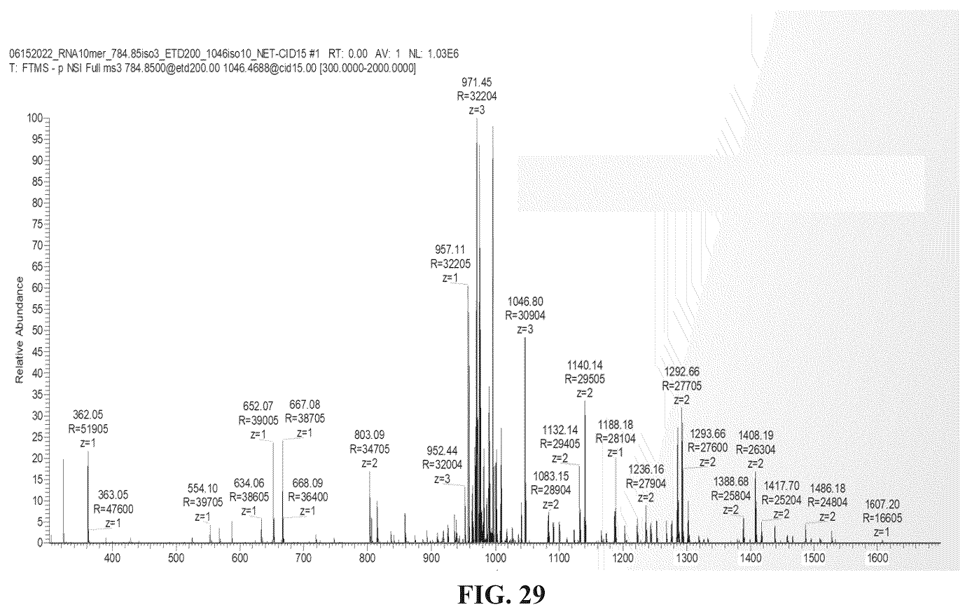

This example concerns negative ion electron transfer dissociation (NETD) of RNA ions. FIG. 28 shows the NETD product ion spectrum of an RNA 10mer having SEQ ID NO: 2 derived from mass isolating the −4 charge state of the 10mer. The −4 charge state of the 10mer was reacted with fluoranthene to produce the data shown in FIG. 28. The major product ions observed where those associated electron transfer and no corresponding dissociation referred to as ETnoD. The peak at m/z 1046.47 represents the transfer of one electron to fluoranthene resulting in the odd electron species at the −3 charge state. The loss of two electrons from consecutive ETnoD reactions can be seen at m/z 1569.70 at the −2 charge state. Prompt fragmentation from the NEDT process can be seen in the product ion spectrum as well albeit at very low levels. The efficient production of this intact odd electron species (representing the transfer of an electron) can also undergo an additional stage of mass spectrometry. By applying the low q CID approach to an odd electron species relevant sequence information derived from different dissociation channels can be obtained. The information obtained is complementary to the low q CID approach described initially in this application. For example in FIG. 29, the odd electron species found at m/z 1046.47 from the initial ETnoD experiment can be mass isolated and fragmented at a q value of 0.15. The resulting product ion mass spectrum is comprised of mainly d/w ions with the addition of a/z fragments as well. This information is complementary to the even electron low q CID approach described earlier and can help confirm RNA sequences directly.

### Example 6

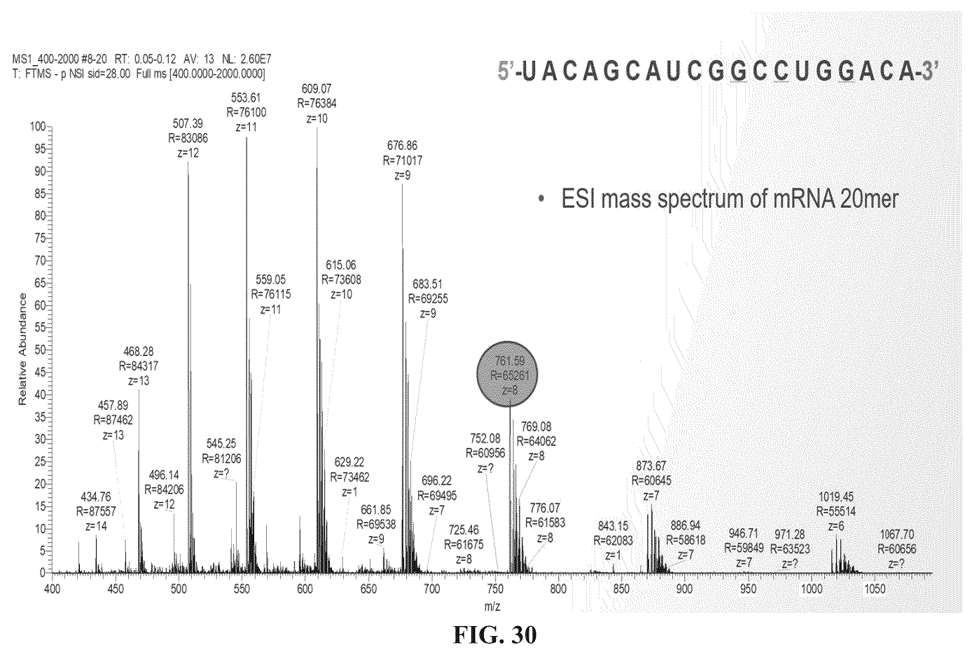

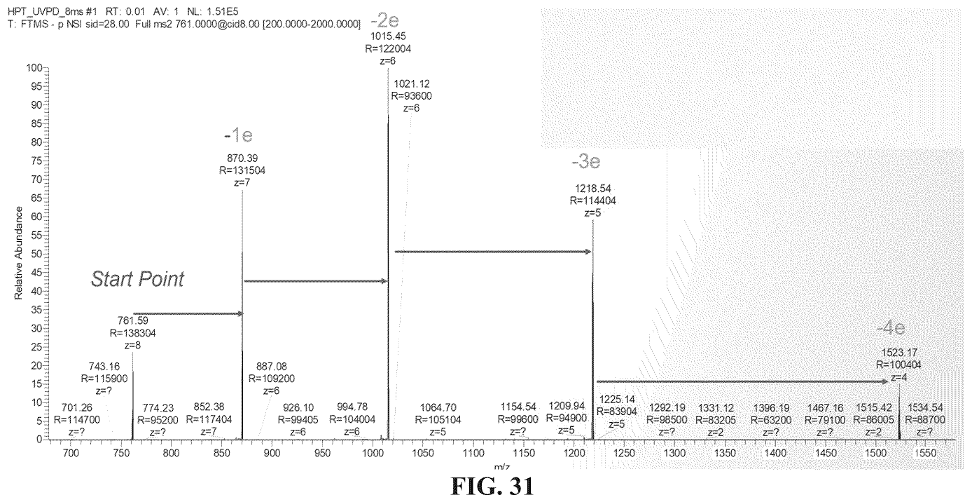

In this example, an ultraviolet (UV) laser was used to detach an electron from an isolated precursor RNA ion for subsequent low q CID. This is a second method presented here to generate odd electron species for low q CID. FIG. 30 shows an ESI mass spectrum for an RNA 20mer (SEQ ID NO: 5) having sequence 5′-U A C A G C A U C G G C C U G G A C A U-3′. FIG. 31 shows the resulting product ion mass spectrum derived from isolating the −8 charge state of the 20mer and exposing it to a 213 nm UV laser for 8 ms. This reaction took place in the high pressure linear ion trap described in FIG. 1. The arrows indicate four successive electron detachment reactions associated with the laser exposure time. In the high pressure linear ion trap, very little prompt fragmentation (ultraviolet photodissociation or UVPD) is observed of the isolated-8 charge state or any of the subsequent four electron detachment product ions. The resulting single electron photodetachment ion at m/z 870.39 can then be subjected to further structural query using low q CID.

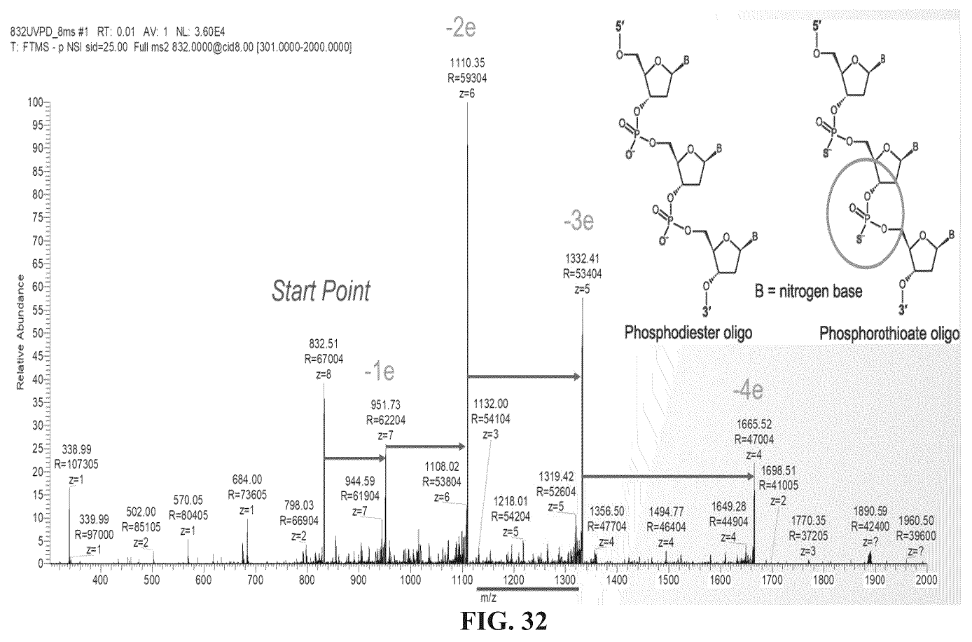

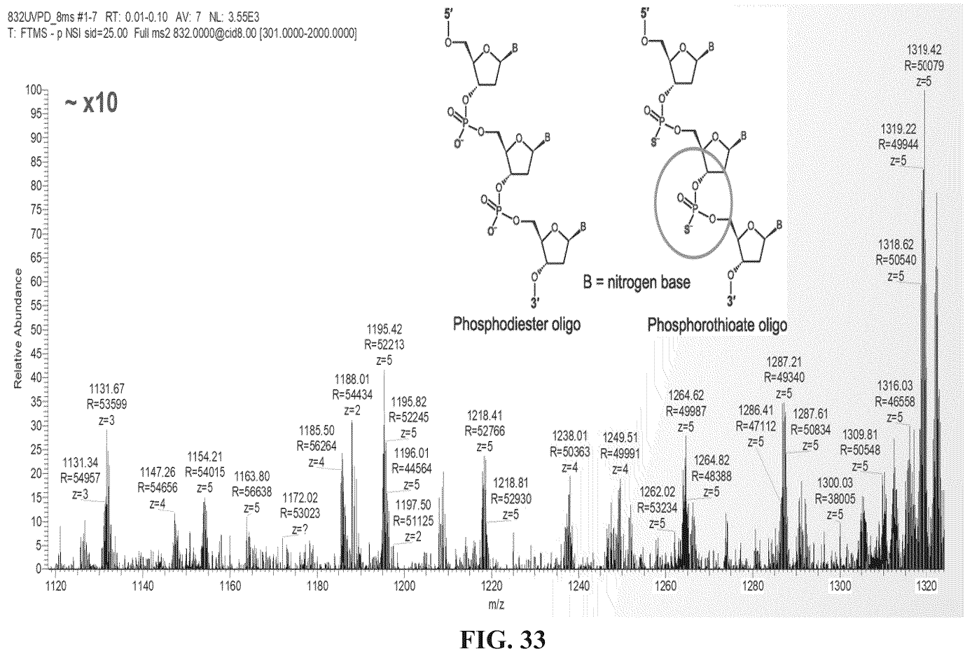

One of the key modifications of therapeutic RNA molecules is the substitution of a sulfur atom into the phosphodiester backbone of RNA producing a highly stable piece of RNA with what is known as a phosphorothioate backbone (see inset of FIG. 32). In FIG. 32 is shown the product ion mass spectrum of a 20mer with a phosphorothioate backbone obtained by isolating the −8 charge state at m/z 832.51 and exposing it to the UV laser at 213 nm for 8 ms. Similar to the previous example a series of four consecutive photodetachment peaks are observed labeled as −1e, −2e, −3e and −4e. In addition substantially more prompt fragmentation of this RNA 20mer is observed due to the higher molar extinction coefficient the phosphorothioate backbone has at 213 nm. The m/z range between 1120 and 1320 is underscored in FIG. 32 with a 10× magnified view shown in FIG. 33 to demonstrate the higher degree of prompt fragmentation observed even in the high pressure trap.

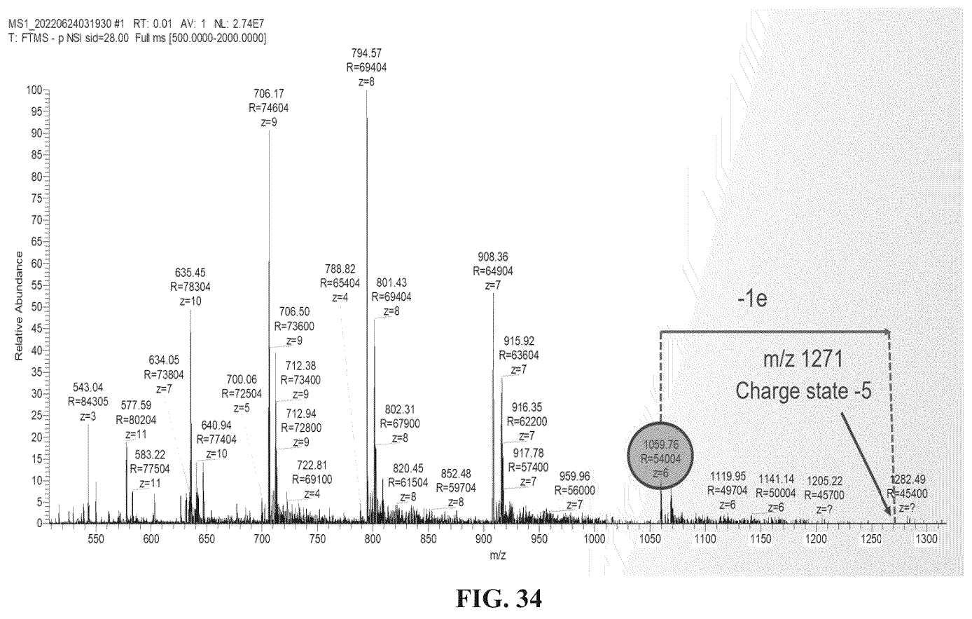

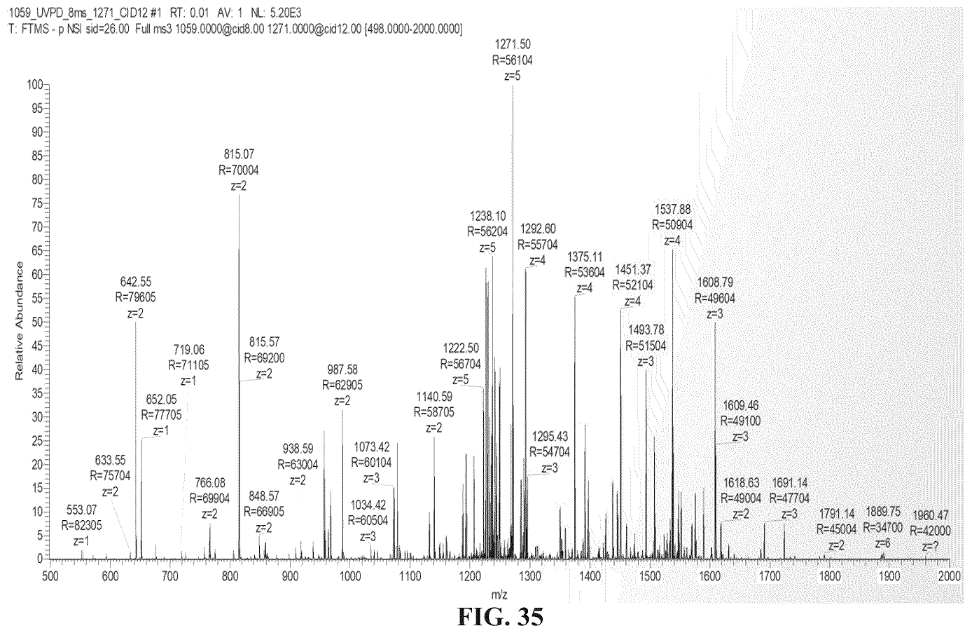

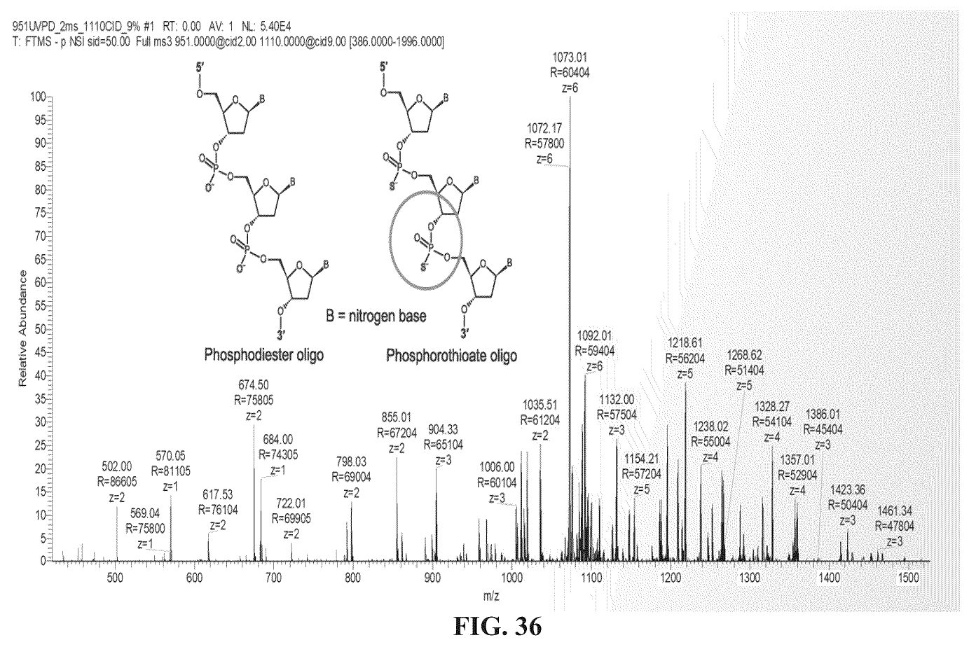

FIG. 34 shows the full scan ESI mass spectrum of an RNA 20mer with a typical phosphodiester backbone. Next the −6 charge state at m/z 1059.76 is mass isolated and exposed to the UV laser for 5 ms, enough time to then generate a substantial-5 charge state odd electron species through the photodetachment process. This peak at m/z 1271.50 is then mass isolated and subjected to low q CID (q=0.15) as shown in FIG. 35. The resulting product ion spectrum is primarily d/w ions with some a/z series as well. FIG. 36 follows the same experimental procedure as that for FIGS. 34 and 35 except that the backbone of the RNA 20mer contains phosphorothioates. The product ion mass spectrum in FIG. 36 is that of the odd electron species generated via photodetachment followed by low q CID of m/z 951.0 to produce d/w and some a/z series ions. In all examples the use of photodetachment combined with low q CID produces complementary fragments to that of the standard low q CID of even electron species.

### Example 7

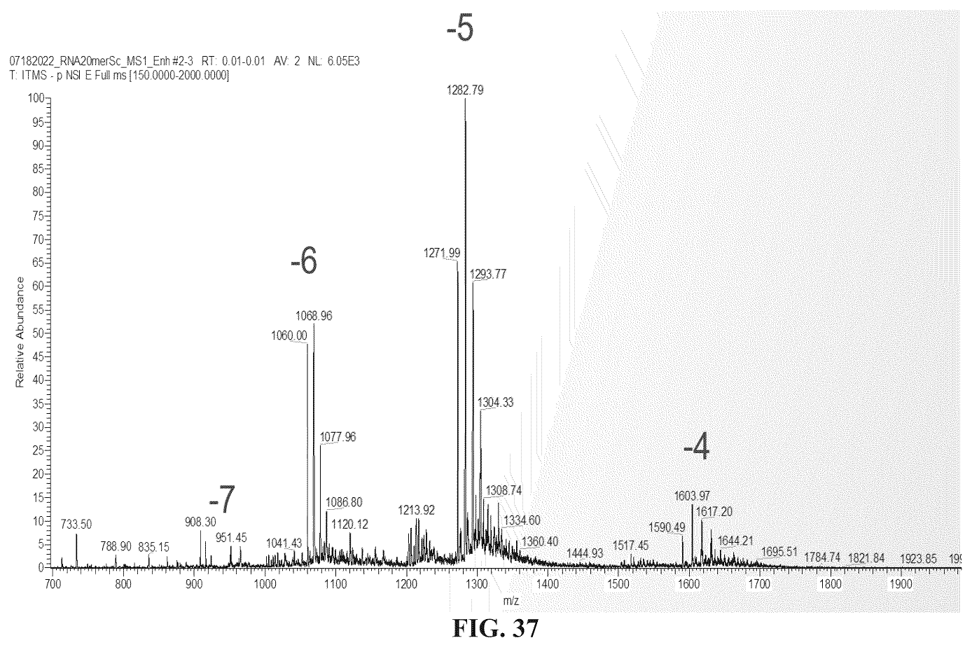

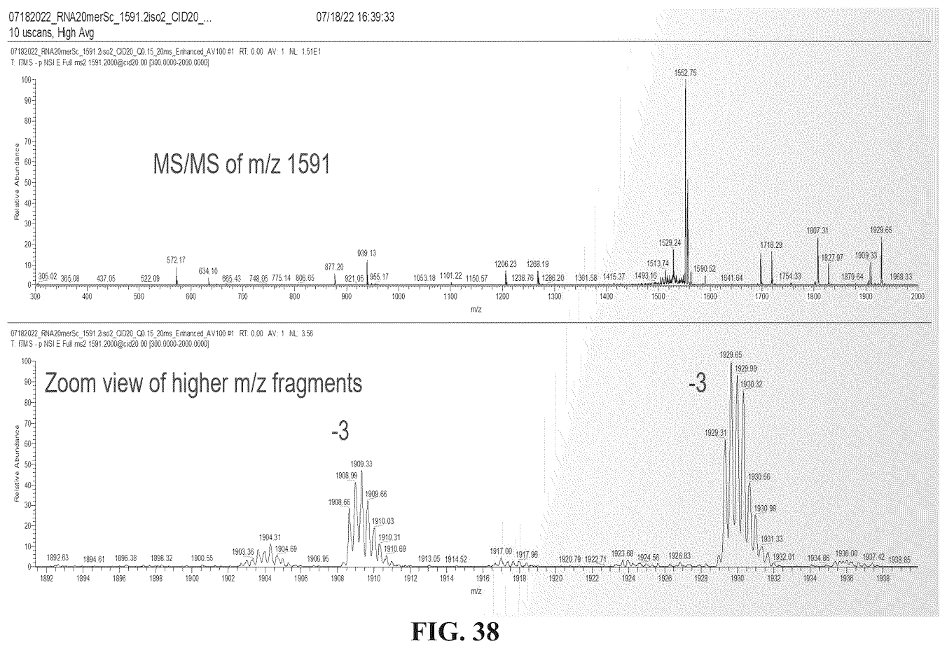

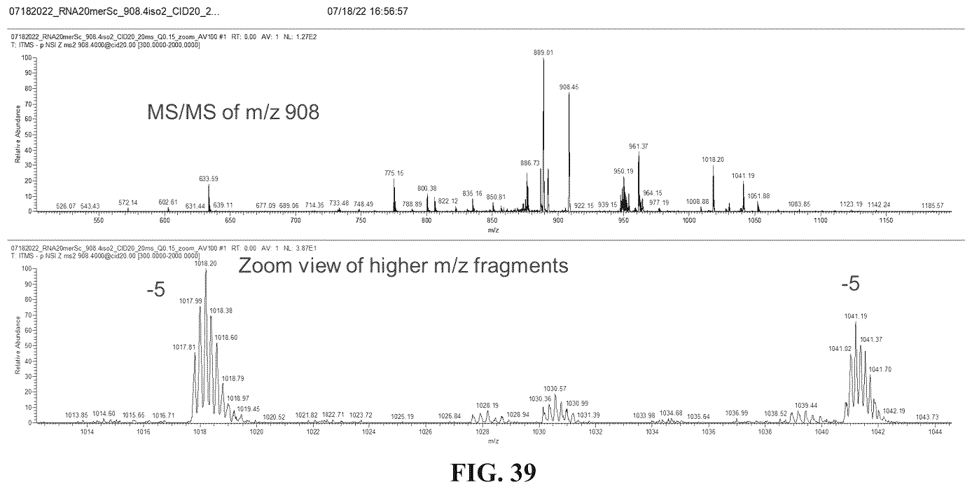

In this set of examples, a dual linear ion trap configuration was used to dissociate and detect an RNA 20mer having SEQ ID NO: 5 to demonstrate the utility of the linear ion trap of as both a dissociation cell and mass analyzer. The dual linear ion trap configuration included a high pressure ion trap for the collision-induced dissociation process and a second trap at lower pressure (slots in the hyperbolic rods) for direct detection of the product ions generated in the high pressure trap. The resolution of the product ion mass spectrum can be controlled by the scan speed of the low pressure trap with slower scanning providing higher resolution data, which is known in the state of the art. FIG. 37 shows the full scan ESI mass spectrum of an RNA 20mer ranging from a charge state of −7 to −4 using direct infusion as a sample introduction method. The low q CID mass spectrum of the −4 charge state of the 20mer at m/z 1590.49 is shown in FIG. 38. The top half of the spectrum demonstrates the sequence specific c/y complementary ion pairs produced hat are typical of the low q CID process. The lower half of FIG. 38 shows for the product ions at m/z 1909 and 1929 typical resolution that can be obtained at normal scan speeds with the instrumental configuration described in FIG. 1. In FIG. 39 is shown the low q CID mass spectrum of the −7 charge state at m/z 908. As with FIG. 38, high quality sequence specific data is obtained with the low q CID experiment. For this example the scan speed was reduced using the enhanced scan function associated with the Tribrid™ mass spectrometry instrument described in FIG. 1. The lower half of the figure shows isotopic resolution of the −5 charge state of two product ions at m/z 1018 and m/z 1042 respectively at the lower scan speed. The use of slower scan speeds can enable the use of the linear ion trap to easily mass analyze RNA 20mers fragmented by low q CID and even somewhat larger species on a routine basis. In addition the use of the conversion dynode/electron multiplier detection scheme can be approximately an order of magnitude greater in sensitivity than electrostatic (e.g., Orbitrap™ analyzer) based detection methods.

### Example 8

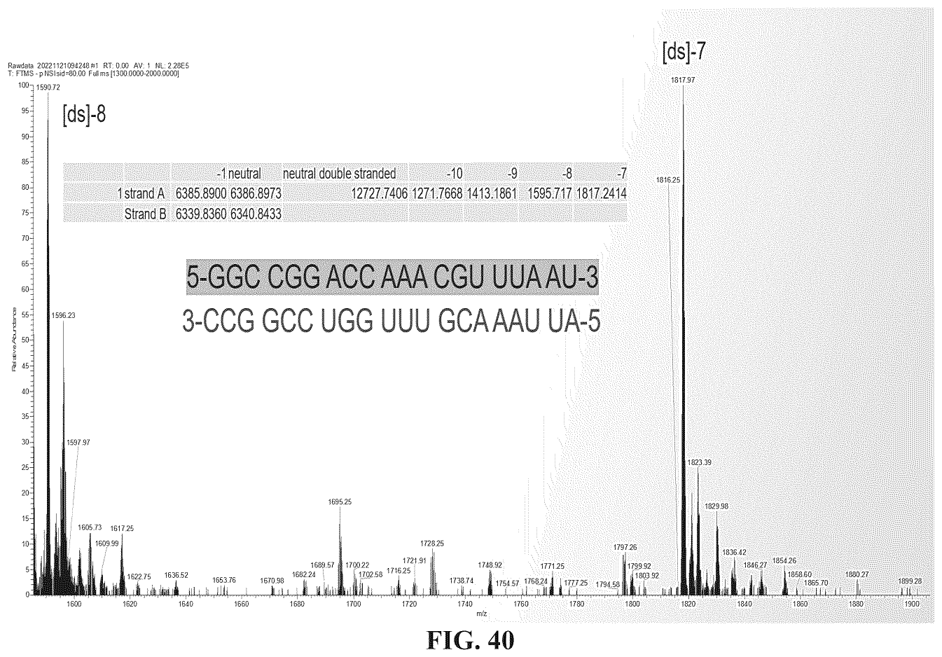

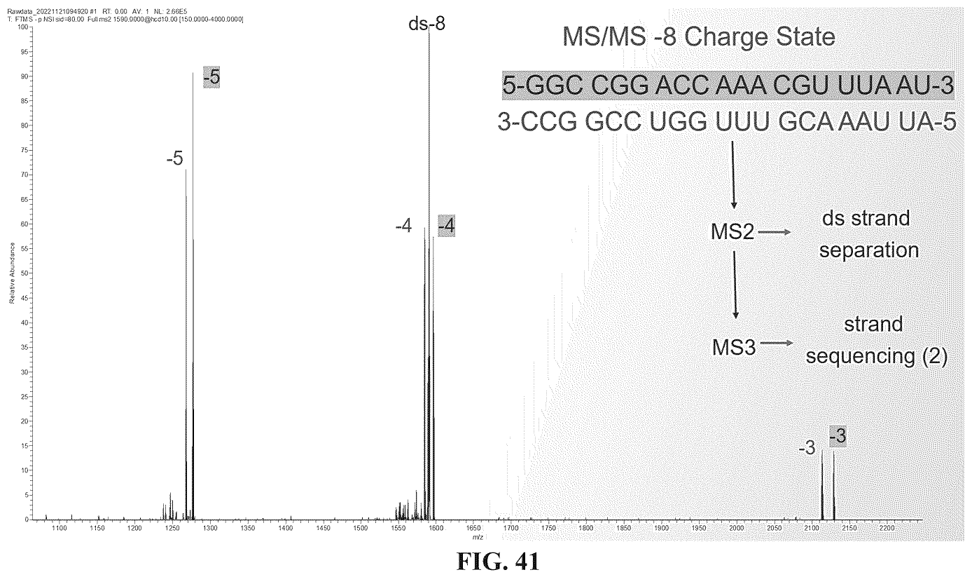

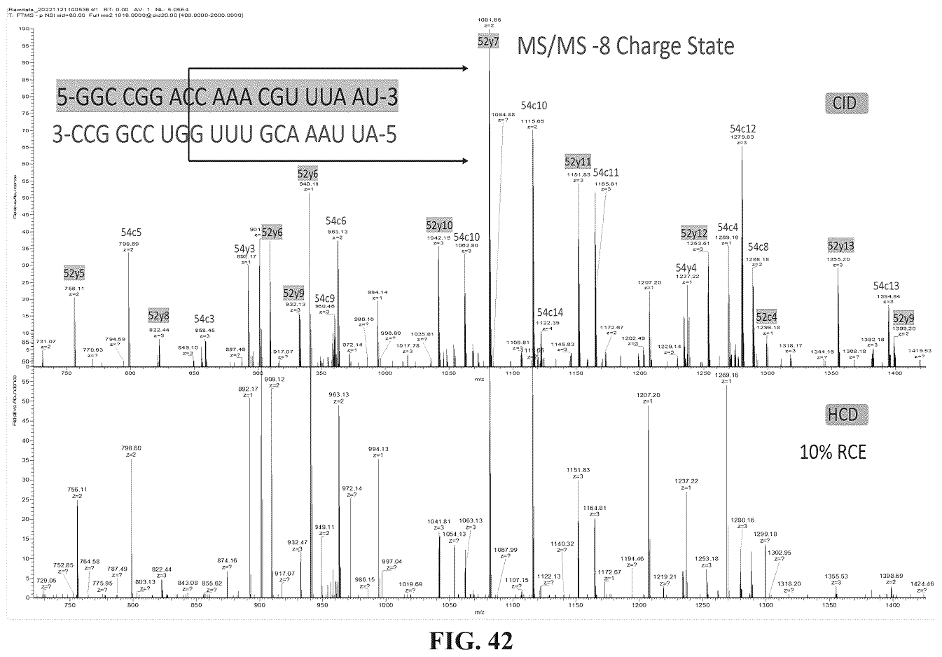

This example concerns analyzing double stranded (ds) RNA and sequencing both strands, which can be used for retrovirus studies, next generation herbicides and pesticides, gene regulation and other applications. The double stranded RNA (DS RNA), where the 5′ to 3′ strand has SEQ ID NO: 12 (5′ G-G-C-C-G-G-A-C-C-A-A-A-C-G-U-U-U-A-A-U 3′) and the 3′ to 5′ strand has sequence C-C-G-G-C-C-U-G-G-U-U-U-G-C-A-A-A-U-U-A 5′. In FIG. 40 is the ESI mass spectrum of dsRNA for this set of examples. Two charge states are observed at −8 and −7 with m/z values of 1590.72 and 1817.97 respectively. Next, the −8 charge state is mass isolated and subjected to standard CID at a q=0.25 using just enough energy to separate the two RNA strands as shown in FIG. 41. Here three pairs of product ions are produced across three different charge states ranging from −5 to −3 with each pair containing the single stranded RNA 3′-5′ and 5′-3′ intact strands. After separating the RNA strands, any strand/charge state can be mass isolated and sequenced using the low q CID experiment. In addition dsRNA can be sequenced directly as illustrated in FIG. 42. Here the dsRNA at the −8 charge state is mass isolated and fragmented using both CID and HCD. Interestingly fragments from the A/U rich reason are readily observed product ions whereas the G/C rich reason exhibited little or no fragmentation under these fixed conditions. This is due most likely to the increased stability of the double helix associated with three hydrogen bond pairs for G/C interactions versus only two for the A/U pairs.

### Example 9

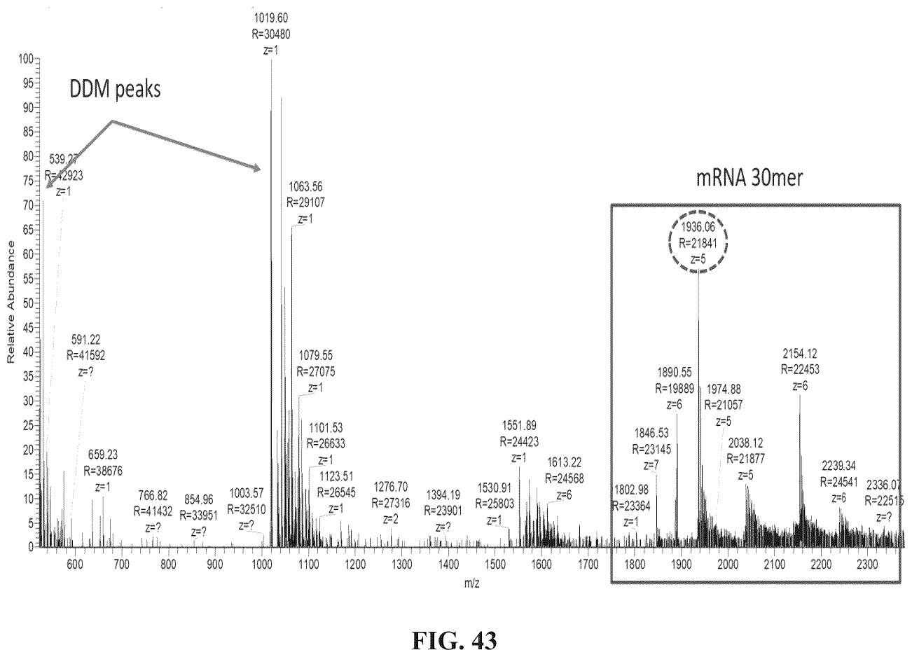

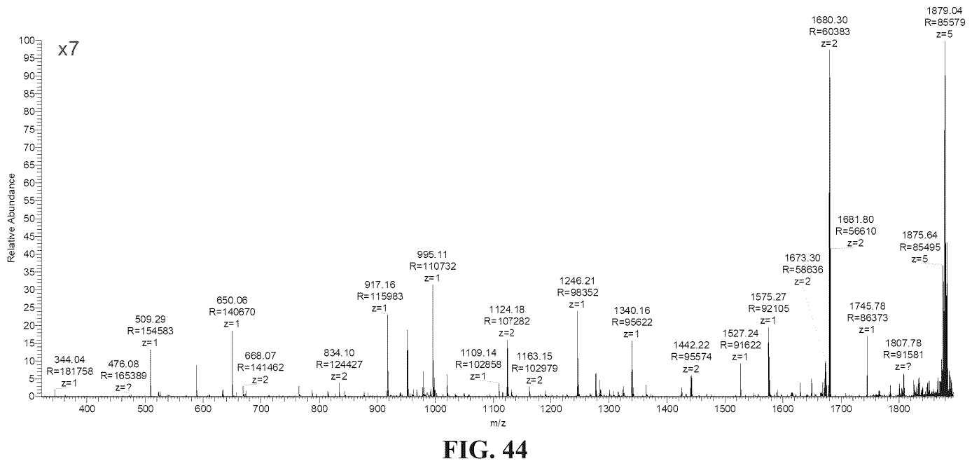

In many instances, RNA molecules are contained by some form of delivery system in order to target the organ of interest for gene therapy, cancer treatments, vaccination purposes, or other medical applications. This example concerns direct sequencing of an mRNA 30mer having SEQ ID NO: 8 in a micelle delivery system using electron spray ionization (ESI), and lipid/micelle removal via collisional heating (CID) in order to analyzer the RNA 30mer directly. An mRNA 30mer (SEQ ID NO: 8): 5′ A-A-U-G-A-U-U-U-A-A-A-U-A-A-U-U-U-A-A-A-A-G-U-U-C-C-U-A-C-U-3′ was added to a mixture comprising dodcyl-β-D-maltoside or DDM (critical micelle concentration of about 0.17 mM) to form a DDM micelle comprising the mRNA 30mer. FIG. 43 shows a mass spectrum of intact micelles introduced into the instrument via ESI following CID of the micelles to yield the monomer/dimer peaks of DDM plus the intact-5 charge state of the RNA 30mer at m/z 1936.06. The −5 charge state can then be mass isolated and subjected to low q CID as illustrated in FIG. 44. The scale is multiplied by a factor of seven illustrating c/y sequence series ions.

### Example 10

For most CID experiments a Mathieu activation q of 0.25 represents a compromise between producing enough collision energy to induce fragmentation while allowing for a sufficiently large enough mass range of product ions to be trapped in the device for subsequent mass analysis. However, for the collision induced dissociation of multiply-charged oligonucleotide anions, the kinetic energy associated a q=0.25 leads to ions with higher velocity and hence larger kinetic energy which can over fragment oligonucleotide anions. By reducing the q value to 0.15 ion velocities are reduced significantly resulting in “fine tuning” the kinetic energy which is converted to vibrational energy in a CID experiment. This fine tuning maximizes the production of complementary c/y ion pairs for the CID of unmodified RNA anions. This same approach can be applied to odd electron species of moderate to heavily modified RNA anions to produce primarily d/w and some a/z ions.

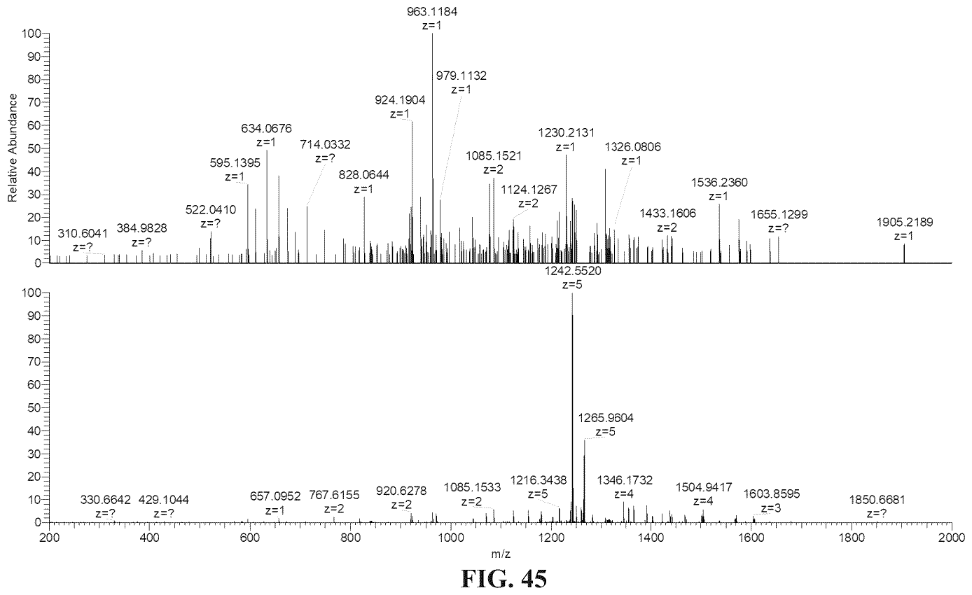

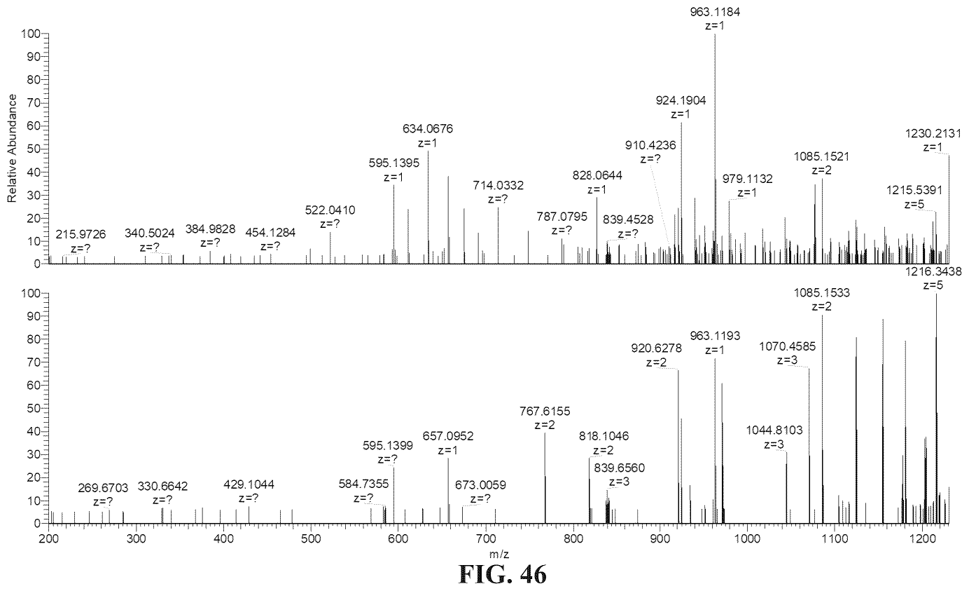

In this example, a sample of an RNA 20mer having SEQ ID NO: 6 was diluted to 1 mM with RNase free water and multiple aliquots were stored at −80° C. The sample was further diluted to 10 μM in 50 mM ammonium acetate for static spray analysis. In FIG. 45 is shown the CID mass spectra for both the q=0.25 and 0.15 respectively (top and bottom) derived from the −5 charge state of an RNA 20mer. The higher ion activation q data is characterized by a wide variety of product ions primarily at much lower charge states than the precursor ion indicating a large degree of sequential fragmentation. This leads to much less specificity for sequencing and can produce a host of internal fragment ions which can complicate peak assignment (top). On the other hand, at a reduced activation q of 0.15, the spectrum is characterized by single cleavage points along the phosphodiester backbone thus producing a series of complementary c/y ion pairs. In addition, the product ions produced are much closer in charge state to that of the precursor ion due to the fine control of the resonance excitation process. The sequence coverage obtained for the low q experiment is much higher than that of the q=0.25, owing to the limited degree of sequential fragment production. FIG. 46 is a zoom view up to ~m/z 1200 or the experiment described in FIG. 45. For the low q CID data (bottom) there are many structurally informative fragments between m/z 1000 and m/z 1200 whereas for a q=0.25 (top), these fragments have been dissociated further due to excess vibrational energy imparted to the precursor ion. FIG. 45 shows the harsh fragmentation mass spectrometry data for a 20mer using HCD conditions (top) versus the soft fragmentation mass spectrometry data for the 20mer in ion trap resonant excitation conditions (bottom). The different spectra demonstrate that the oligonucleotide fragmentation is highly sensitive to fragmentation harshness, the method of activating ions impacts mass spectrometry data, and over-activation generates uninformative ions. With reference to the soft fragmentation mass spectrometry data of FIG. 45, the base loss has a mass to charge ratio of 1242.5520 and the water loss has a mass to charge ratio of 1265.9604. FIG. 46 provides zoomed mass spectrometry data of FIG. 45 from 215 m/z to 1231 m/z further comparing harsh fragmentation to soft fragmentation and the effects of each on producing mass spectral data.

It will be apparent that the precise details of the methods described may be varied or modified without departing from the spirit of the described aspects of the disclosure. We claim all such modifications and variations that fall within the scope and spirit of the claims below.

### V. Enumerated Paragraphs

Examples of inventive subject matter according to the present disclosure are described in the following enumerated paragraphs.
A1. A method for performing ion trap type resonant collision induced dissociation (CID) on labile compounds to drive selective dissociation along desired dissociation pathways, the method comprising:
   injecting from an external source, creating within or otherwise providing a population of ions into a confinement volume of a dissociation cell, wherein:
      said population of ions comprises ions of a labile compound with multiple dissociation pathways that produce different types of bond cleavages and different types of product ions; and
      wherein the dissociation cell provides a substantially quadrupolar radio frequency (RF) electrostatic confinement potential in said confinement volume of the dissociation cell during kinetic activation of selected precursor ions in the population of ions;
   kinetically activating the precursor ions in one or more of the dimensions of the substantially quadrupolar confinement potential such that the precursor ions undergo collisions with a buffer gas present in the confinement volume of the dissociation cell, wherein the collisions cause:
      the precursor ions to selectively dissociate without being ejected from the confinement region; and
      at least some resulting product ions from the selective dissociation to remain confined in the dissociation cell.
A1.1. The method of paragraph A1, wherein said population of ions is, upon collisional activation, likely to yield product ion types that result from multiple bond cleavages of the labile compound ions.
A1.1.1. The method of paragraph A1.1, wherein the different types of product ions include internal fragment ions, ions that result from dissociation of more than one bond of precursor ions, base losses from sequence ions, or a combination thereof.
A1.1.1.1. The method of paragraph A1.1.1, wherein the different types of product ions include other product ions disclosed in the present disclosure.
A1.1.2. The method of any of paragraphs A1.1-A1.1.1.1, wherein the population of ions comprises oligonucleotide ions, and the product ions are predominantly composed of first-generation products.
A.1.1.2.1 The method of A1.1.2, wherein the first-generation products include sequence ions and base losses from the precursor ions in the subset of ions.
A1.2. The method of any of paragraphs A1-A1.1.2, wherein it is desired to promote the production of certain types of product ions and/or suppress the production of said production types from multiple bond cleavages of the precursor labile compound ions.
A2. The method of any of paragraphs A1-A1.2, wherein said RF confinement potential is defined by one or more parameters including q, the Mathieu activation value, a parameter VRE, which is proportional to magnitudes of sinusoidal voltages applied to electrodes of said dissociation cell to establish said RF confinement potential, a parameter ORF, which is an angular frequency of the said RF confinement potential, or at least one characteristic dimension parameter, r₀, that defines how a physical electrode structure and geometry of said dissociation cell scales a magnitude of said RF confinement potential for a given VRE.
A2.1. The method of paragraph A2, wherein in at least one dimension, the RF confinement potential acts as a substantially quadrupolar field.
A2.2. The method of any of paragraphs A2-A2.1, wherein in the at least one dimension, the RF confinement potential provides an electric field, such that an electric field component acting in that dimension varies substantially in proportion to displacement in that dimension with a small percentage of deviation from linearity over a range of displacements that ions can have upon kinetic activation within which the ions are neither ejected from the dissociation cell nor impinge on the field defining electrodes.
A2.3. The method of any of paragraphs A2-A2.2, wherein the parameters V_{RF} and ORF are chosen according to the associated characteristic dimension of the dissociation cell, r₀, such that kinetically activating within the confinement volume where the electric field component varies in proportion to displacement in that dimension for purposes of effecting dissociation of the precursor ions, the precursor ions achieve no more than a maximum magnitude of velocity |v|ₘₐₓ, does not impinge on an electrode, and is not ejected from the device.
A2.3.1. The method of paragraph A2.3, wherein the value of |v|ₘₐₓ depends upon a mass of a buffer gas in the confinement volume.
A2.3.2. The method of any of paragraphs A2.3-A2.3.1, wherein the value of |v|ₘₐₓ includes velocity along multiple dimensions in which the ions are being kinetically activated simultaneously.
A2.3.4. The method of any of paragraphs A2.3-A2.3.2, wherein the value of |v|ₘₐₓ depends upon the type of ionized molecules being analyzed.
A2.3.5. The method of any of paragraphs A2.3-A2.3.4, wherein |v|ₘₐₓ is determined as

$${\vert 𝓋 \vert}_{max} = \frac{\sqrt{3} {q}_{act} {ω}_{R F} {x}_{max}}{\sqrt{2 ( 1 + 0.90804633 {q}_{act} )}}$$

A.2.3.6 The method of any of paragraphs A2.3-A2.3.4, wherein |v|ₘₐₓ for a population of labile precursor ions including deprotonated RNA ions when using helium as the buffer gas is within a range of 2870 to 3640 m/sec, or more preferably about 3070 m/sec.
A.2.3.7 The method of any of paragraphs A2.3-A2.3.4, wherein ∥max for a population of labile precursor ions including deprotonated RNA ions when using diatomic hydrogen as the buffer gas is within a range of 2870 to 3640 m/sec, or more preferably about 3760 m/sec.
A.2.3.8 The method of any of paragraphs A2.3-A2.3.4, wherein |v|ₘₐₓ for a population of labile precursor ions including deprotonated RNA ions when using diatomic nitrogen as the buffer gas is within a range of 1480 to 1890 m/sec, or more preferably about 1680 m/sec.
A.2.3.9 The method of any of paragraphs A2.3-A2.3.4, wherein |v|ₘₐₓ for a population of labile precursor ions including deprotonated RNA ions when using argon as the buffer gas is within a range of within a range of 1270 to 1680 m/sec, or more preferably about 1460 m/sec.
A3. The method of any of paragraphs A1-A2.3.9, wherein selectively dissociating corresponds to the precursor ions fragmenting into initial product ions substantially without the product ions undergoing further fragmentation.
A4. The method of any of paragraphs A1-A3, wherein selectively dissociating corresponds to causing the precursor ions to achieve resonant activation, and wherein the fragmentation of the precursor ions into the product ions causes the product ions to fall out of resonance. A5. The method of any of paragraphs A1-A4, wherein selectively dissociating corresponds to the precursor ions preferentially fragmenting through desired dissociation pathways.
A6. The method of any of paragraphs A1-A5, wherein the selective dissociation is controlled by a parameter based in part on one or more of a mass of a buffer gas in the dissociation cell, a mass-to-charge ratio of the precursor ion, a radius of the confinement volume, a resonance RF frequency of the precursor ion, and/or a Mathieu q value for the dissociation cell.
A6.1. The method of paragraph A6, wherein the selective dissociation parameter is determined for ions of the labile compound based on a combination of one or more of the m/z of the precursor ions, the precursor ion resonant frequency, the radius of the confinement volume of the dissociation cell, dimensions of the dissociation cell, characteristics of a buffer gas in the confinement volume, and a trap operating frequency.
A6.2. The method of any of paragraphs A1-A6.1, wherein the selective dissociation parameter is selected to cause the precursor ions to undergo only a lowest energy backbone fragmentation.
A6.3. The method of any of paragraphs A6-A6.2, wherein the selective dissociation parameter is further determined for the labile compound based on its compound class.
A6.4. The method of any of paragraphs A6-A6.3, wherein the selective dissociation parameter is defined by a product of the radius and the resonant RF frequency for the precursor ions.
A6.4.1. The method of paragraph A6.4, wherein the selective dissociation parameter is further defined by the product as scaled by the mass of the buffer gas in the dissociation cell.
A6.4.2. The method of any of paragraphs A6.4-A6.4.2, wherein the selective dissociation parameter for the at least one labile compound corresponds to a kinetic energy described by:

$${{m}_{g a s} [ {r}_{0} \frac{{ω}_{R F}}{2} \frac{{q}_{act}}{\sqrt{2}} ]}^{2} .$$

A6.5. The method of any of paragraphs A6-A6.4, wherein the selective dissociation parameter is configured such that the precursor ions are driven near but not at an associated resonance frequency.
A6.6. The method of any of paragraphs A6-A6.5, wherein the method further comprises determining the selective dissociation parameter.
A6.6.1. The method of paragraph A6.6, wherein the method further comprises the steps of:
   receiving characteristic information about the at least one labile compound; and
   determining the selective dissociation parameter based in part on the characteristic information (e.g., charge state, chemical property, mass to charge, compound type).
A7. The method of any of paragraphs A1-A6.5, wherein the Mathieu activation q for the dissociation cell is at or below 0.22, 0.2, 0.18, or 0.15.
A7.1. The method of paragraph A7, wherein the Mathieu activation q for the dissociation cell is determined by the steps of:
   determining a highest Mathieu activation q value where the precursor ions are ejected from the confinement volume rather than, or before, yielding undesirable dissociation or fragmentation pathways; and
   setting the Mathieu activation q for the dissociation cell to a value between 60%-95% of the highest Mathieu activation q value.
A8. The method of any of paragraphs A1-A7.1, further comprising selecting a maximum auxiliary AC potential frequency to be applied to the dissociation cell to cause the precursor ions to selectively dissociate based upon a Mathieu q value.
A9. The method of any of paragraphs A1-A8, wherein the precursor ions comprise a macromolecule.
A9.1. The method of paragraph A9, wherein the precursor ions comprise a polymer or biopolymer.
A9.2. The method of any of paragraphs A1-A9.1, wherein the precursor ions comprise an oligonucleotide.
A9.2.1. The method of paragraph A9.2, wherein precursor ions are oligonucleotide anions ionized through deprotonation.
A9.2.2. The method of any of paragraphs A9.2-A9.2.1, wherein the oligonucleotide anions ionized through deprotonation have been rendered electron deficient.
A9.2.2.1. The method of paragraph A9.2.2, wherein the electron deficient deprotonated oligonucleotide anions were produced through photo-electron detachment from oligonucleotide anions ionized through deprotonation.
A9.2.2.2. The method of any of paragraphs A9.2-A9.2.2.1, wherein the electron deficient deprotonated oligonucleotide anions were produced through electron transfer (abstraction) ion-ion reactions of oligonucleotide anions ionized through deprotonation with suitable negative electron transfer reagent cations.
A9.2.3. The method of any of paragraphs A9.2.1-A9.2.2.2, wherein the oligonucleotide ions are anions.
A9.2.4. The method of any of paragraphs A9.2.1-A9.2.2.3, wherein the precursor ions are one or more of RNA, modified RNA, DNA, Single stranded DNA, synthetic RNA analogs, or a combination thereof.
A9.2.4.1. The method of paragraph A9.2.4, wherein the RNA comprises one or more from the list of mRNA, tRNA rRNA, siRNA, circular RNA, double stranded RNA, RNA-DNA complexes, RNA aptamers, RNA modified with carbohydrates, and RNA-protein complexes.
A9.2.4.2. The method of any of paragraphs A9.2.4-A9.2.4.1, wherein the RNA prior to ionization comprises one or more from the list of saRNA, ribozymes, miRNA, sgRNA, RNA generated via ribonuclease digestion in solution or attached/tethered to beads, and RNA that has a modified 5′ or 3′ terminus, ribose, phosphodiester backbone, or novel base incorporation.
A9.2.5. The method of any of paragraphs A9.2-A9.2.4.1, wherein the minimum ion charge density for the oligonucleotide or precursor ions is less than one of 3.0 bases (mers)/de-protonation, 2.5 bases (mers)/de-protonation, 2.5 bases (mers)/de-protonation, and 2.0 bases (mers)/de-protonation.
A9.3. The method of any of paragraphs A9-A9.2.5, wherein the precursor ions comprise one of DNA, RNA, protein, and/or portions thereof.
A9.4. The method of any of paragraphs A9-A9.3, wherein the precursor ions comprise one of the compounds discussed within the present disclosure.
A10. The method of any of paragraphs A1-A9.4, wherein the dissociation cell generates the substantially quadrupolar field in at least one dimension to perform resonance activated fragmentation on the ions in multiple stages (MS″).
A10.1. The method of paragraph A10, wherein the substantially quadrupolar field is the dominant field effect generated by the dissociation cell.
A10.2. The method of any of paragraphs A10-A10.1, wherein the substantially quadrupole field is the dominant confinement field within the confinement volume.
A10.3. The method of any of paragraphs A10-A10.2, wherein the dissociation cell is a 2D ion trap.
A10.3.1. The method of paragraph A10.3, wherein the dissociation cell is a linear quadrupole trap.
A10.3.2. The method of paragraph A10.3, wherein the dissociation cell is a toroidal ion trap.
A10.3.3. The method of paragraph A10.3, wherein the dissociation cell has a curved axis.
A10.3.4. The method of paragraph A10.3, wherein the dissociation cell is a rf Paul trap.
A10.4. The method of any of paragraphs A10-A10.2, wherein the dissociation cell is a 3D ion trap.
A11. The method of any of paragraphs A1-A10.4, wherein the buffer gas is selected from a list of helium, hydrogen, deuterium, nitrogen and argon.
B1. A computer readable medium storing non-transitory computer readable instructions that, when executed by a processor cause the processor to initiate performance of the method of any of paragraphs A1-A11.
C1. A mass spectrometer system for complex mass spectrometry experiments and measurements using ion trap type resonant collision induced dissociation (CID) on labile compounds to drive selective dissociation along desired dissociation pathways, the system comprising; an ion source; a dissociation cell, a detector, and a memory storing non-transitory computer readable instructions that, when executed on the one or more processors cause the processors to perform the methods of any of paragraphs A1-A11.
D1. Use of the mass spectrometer system of C1 to perform the method of any of the paragraphs A1-A11.

We claim:

1. A mass spectrometer system for complex mass spectrometry experiments and measurements using ion trap type resonant collision induced dissociation (CID), the system comprising:
an ion source;
a dissociation cell;
a detector; and
a memory storing non-transitory computer readable instructions that, when executed on the one or more processors cause the processors to cause the mass spectrometer system to perform a method for ion trap type resonant CID on labile compounds to drive selective dissociation along desired dissociation pathways, the method comprising:
injecting, from the ion source, a population of ions into a confinement volume of the dissociation cell, wherein (i) the population of ions comprises ions of a labile compound with multiple dissociation pathways that produce different types of bond cleavages and different types of product ions, and (ii) the dissociation cell provides a substantially quadrupolar radio frequency (RF) electrostatic confinement potential in the confinement volume of the dissociation cell during kinetic activation of selected precursor ions in the population of ions; and
kinetically activating the precursor ions in one or more dimensions of the substantially quadrupolar confinement potential such that the precursor ions undergo collisions with a buffer gas present in the confinement volume of the dissociation cell, wherein the collisions cause (i) the precursor ions to selectively dissociate without being ejected from the confinement region, and (ii) at least some resulting product ions from the selective dissociation to remain confined in the dissociation cell.

2. The system of claim 1, wherein parameters of the substantially quadrupolar RF confinement potential are chosen such that the precursor ions within the substantially quadrupolar RF confinement potential achieve a velocity no more than a maximum magnitude of velocity |v|ₘₐₓ.

3. The system of claim 2, wherein the parameters include at least one of:
V_{RF}, a voltage proportional to magnitudes of sinusoidal voltages applied to electrodes of the dissociation cell to establish the RF confinement potential,
ω_{RF}, an angular frequency of the RF confinement potential,
q, the Mathieu activation value, and
at least one characteristic dimension parameter, r₀, that defines how a physical electrode structure and geometry of the dissociation cell scales a magnitude of the RF confinement potential for a given magnitude of the applied RF confinement potential establishing voltages applied to electrodes of the dissociation cell.

4. The system of claim 2, wherein |v|ₘₐₓ is determined as
$${\vert 𝓋 \vert}_{max} = \frac{\sqrt{3} {q}_{a c t} {ω}_{R F} {x}_{max}}{\sqrt{2 ( 1 + 0.90804633 {q}_{a c t} )}} .$$

5. The system of claim 2, wherein the value of |v|ₘₐₓ depends upon a mass of a buffer gas in the confinement volume.

6. The system of claim 2, wherein the value of |v|ₘₐₓ includes velocity along multiple dimensions in which the ions are being kinetically activated simultaneously.

7. The system of claim 2, wherein the value of |v|ₘₐₓ depends upon the type of ionized molecules being analyzed.

8. The system of claim 2, wherein |v|ₘₐₓ for a population of labile precursor ions including deprotonated RNA ions when using helium as the buffer gas is within a range of 2870 to 3640 m/sec, or more preferably about 3070 m/sec.

9. The system of claim 1, wherein selectively dissociating corresponds to the precursor ions fragmenting into initial product ions substantially without the product ions undergoing further fragmentation.

10. The system of claim 1, wherein selectively dissociating corresponds to causing the precursor ions to achieve resonant activation, and wherein the fragmentation of the precursor ions into the product ions causes the product ions to fall out of resonance.

11. The system of claim 1, wherein selective dissociation of precursor ions is controlled by a selective dissociation parameter based in part on one or more of:
a mass of a buffer gas in the dissociation cell,
a mass to charge ratio of the precursor ions,
a radius of the confinement volume,
a resonance RF frequency of the precursor ions,
dimensions of the dissociation cell, or
the Mathieu q for the dissociation cell.

12. The system of claim 11, wherein the selective dissociation parameter is defined by a product of the radius and the resonant RF frequency for the precursor ion.

13. The system of claim 11, wherein the selective dissociation parameter for the at least one labile compound corresponds to a kinetic energy defined by:
$${{m}_{g a s} [ {r}_{0} \frac{{ω}_{R F}}{2} \frac{{q}_{a c t}}{\sqrt{2}} ]}^{2} .$$

14. The system of claim 11, wherein the method further comprises determining the selective dissociation parameter, the determining comprising the steps of:
receiving characteristic information about the precursor ion; and
determining the selective dissociation parameter based at least in part on the characteristic information.

15. The system of claim 1, wherein the precursor ion comprises an oligonucleotide, a peptide anion, or a protein anion.

16. The system of claim 1, wherein a Mathieu activation q for the dissociation cell is at or below 0.22, 0.2, 0.18, or 0.15.

17. The system of claim 1, wherein a Mathieu activation q for the dissociation cell is determined by the steps of:
determining a highest Mathieu activation q value where the precursor ions are ejected from the confinement volume rather than, or before, yielding undesirable dissociation or fragmentation pathways; and
setting the Mathieu activation q value for the dissociation cell to a value between 60%-95% of the highest Mathieu activation q value.

18. The system of claim 1, wherein the dissociation cell is a 2D ion trap.

19. A method for performing ion trap type resonant collision induced dissociation (CID) on labile compounds to drive selective dissociation along desired dissociation pathways, the method comprising:
providing a population of ions into a confinement volume of a dissociation cell, wherein (i) the population of ions comprises ions of a labile compound with multiple dissociation pathways that produce different types of bond cleavages and different types of product ions, and (ii) the dissociation cell provides a substantially quadrupolar radio frequency (RF) electrostatic confinement potential in the confinement volume of the dissociation cell during kinetic activation of selected precursor ions in the population of ions; and
kinetically activating the precursor ions in one or more dimensions of the substantially quadrupolar confinement potential such that the precursor ions undergo collisions with a buffer gas present in the confinement volume of the dissociation cell, wherein the collisions cause (i) the precursor ions to selectively dissociate without being ejected from the confinement region; and (ii) at least some resulting product ions from the selective dissociation to remain confined in the dissociation cell.

20. A computer readable medium storing non-transitory computer readable instructions that, when executed by a processor cause the processor to initiate performance of a method for ion trap type resonant CID on labile compounds to drive selective dissociation along desired dissociation pathways, the method comprising:
providing a population of ions into a confinement volume of a dissociation cell, wherein (i) the population of ions comprises ions of a labile compound with multiple dissociation pathways that produce different types of bond cleavages and different types of product ions, and (ii) the dissociation cell provides a substantially quadrupolar radio frequency (RF) electrostatic confinement potential in the confinement volume of the dissociation cell during kinetic activation of selected precursor ions in the population of ions;
kinetically activating the precursor ions in one or more dimensions of the substantially quadrupolar confinement potential such that the precursor ions undergo collisions with a buffer gas present in the confinement volume of the dissociation cell, wherein the collisions cause (i) the precursor ions to selectively dissociate without being ejected from the confinement region, and (ii) at least some resulting product ions from the selective dissociation to remain confined in the dissociation cell.
